(19) United States
(12) Patent Application Publication
(10) Pub. No.: US 2024/0116963 A1
(43) Pub. Date: Apr. 11, 2024

(54) DENDRIMERS HAVING THIOPHOSPHORAMIDATE PATTERNS AND DERIVATIVES

(71) Applicants: CENTRE NATIONAL DE LA RECHERCHE SCIENTIFIQUE - CNRS -, Paris (FR); UNIVERSITE PAUL SABATIER TOULOUSE III, Toulouse (FR); INSERM (INSTITUT NATIONAL DE LA SANTÉ ET DE LA RECHERCHE MÉDICALE), Paris (FR)

(72) Inventors: Abdelouahd OUKHRIB, Toulouse Cedex (FR); Julien AUJARD-CATOT, Toulouse Cedex (FR); Rèmy POUPOT, Toulouse Cedex 3 (FR); Cèdric-Olivier TURRIN, Toulouse Cedex (FR)

(21) Appl. No.: 17/788,114

(22) PCT Filed: Dec. 22, 2020
(86) PCT No.: PCT/FR2020/052609

§ 371 Date: Jun. 22, 2022

(30) Foreign Application Priority Data
Dec. 23, 2019 (FR) FR19 15518

(51) Int. Cl. C07F 9/6593; A61P 29/00

(52) U.S. Cl. CPC C07F 9/65815; A61P 29/00

(57) ABSTRACT

The present invention relates to new dendrimers with thiophosphoramidate units and derivatives, their preparation method and their use.

### TECHNICAL FIELD

The present invention relates to new dendrimers with thiophosphoramidate units and derivatives, their preparation method and their use.

### PRIOR ART

Dendrimers are macromolecules consisting of monomers which join together by a branching process around a plurifunctional central core.

Dendrimers, also referred to as “cascade molecules” or “arborols”, are highly branched functional polymers with a defined structure. These macromolecules are effectively polymers because they are based on the association of repeat units. However, dendrimers differ fundamentally from conventional polymers in so far as they have properties due to their branching construction. The molecular weight and the shape of dendrimers can be precisely controlled, and all the functions are located at the end of the branches, forming a surface, which makes them readily accessible.

Dendrimers are constructed step-by-step, by repeating a sequence of reactions enabling the multiplication of each repeat unit and the terminal functions. Each reaction sequence forms what is referred to as a “new generation”. The branching construction is produced by repeating a sequence of reactions which makes it possible to obtain, at the end of each reaction cycle, a new generation and an increasing number of identical branches. After a few generations, the dendrimer generally assumes a globular, highly branched form which is plurifunctionalized by virtue of the numerous terminal functions present at the periphery.

These structural characteristics have thus reinforced their use in biology and medicine (Cloninger M. J. Curr. Opin. Chem. Biol. 2002, 6, 742-748). Dendrimers can be used as medicament vectors, capable of improving the solubility thereof in water, improving transport of the medicament across biological barriers, and ensuring the distribution of the medicament in tissues (Lee C. C. et al. Nat. Biotechnol. 2005, 23, 1517-1526; Cheng Y. et al. Front. Biosci. 2008, 13, 1447-1471).

In particular, phosphorous dendrimers have elicited increasing interest from researchers during recent decades due to their numerous properties, in particular in the fields of biology and nanomedicine (Caminade A.-M. et al., Phosphorous Dendrimers in Biology and Nanomedicine: Syntheses, Characterization, and Properties, Jenny Stanford Publishing, 1st Ed., 2018)

In particular, phosphorous dendrimers with an azabisphosphonate surface (ABP dendrimer) are capable of activating monocytes and of inducing an anti-inflammatory response (WO 2010/013086 A1; Portevin D. et al. J. Transi. Med. 2009, 7, 82). More particularly, the anti-inflammatory responses have been validated in animal models of rheumatoid polyarthritis, wherein monocytes are known to perform an essential role in inflammation and osteoclastogenesis (Hayder M. et al. Sci. Trans. Med. 2011, 3, 81 ra35). The anti-inflammatory effect of the ABP dendrimer has also been validated in uveitis (Fruchon et al. Molecules 2013, 18, 9305-9316) and multiple sclerosis (Caminade A.-M. Nat. Commun. 2015, 6, 7722).

However, these phosphorous dendrimers of polyphosphorhydrazone type have, in their structure, branching points of hydrazone type which are sensitive to hydrolysis at physiological pH. Indeed, it is known that aromatic hydrazones are sensitive to hydrolysis in biological medium, in particular in plasma (Kovarikova, P. et al., J. Pharm. Biomed. Anal., 2008, 47, 360-370). It has also been demonstrated that dendrimers such as ABP having a polyphosphorhydrazone backbone and aminobismethylenephosphonic acid terminations (monosodium salt) derived from tyramine are degraded to approximately 10% after 48 hours at physiological pH and at a temperature of 37° C. (Hayder M. et al., Biomacromolecules 2018, 19, 712-720).

There is therefore a need for novel phosphorous dendrimers having good stability at physiological pH.

### SUMMARY

The present invention relates to an n generation dendrimer or the pharmaceutically acceptable salts thereof, comprising:
a central core § of valence m, where m is an integer greater than or equal to 1;
branching generation chains around the core, represented by formula (CG):

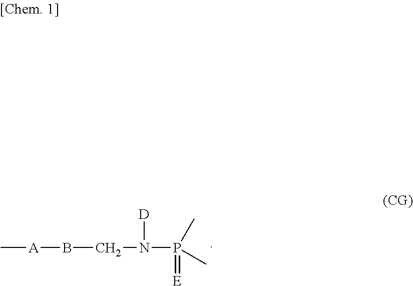

wherein
A is O or S;
B is aryl or heteroaryl, optionally substituted by C1-C6 alkyl or halo;
D is selected from C1-C12 alkyl, C1-C12 haloalkyl, aryl, heteroaryl, (CH₂—CH₂—O)ₐ—CH₃, CH₂—(CH₂—CH₂—O)ₐ—CH₃, (CH₂)_{b}—NR′R″ and CH₂—(CH₂—CH₂—O)_{c}—CH₂ —CH₂—CH₂—NR′R″, where a, b and c are, independently of one another, integers of between 1 and 12, and R′ and R″ are, independently of one another, selected from H, C1-C12 alkyl, C1-C12 haloalkyl, tert-butyloxycarbonyl, fluorenylmethoxycarbonyl, methoxycarbonyl, tert-butylcarbonyl, p-toluenesulfonyl, methylsulfonyl, trifluoromethylsulfonyl, allyl, benzyl, trityl,

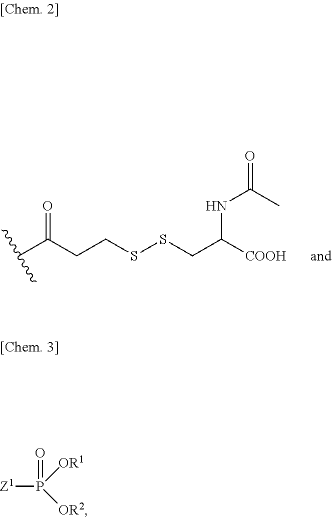

where Z¹ is a single bond or a linear or branched hydrocarbon chain having 1 to 6 chain members, one or more chain members optionally being a heteroatom, and R¹ and R² are, independently of one another, selected from H, C1-C6 alkyl and M⁺, where M⁺ is a cation;
E is O or S;
   an intermediate chain at the end of each generation chain represented by the formula (CI):

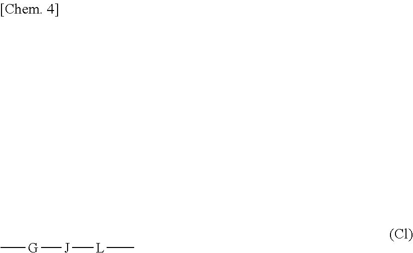

wherein
G is O or S;
J is aryl or heteroaryl, optionally substituted by C1-C6 alkyl or halo; and
L is a linear or branched hydrocarbon chain having 1 to 6 chain members, one or more chain members optionally being a heteroatom;
   an end group at the end of each intermediate chain represented by the formula (T):

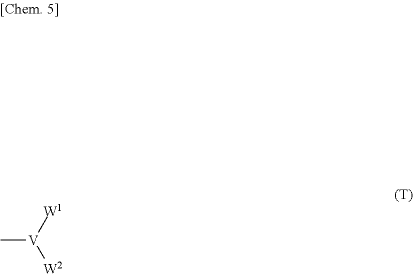

wherein
V is CH or N;
W¹ is H, C1-C12 alkyl or

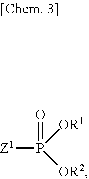

where Z¹ is a single bond or a linear or branched hydrocarbon chain having 1 to 6 chain members, one or more chain members optionally being a heteroatom, and R¹ and R² are, independently of one another, selected from H, C1-C6 alkyl and M⁺, where M⁺ is a cation; and W² is C1-C12 alkyl or

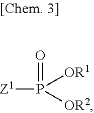

where Z¹ is a single bond or a linear or branched hydrocarbon chain having 1 to 6 chain members, one or more chain members optionally being a heteroatom, and R¹ and R² are, independently of one another, selected from H, C1-C6 alkyl and M⁺, where M⁺ is a cation;
wherein n is an integer of between 1 and 12.

According to another aspect, a method for preparing a dendrimer according to the invention is proposed, comprising the following steps, in succession:
(a) preparing an intermediate of the following formula:

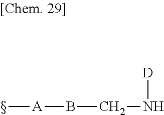

or a pharmaceutically acceptable salt thereof, wherein
§, A, B and D are as defined above;
(b) reacting the intermediate obtained in step (a) with P(E)X₃, where E is O or S and X is a halogen;
(c) reacting the product obtained in step (b) with a fragment comprising an intermediate chain and an end group.

A method for preparing a dendrimer according to the invention is also proposed, comprising the following steps, in succession:
(a) preparing a fragment comprising a generation chain, an intermediate chain and an end group of the following formula:

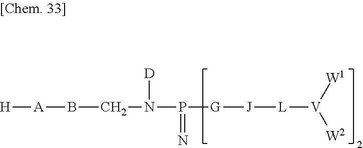

or a pharmaceutically acceptable salt thereof,
wherein
A, B, D, E, G, J, L, V, W¹ and W² are as defined above;
(b) reacting the fragment obtained in step (a) with a halogenated central core §.

According to another aspect, a pharmaceutical composition is proposed, comprising at least one dendrimer according to the invention and a pharmaceutically acceptable excipient.

Finally, the invention relates to a dendrimer according to the invention for use thereof in the treatment of inflammatory diseases.

Other characteristics, details and advantages will become apparent on reading the detailed description below.

### DETAILED DESCRIPTION OF THE INVENTION

As detailed above, the invention relates to n generation dendrimers or the pharmaceutically acceptable salts thereof, comprising:
a central core § of valence m, where m is an integer greater than or equal to 1; in particular between 1 and 12, more particularly between 1 and 8, more particularly still between 3 and 6, even more particularly, m is an integer selected from 3, 4 and 6;
branching generation chains around the core, represented by formula (CG):

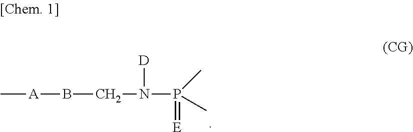

wherein
A is O or S, in particular A is O;
B is aryl or heteroaryl, optionally substituted by C1-C6 alkyl or halo, in particular B is aryl, optionally substituted by C1-C6 alkyl or halo, more particularly B is phenyl, optionally substituted by C1-C6 alkyl or halo, even more particularly B is phenyl, optionally substituted by C1-C4 alkyl, chloro or fluoro, more particularly still is phenyl;
D is selected from C1-C12 alkyl, C1-C12 haloalkyl, aryl, heteroaryl, (CH₂—CH₂—O)ₐ—CH₃, CH₂—(CH₂—CH₂—O)ₐ—CH₃, (CH₂)_{b}—NR′R″ and CH₂—(CH₂—CH₂—O)_{c}—CH₂ —CH₂—CH₂—NR′R″, where a, b and c are, independently of one another, integers of between 1 and 12, and R′ and R″ are, independently of one another, selected from H, C1-C12 alkyl, C1-C12 haloalkyl, tert-butyloxycarbonyl, fluorenylmethoxycarbonyl, methoxycarbonyl, tert-butylcarbonyl, p-toluenesulfonyl, methylsulfonyl, trifluoromethylsulfonyl, allyl, benzyl, trityl,

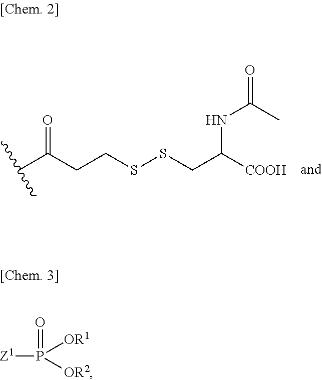

where Z¹ is a single bond or a linear or branched hydrocarbon chain having 1 to 6 chain members, one or more chain members optionally being a heteroatom, and R¹ and R² are, independently of one another, selected from H, C1-C6 alkyl and M⁺, where M⁺ is a cation; in particular D is selected from C1-C12 alkyl, C1-C12 haloalkyl, (CH₂—CH₂—O)ₐ—CH₃, CH₂—(CH₂—CH₂—O)ₐ—CH₃, (CH₂)_{b}—NR′R″ and CH₂—(CH₂—CH₂—O)_{c}—CH₂—CH₂—CH₂—NR′R″, where a, b and c are, independently of one another, integers of between 1 and 12, and R′ and R″ are, independently of one another, selected from H, C1-C12 alkyl, C1-C12 haloalkyl, tert-butyloxycarbonyl,

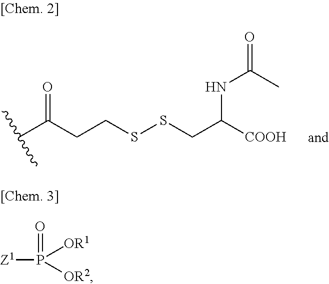

where Z¹ is a single bond or a linear or branched hydrocarbon chain having 1 to 6 chain members, and R¹ and R² are, independently of one another, selected from H, C1-C4-alkyl, Na⁺ and K⁺; more particularly, D is selected from C1-C10 alkyl, CH₂ —(CH₂—CH₂—O)ₐ—CH₃, (CH₂)_{b}—NR′R″ and CH₂—(CH₂—CH₂—O)_{c}—CH₂—CH₂—CH₂ —NR′R″, where a, b and c are, independently of one another, integers of between 1 and 10 and R′ and R″ are, independently of one another, selected from H,

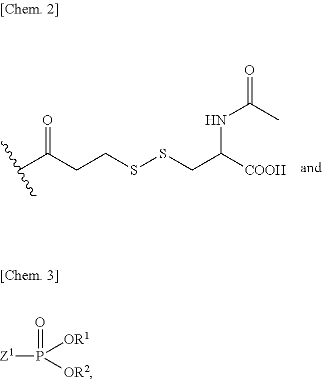

where Z¹ is a linear or branched hydrocarbon chain having 1 to 4 chain members, and R¹ and R² are, independently of one another, selected from H, C1-C2 alkyl and Na⁺; more particularly still, D is selected from C1-C8-alkyl, (CH₂)_{b}—NHR′ et CH₂—(CH₂—CH₂ —O)_{c}—CH₂—CH₂—CH₂—NHR′, where b and c are, independently of one another, integers of between 1 and 8 and R′ is H or

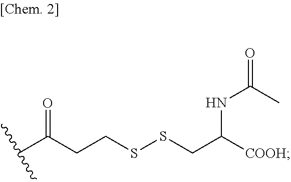 [SMILES: *C(=O)CCSSCC(NC(C)=O)C(=O)O]

E is O or S, in particular E is O;
   an intermediate chain at the end of each generation chain represented by the formula (CI):

### [Chem. 4]

-G-J-L- (CI)
wherein
G is O or S, in particular G is O;
J is aryl or heteroaryl, optionally substituted by C1-C6 alkyl or halo, in particular
J is aryl, optionally substituted by C1-C6 alkyl or halo, more particularly J is phenyl, optionally substituted by C1-C6 alkyl or halo, even more particularly J is phenyl, optionally substituted by C1-C4 alkyl, chloro or fluoro, more particularly still is phenyl,
L is a linear or branched hydrocarbon chain having 1 to 6 chain members, one or more chain members optionally being a heteroatom selected from N, O and S, in particular L is a linear or branched hydrocarbon chain having 1 to 6 chain members, one or more chain members optionally being a heteroatom selected from N and O, more particularly L is a linear or branched hydrocarbon chain having 1 to 4 chain members, more particularly still L is a linear hydrocarbon chain having 1 to 4 chain members, even more particularly L is a linear hydrocarbon chain having 1 to 2 chain members, even more particularly still L is CH₂ or CH₂—CH₂;
   an end group at the end of each intermediate chain represented by the formula (T):

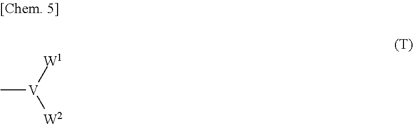

wherein
V is CH or N, in particular V is N;
W¹ is H, C1-C12 alkyl or

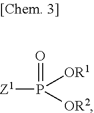

where Z¹ is a single bond or a linear or branched hydrocarbon chain having 1 to 6 chain members, one or more chain members optionally being a heteroatom, and R¹ and R² are, independently of one another, selected from H, C1-C6 alkyl and M⁺, where M⁺ is a cation; in particular W¹ is C1-C10 alkyl or

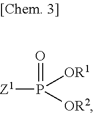

where Z¹ is a single bond or a linear or branched hydrocarbon chain having 1 to 6 chain members, and R¹ and R² are, independently of one another, selected from H, C1-C6 alkyl and M⁺, where M⁺ is a cation of an element from group IA, IIA, IIB or IIIA of the periodic table of the elements, or a cation of a nitrogenous base; more particularly, W¹ is H, C1-C8 alkyl or

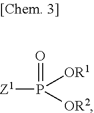

where Z¹ is a linear or branched hydrocarbon chain having 1 to 4 chain members and R¹ and R² are, independently of one another, selected from H, C1-C4 alkyl, Na⁺ and K⁺; more particularly still, W¹ is H, C1-C6 alkyl or [Chem. 3]

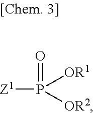

where Z¹ is a linear hydrocarbon chain having 1 to 2 chain members and R¹ and R² are, independently of one another, selected from H, C1-C2 alkyl and Na⁺;
W² is C1-C12 alkyl or

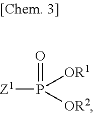

where Z¹ is a single bond or a linear or branched hydrocarbon chain having 1 to 6 chain members, one or more chain members optionally being a heteroatom, and R¹ and R² are, independently of one another, selected from H, C1-C6 alkyl and M⁺, where M⁺ is a cation; in particular W² is C1-C10 alkyl or

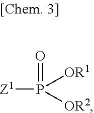

where Z¹ is a single bond or a linear or branched hydrocarbon chain having 1 to 6 chain members, and R¹ and R² are, independently of one another, selected from H, C1-C6 alkyl and M⁺, where M⁺ is a cation of an element from group IA, IIA, IIB or IIIA of the periodic table of the elements, or a cation of a nitrogenous base; more particularly, W² is C1-C8 alkyl or

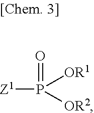

where Z¹ is a linear or branched hydrocarbon chain having 1 to 4 chain members and R¹ and R² are, independently of one another, selected from H, C1-C4 alkyl, Na⁺ and K⁺; more particularly still, W¹ is C1-C6 alkyl or

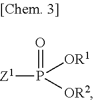

where Z¹ is a linear hydrocarbon chain having 1 to 2 chain members and R¹ and R² are, independently of one another, selected from H, C1-C2 alkyl and Na⁺;
wherein n is an integer between 1 and 12, in particular n is an integer of between 1 and 10, more particularly n is an integer of between 1 and 8, more particularly still n is an integer of between 1 and 6, even more particularly n is an integer of between 1 and 5.

Thus, the dendrimer of the invention, or the pharmaceutically acceptable salts thereof, comprises intermediate chains of formula (CI) terminated by an end group of formula (T) at the end of the generation chain of formula (CG).

The dendrimer of the invention, or the pharmaceutically acceptable salts thereof, thus comprises m arms connected to the central core §, each of these arms consisting of one or more generation chains of formula (CG) comprising, at the ends thereof, an intermediate chain of formula (CI) terminated by an end group of formula (T).

It is to the inventors' credit to have demonstrated that the presence of generation chains of formula (CG) as defined above within the dendrimer made it possible to improve the stability of the dendrimer at physiological pH.

In one embodiment, the central core § present in the dendrimer of the invention is selected from pentoses, hexoses, and groups of the following formulae:

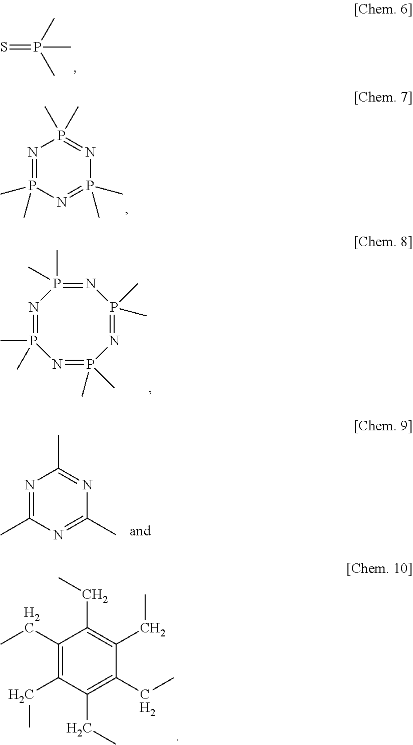

In particular, the central core § present in the dendrimer of the invention is selected from the groups of the following formulae:

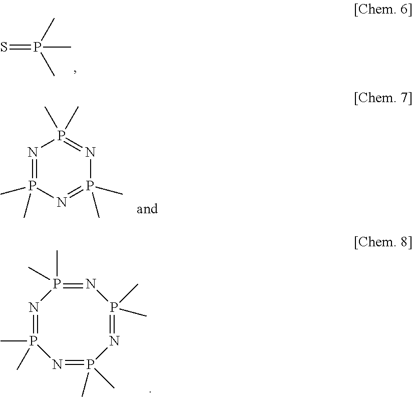

More particularly, the central core § present in the dendrimer of the invention is the group of the following formula:

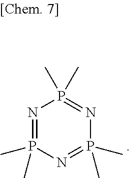

In one embodiment, the dendrimer of the invention is characterized in that m is an integer of between 1 and 8. In particular, the dendrimer of the invention is characterized in that m is an integer selected form 3, 4 and 6. More particularly, the dendrimer of the invention is characterized in that m is equal to 6.

In one embodiment, the dendrimer of the invention is characterized in that n is an integer of between 1 and 5.

In one embodiment, the dendrimer of the invention is characterized in that, in formula (CG), A is O.

In one embodiment, the dendrimer of the invention is characterized in that, in formula (CG), B is phenyl, optionally substituted by C1-C6 alkyl or halo, in particular B is phenyl, optionally substituted by C1-C4 alkyl, chloro or fluoro, more particularly, B is phenyl.

In one embodiment, the dendrimer of the invention is characterized in that, in formula (CG), E is S.

In one embodiment, the dendrimer of the invention is characterized in that, in formula (CI), G is O.

In one embodiment, the dendrimer of the invention is characterized in that, in formula (CI), J is phenyl, optionally substituted by C1-C6 alkyl or halo, in particular J is phenyl, optionally substituted by C1-C4 alkyl, chloro or fluoro, more particularly, J is phenyl.

In one embodiment, the dendrimer of the invention is characterized in that, in formula (CI), L is (CH₂)_{d}, where D is an integer of between 1 and 6, d is an integer of between 1 and 4, more particularly d is an integer of between 1 and 2.

In one embodiment, the dendrimer of the invention is characterized in that, in formula (T), V is N.

In one embodiment, the dendrimer of the invention is characterized in that, in formula (T), W¹ is H and W² is C1-C12 alkyl.

In one embodiment, the dendrimer of the invention is characterized in that, in formula (T), W¹ and W² are

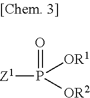

where Z¹ is a single bond or a linear or branched hydrocarbon chain having 1 to 6 chain members, and R¹ and R² are, independently of one another, selected from H, C1-C6 alkyl and M⁺, where M⁺ is a cation of an element from group IA, IIA, IIB or IIIA of the periodic table of the elements, or a cation of a nitrogenous base. In particular, M is selected from atoms of sodium, potassium, calcium, barium, zinc, magnesium, lithium and aluminum. More particularly, M⁺ is selected from Na⁺, K⁺ and Li⁺. Even more particularly, M⁺ is Na⁺ or K⁺.

In one embodiment, the dendrimer of the invention is characterized in that, in formula (T), W¹ and W² are

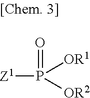

where Z¹ is CH₂ and R¹ and R²are, independently of one another, selected from H, methyl and Na⁺.

In other words, the invention relates to a dendrimer of formula (I):

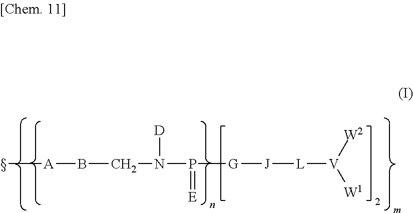

or the pharmaceutically acceptable salts thereof,
wherein
A is O or S, in particular A is O;
B is aryl or heteroaryl, optionally substituted by C1-C6 alkyl or halo, in particular B is aryl, optionally substituted by C1-C6 alkyl or halo, more particularly B is phenyl, optionally substituted by C1-C6 alkyl or halo, even more particularly B is phenyl, optionally substituted by C1-C4 alkyl, chloro or fluoro, more particularly still is phenyl;
D is selected from C1-C12 alkyl, C1-C12 haloalkyl, aryl, heteroaryl, (CH₂—CH₂—O)ₐ—CH₃, CH₂—(CH₂—CH₂—O)ₐ—CH₃, (CH₂)_{b}—NR′R″ and CH₂—(CH₂—CH₂—O)_{c}—CH₂—CH₂—CH₂—NR′R″, where a, b and c are, independently of one another, integers of between 1 and 12, and R′ and R″ are, independently of one another, selected from H, C1-C12 alkyl, C1-C12 haloalkyl, tert-butyloxycarbonyl, fluorenylmethoxycarbonyl, methoxycarbonyl, tert-butylcarbonyl, p-toluenesulfonyl, methylsulfonyl, trifluoromethylsulfonyl, allyl, benzyl, trityl,

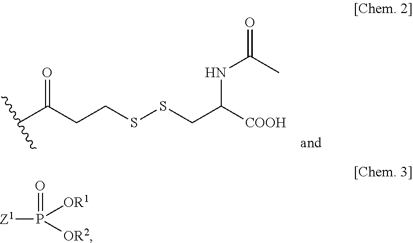

where Z¹ is a single bond or a linear or branched hydrocarbon chain having 1 to 6 chain members, one or more chain members optionally being a heteroatom, and R¹ and R² are, independently of one another, selected from H, C1-C6 alkyl and M⁺, where M⁺ is a cation; in particular D is selected from C1-C12 alkyl, C1-C12 haloalkyl, (CH₂—CH₂—O)ₐ—CH₃, CH₂—(CH₂—CH₂—O)ₐ—CH₃, (CH₂)_{b}—NR′R″ and CH₂—(CH₂—CH₂—O)_{c}—CH₂—CH₂—CH₂—NR′R″, where a, b and c are, independently of one another, integers of between 1 and 12, and R′ and R″ are, independently of one another, selected from H, C1-C12 alkyl, C1-C12 haloalkyl, tert-butyloxycarbonyl,

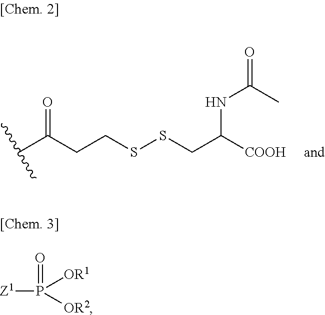

where Z¹ is a single bond or a linear or branched hydrocarbon chain having 1 to 6 chain members, and R¹ and R² are, independently of one another, selected from H, C1-C4-alkyl, Na⁺ and K⁺; more particularly, D is selected from C1-C10 alkyl, CH₂ —(CH₂—CH₂—O)ₐ—CH₃, (CH₂)_{b}—NR′R″ and CH₂—(CH₂—CH₂—O)_{c}—CH₂—CH₂—CH₂ —NR′R″, where a, b and c are, independently of one another, integers of between 1 and 10 and R′ and R″ are, independently of one another, selected from H,

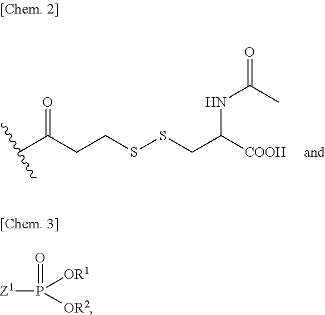

where Z¹ is a linear or branched hydrocarbon chain having 1 to 4 chain members, and R¹ and R² are, independently of one another, selected from H, C1-C2 alkyl and Na⁺; more particularly still, D is selected from C1-C8-alkyl, (CH₂)_{b}—NHR′ et CH₂—(CH₂—CH₂ —O)_{c}—CH₂—CH₂—CH₂—NHR′, where b and c are, independently of one another, integers of between 1 and 8 and R′ is H or

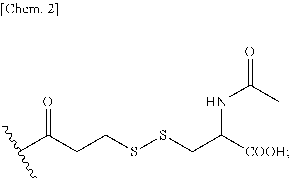 [SMILES: *C(=O)CCSSCC(NC(C)=O)C(=O)O]

E is O or S, in particular E is O;
G is O or S, in particular G is O;
J is aryl or heteroaryl, optionally substituted by C1-C6 alkyl or halo, in particular J is aryl, optionally substituted by C1-C6 alkyl or halo, more particularly J is phenyl, optionally substituted by C1-C6 alkyl or halo, even more particularly J is phenyl, optionally substituted by C1-C4 alkyl, chloro or fluoro, more particularly still J is phenyl,
L is a linear or branched hydrocarbon chain having 1 to 6 chain members, one or more chain members optionally being a heteroatom selected from N, O and S, in particular L is a linear or branched hydrocarbon chain having 1 to 6 chain members, one or more chain members optionally being a heteroatom selected from N and O, more particularly L is a linear or branched hydrocarbon chain having 1 to 4 chain members, more particularly still L is a linear hydrocarbon chain having 1 to 4 chain members, even more particularly L is a linear hydrocarbon chain having 1 to 2 chain members, even more particularly still L is CH₂ or CH₂—CH₂;
V is CH or N, in particular V is N;
W¹ is H, C1-C12 alkyl or

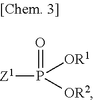

where Z¹ is a single bond or a linear or branched hydrocarbon chain having 1 to 6 chain members, one or more chain members optionally being a heteroatom, and R¹ and R² are, independently of one another, selected from H, C1-C6 alkyl and M⁺, where M⁺ is a cation; in particular W¹ is C1-C10 alkyl or

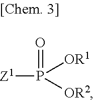

where Z¹ is a single bond or a linear or branched hydrocarbon chain having 1 to 6 chain members, and R¹ and R² are, independently of one another, selected from H, C1-C6 alkyl and M⁺, where M⁺ is a cation of an element from group IA, IIA, IIB or IIIA of the periodic table of the elements, or a cation of a nitrogenous base; more particularly, W¹ is H, C1-C8 alkyl or

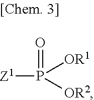

where Z¹ is a linear or branched hydrocarbon chain having 1 to 4 chain members and R¹ and R² are, independently of one another, selected from H, C1-C4 alkyl, Na⁺ and K⁺; more particularly still, W¹ is H, C1-C6 alkyl or [Chem. 3]

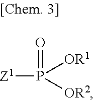

where Z¹ is a linear hydrocarbon chain having 1 to 2 chain members and R¹ and R² are, independently of one another, selected from H, C1-C2 alkyl and Na⁺;
W² is C1-C12 alkyl or

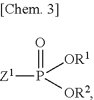

where Z¹ is a single bond or a linear or branched hydrocarbon chain having 1 to 6 chain members, one or more chain members optionally being a heteroatom, and R¹ and R² are, independently of one another, selected from H, C1-C6 alkyl and M⁺, where M⁺ is a cation; in particular W² is C1-C10 alkyl or

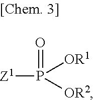

where Z¹ is a single bond or a linear or branched hydrocarbon chain having 1 to 6 chain members, and R¹ and R² are, independently of one another, selected from H, C1-C6 alkyl and M⁺, where M⁺ is a cation of an element from group IA, IIA, IIB or IIIA of the periodic table of the elements, or a cation of a nitrogenous base; more particularly, W² is C1-C8 alkyl or

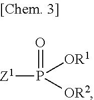

where Z¹ is a linear or branched hydrocarbon chain having 1 to 4 chain members and R¹ and R² are, independently of one another, selected from H, C1-C4 alkyl, Na⁺ and K⁺; more particularly still, W² is C1-C6 alkyl or

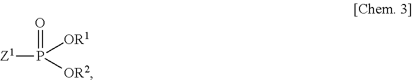

where Z¹ is a linear hydrocarbon chain having 1 to 2 chain members and R¹ and R² are, independently of one another, selected from H, C1-C2 alkyl and Na⁺;
m is an integer between 1 and 8, in particular, m is an integer selected among 3, 4 and 6, more particularly, m is equal to 6;
n is an integer between 1 and 12, in particular n is an integer between 1 and 10, more particularly n is an integer between 1 and 8, more particularly n is an integer between 1 and 6, still more particularly n is an integer between 1 and 5; and
§ is selected from the pentoses, hexoses and groups of the following formulae:

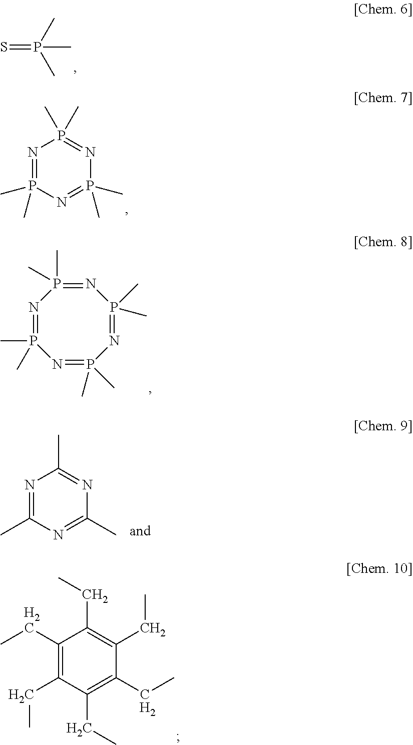

in particular, § is selected from the groups of the following formulae:

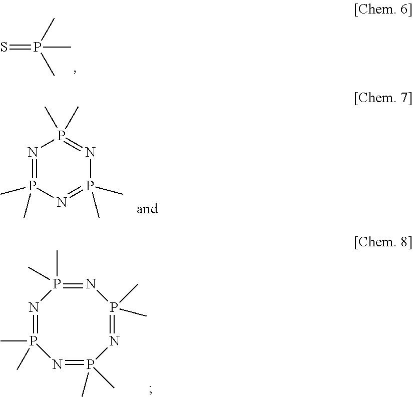

more particularly, § is the group of the following formula:

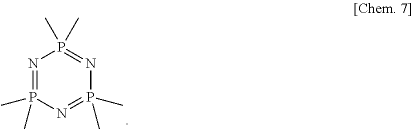

According to a first variant, the dendrimer of the invention is characterized in that, in formula (T), V is N and W¹ is H.

In this case, the dendrimer according to the invention may be represented by the following formula (II):

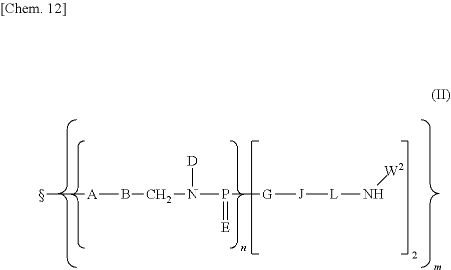

or the pharmaceutically acceptable salts thereof,
wherein
§, A, B, D, E, G, J, L, W², m and n are as defined previously or in formula (I), and
{ }ₙ denotes the branching structure of the n generation chains of said dendrimer.

Preferred dendrimers of formula (II) are those wherein §, A, B, D, E, G, J, L, W², m and n are defined as follows:
§ is selected from the groups of the following formulae:

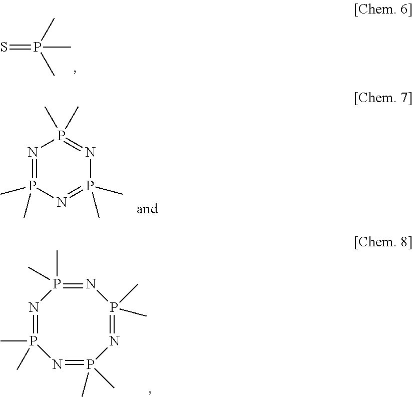

in particular, § is the group of the following formula:

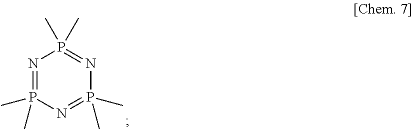

A is O or S, in particular A is O;
B is aryl or heteroaryl, optionally substituted by C1-C6 alkyl or halo, in particular B is aryl, optionally substituted by C1-C6 alkyl or halo, more particularly B is phenyl, optionally substituted by C1-C6 alkyl or halo, even more particularly B is phenyl, optionally substituted by C1-C4 alkyl, chloro or fluoro, more particularly still B is phenyl;
D is selected from C1-C12 alkyl, C1-C12 haloalkyl, (CH₂—CH₂—O)ₐ—CH₃, CH₂—(CH₂—CH₂—O)ₐ—CH₃, (CH₂)_{b}—NR′R″ and CH₂—(CH₂—CH₂—O)_{c}—CH₂—CH₂—CH₂—NR′R″, where a, b and c are, independently of one another, integers of between 1 and 12 and R′ and R″ are, independently of one another, selected from H, C1-C12 alkyl, C1-C12 haloalkyl, tert-butyloxycarbonyl,

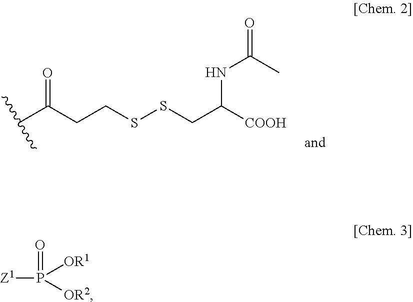

where Z¹ is a single bond or a linear or branched hydrocarbon chain having 1 to 6 chain members, and R¹ and R² are, independently of one another, selected from H, C1-C4-alkyl, Na⁺ and K⁺; in particular, D is selected from C1-C10 alkyl, CH₂ —(CH₂—CH₂—O)ₐ—CH₃, (CH₂)_{b}—NHR′ and CH₂—(CH₂—CH₂—O)_{c}—CH₂—CH₂—CH₂ —NHR′R″, where a, b and c are, independently of one another, integers of between 1 and 10 and R′ is H or

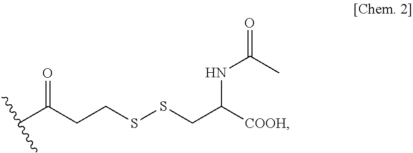 [SMILES: *C(=O)CCSSCC(NC(C)=O)C(=O)O]

more particularly, D is selected from C1-C8-alkyl and CH₂—(CH₂—CH₂—O)ₐ—CH₃, where a is an integer of between 1 and 8; more particularly still, D is C1-C8 alkyl, even more particularly, D is selected from methyl, hexyl and octyl; most particularly, D is methyl;
E is O or S, in particular E is S;
G is O or S, in particular G is O;
J is aryl or heteroaryl, optionally substituted by C1-C6 alkyl or halo, in particular J is aryl, optionally substituted by C1-C6 alkyl or halo, more particularly J is phenyl, optionally substituted by C1-C6 alkyl or halo, even more particularly J is phenyl, optionally substituted by C1-C4 alkyl, chloro or fluoro, more particularly still is phenyl,
L is a linear or branched hydrocarbon chain having 1 to 6 chain members, each chain member optionally being a heteroatom, in particular L is a linear or branched hydrocarbon chain having 1 to 6 chain members, more particularly L is a linear or branched hydrocarbon chain having 1 to 4 chain members, more particularly still L is a linear hydrocarbon chain having 1 to 4 chain members, even more particularly L is a linear hydrocarbon chain having 1 to 2 chain members, even more particularly still L is CH₂;
W² is C1-C12 alkyl, in particular W² is C1-C10 alkyl, more particularly W² is C1-C8 alkyl, more particularly still W² is C1-C4 alkyl, even more particularly W² is methyl;
m is an integer greater than or equal to 1, in particular m is an integer between 1 and 12, more particularly m is an integer between 1 and 8, still more particularly m is an integer between 3 and 6, still more particularly m is an integer chosen from 3, 4 and 6;
n is an integer between 1 and 12, in particular n is an integer between 1 and 10, more particularly n is an integer between 1 and 8, more particularly n is an integer between 1 and 6, still more particularly n is an integer between 1 and 5.

According to a particular embodiment, the dendrimer of the invention is characterized in that n is equal to 1. In this case, the dendrimer of the invention is that of formula (II₁):

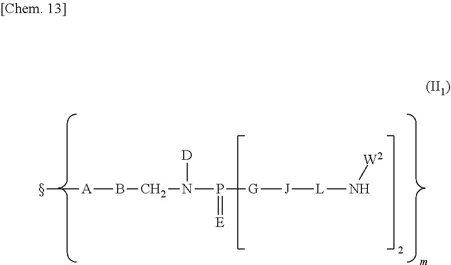

or the pharmaceutically acceptable salts thereof,
wherein
§, A, B, D, E, G, J, L, W², and m are as defined in formula (II).

According to a particular embodiment, the dendrimer of the invention is characterized in that n is equal to 2. In this case, the dendrimer of the invention is that of formula (II₂):

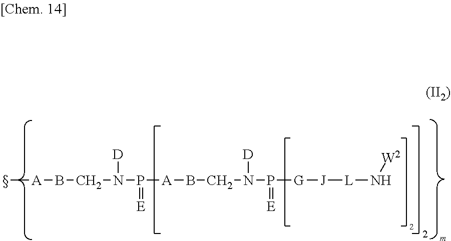

or the pharmaceutically acceptable salts thereof,
wherein
§, A, B, D, E, G, J, L, W², and m are as defined in formula (II).

According to a particular embodiment, the dendrimer of the invention is characterized in that n is equal to 3. In this case, the dendrimer of the invention is that of formula (II₃):

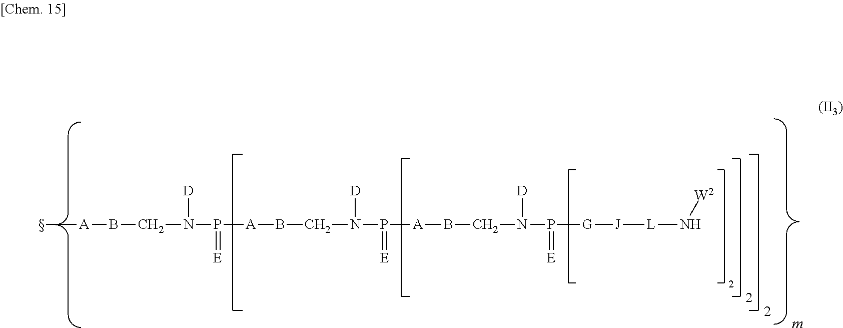

or the pharmaceutically acceptable salts thereof,
wherein
§, A, B, D, E, G, J, L, W², and m are as defined in formula (II).

According to a particular embodiment, the dendrimer of the invention is characterized in that n is equal to 4. In this case, the dendrimer of the invention is that of formula (II₄):

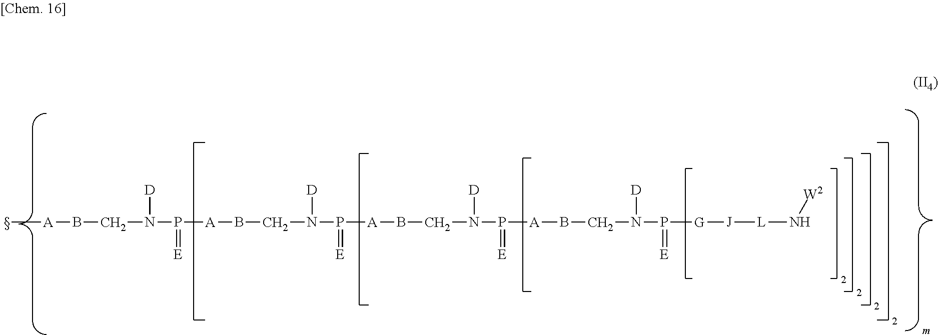

or the pharmaceutically acceptable salts thereof,
wherein
§, A, B, D, E, G, J, L, W², and m are as defined in formula (II).

According to a particular embodiment, the dendrimer of the invention is characterized in that n is equal to 5. In this case, the dendrimer of the invention is that of formula (II₅):

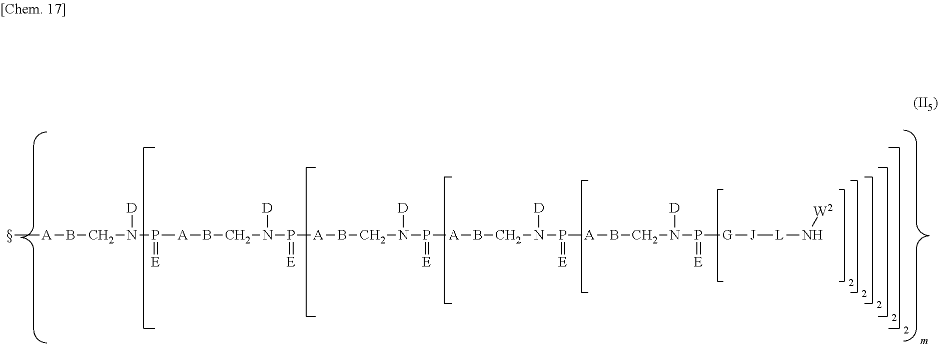

or the pharmaceutically acceptable salts thereof,
wherein
§, A, B, D, E, G, J, L, W², and m are as defined in formula (II).

Preferred dendrimers of formula (II) are those of formula (IIA):

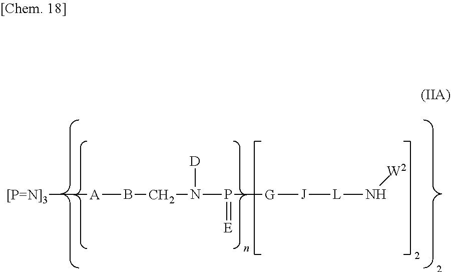

or the pharmaceutically acceptable salts thereof,
wherein
A, B, D, E, G, J, L, W², and n are as defined in relation to formula (II),
{ }ₙ denotes the branching structure of the n generation chains of said dendrimer, and
[P═N]₃ denotes the central core of the following formula:

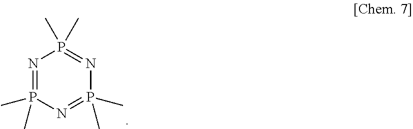

Preferred dendrimers of formula (IIA) are those wherein A, B, D, E, G, J, L, W² and n are defined as follows:
A is O or S, in particular A is O;
B is phenyl, optionally substituted by C1-C6 alkyl or halo, in particular B is phenyl;
D is C1-C12 alkyl, in particular D is C1-C10-alkyl, more particularly D is C1-C8 alkyl, more particularly still D is selected from methyl, hexyl and octyl, even more particularly D is methyl;
E is O or S, in particular E is S;
G is O or S, in particular G is O;
J is phenyl, optionally substituted by C1-C6 alkyl or halo, in particular J is phenyl;
L is a linear or branched hydrocarbon chain having 1 to 4 chain members, in particular L is a linear hydrocarbon chain having 1 to 4 chain members, more particularly L is a linear hydrocarbon chain having 1 to 2 chain members, even more particularly L is CH₂;
W² is C1-C8 alkyl, more particularly still W² is C1-C4 alkyl, even more particularly W² is methyl;
n is an integer of between 1 and 6, in particular n is an integer of between 1 and 5.

Other preferred dendrimers of formula (II) are those of formula (IIB):

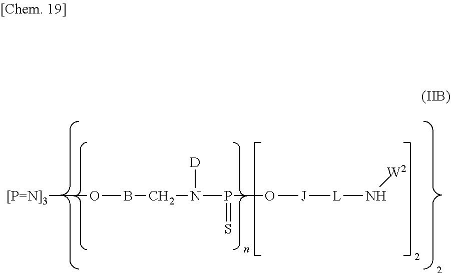

or the pharmaceutically acceptable salts thereof,
wherein
B, D, J, L, W² and n are as defined in relation to formula (II),
{ }ₙ denotes the branching structure of the n generation chains of said dendrimer, and
[P═N]₃ denotes the central core of the following formula:

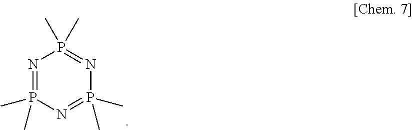

Preferred dendrimers of formula (IIB) are those wherein B, D, J, L, W² and n are defined as follows:
B is phenyl, optionally substituted by C1-C6 alkyl or halo, in particular B is phenyl;
D is C1-C12 alkyl, in particular D is C1-C10-alkyl, more particularly D is C1-C8 alkyl, more particularly still D is selected from methyl, hexyl and octyl, even more particularly D is methyl;
J is phenyl, optionally substituted by C1-C6 alkyl or halo, in particular J is phenyl;
L is a linear or branched hydrocarbon chain having 1 to 4 chain members, in particular L is a linear hydrocarbon chain having 1 to 4 chain members, more particularly L is a linear hydrocarbon chain having 1 to 2 chain members, even more particularly L is CH₂;
W² is C1-C8 alkyl, more particularly still W² is C1-C4 alkyl, even more particularly W² is methyl;
n is an integer of between 1 and 6, in particular n is an integer of between 1 and 5.

Other preferred dendrimers of formula (II) are those of formula (IIC):

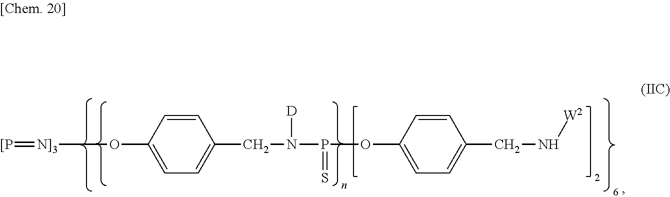

or the pharmaceutically acceptable salts thereof,
wherein
D, W² and n are as defined in relation to formula (II),
{ }ₙ denotes the branching structure of the n generation chains of said dendrimer, and
[P═N]₃ denotes the central core of the following formula:

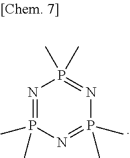

Preferred dendrimers of formula (IIC) are those wherein D, W² and n are defined as follows:
D is C1-C12 alkyl, in particular D is C1-C10-alkyl, more particularly D is C1-C8 alkyl, more particularly still D is selected from methyl, hexyl and octyl, even more particularly D is methyl;
W² is C1-C8 alkyl, more particularly still W² is C1-C4 alkyl, even more particularly W² is methyl;
n is an integer of between 1 and 6, in particular n is an integer of between 1 and 5.

According to a second variant, the dendrimer of the invention is characterized in that, in formula (T), W¹ and W² are

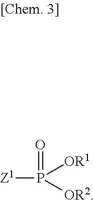

In this case, the dendrimer according to the invention may be represented by the following formula (III):

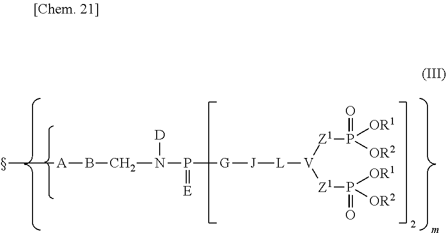

or the pharmaceutically acceptable salts thereof,
wherein
§, A, B, D, E, G, J, L, V, Z¹, R¹, R², m and n are as defined previously or in formula (I), and
{ }ₙ denotes the branching structure of the n generation chains of said dendrimer.

Preferred dendrimers of formula (III) are those wherein §, A, B, D, E, G, J, L, V, Z¹, R¹, R², m and n are defined as follows:
§ is selected from the groups of the following formulae:

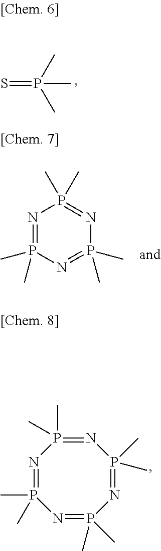

in particular, § is the group of the following formula:

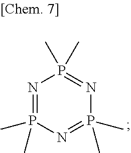

A is O or S, in particular A is O;
B is aryl or heteroaryl, optionally substituted by C1-C6 alkyl or halo, in particular B is aryl, optionally substituted by C1-C6 alkyl or halo, more particularly B is phenyl, optionally substituted by C1-C6 alkyl or halo, even more particularly B is phenyl, optionally substituted by C1-C4 alkyl, chloro or fluoro, more particularly still B is phenyl;
D is selected from C1-C12 alkyl, (CH₂—CH₂—O)ₐ—CH₃, CH₂—(CH₂—CH₂—O)ₐ—CH₃, (CH₂)_{b}—NHR′ and CH₂—(CH₂—CH₂—O)_{c}—CH₂—CH₂—CH₂—NHR, where a, b and c are, independently of one another, integers of between 1 and 12 and R′ is H or

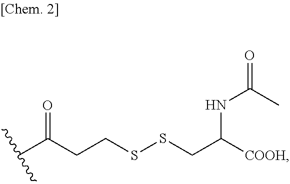 [SMILES: *C(=O)CCSSCC(NC(C)=O)C(=O)O]

in particular D is selected from C1-C10 alkyl, (CH₂—CH₂—O)ₐ—CH₃, CH₂—(CH₂—CH₂—O)ₐ—CH₃, (CH₂)_{b}—NHR′ and CH₂—(CH₂—CH₂—O)_{c}—CH₂—CH₂—CH₂—NHR′, where a, b and c are, independently of one another, integers of between 1 and 10 and R′ is H or

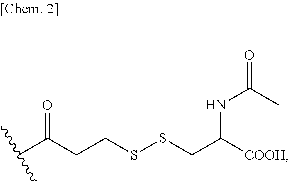 [SMILES: *C(=O)CCSSCC(NC(C)=O)C(=O)O]

more particularly D is selected from C1-C8 alkyl, CH₂—(CH₂—CH₂—O)ₐ—CH₃, (CH₂)_{b}—NHR′ and CH₂—(CH₂—CH₂—O)_{c}—CH₂—CH₂—CH₂—NHR′, where a, b and c are, independently of one another, integers of between 1 and 8 and R′ is H or

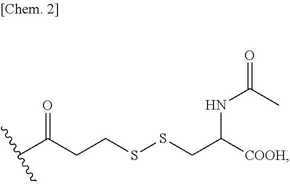 [SMILES: *C(=O)CCSSCC(NC(C)=O)C(=O)O]

more particularly still D is selected from C1-C8 alkyl, (CH₂)_{b}—NHR′ et CH₂—(CH₂—CH₂—O)_{c}—CH₂—CH₂—CH₂—NHR′, where b and c are, independently of one another, integers of between 1 and 6 and R′ is H or

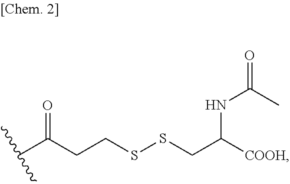 [SMILES: *C(=O)CCSSCC(NC(C)=O)C(=O)O]

even more particularly still D is selected from methyl, hexyl, octyl, (CH₂)₂ —NHR′ and CH₂—(CH₂—CH₂—O)₃—CH₂—CH₂—CH₂—NHR′, where R′ is

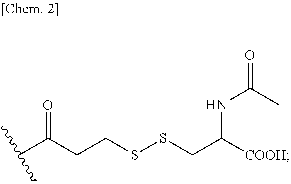 [SMILES: *C(=O)CCSSCC(NC(C)=O)C(=O)O]

E is O or S, in particular E is S;
G is O or S, in particular G is O;
J is aryl or heteroaryl, optionally substituted by C1-C6 alkyl or halo, in particular J is aryl, optionally substituted by C1-C6 alkyl or halo, more particularly J is phenyl, optionally substituted by C1-C6 alkyl or halo, even more particularly J is phenyl, optionally substituted by C1-C4 alkyl, chloro or fluoro, more particularly still is phenyl,
L is a linear or branched hydrocarbon chain having 1 to 6 chain members, each chain member optionally being a heteroatom, in particular L is a linear or branched hydrocarbon chain having 1 to 6 chain members, more particularly L is a linear or branched hydrocarbon chain having 1 to 4 chain members, more particularly still L is a linear hydrocarbon chain having 1 to 4 chain members, even more particularly L is a linear hydrocarbon chain having 1 to 2 chain members, even more particularly still L is CH₂—CH₂;
V is CH or N, in particular V is N;
Z¹ is a single bond or a linear or branched hydrocarbon chain having 1 to 6 chain members, each chain member optionally being a heteroatom, in particular Z¹ is a single bond or a linear or branched hydrocarbon chain having 1 to 6 chain members, more particularly Z¹ is a single bond or a linear or branched hydrocarbon chain having 1 to 4 chain members, more particularly still Z¹ s a linear hydrocarbon chain having 1 to 4 chain members, even more particularly Z¹ is a linear hydrocarbon chain having 1 to 2 chain members;
R¹ and R² are, independently of one another, selected from H, C1-C6 alkyl and M⁺, where M⁺ is a cation, in particular R¹ and R² are, independently of one another, selected from H, C1-C4 alkyl and M⁺, where M⁺ is a cation of an element of group IA, IIA, IIB or IIIIA of the periodic table of the elements or a cation of a nitrogenous base, more particularly R¹ and R² are, independently of one another, selected from H, C1-C4 alkyl, Na⁺ and K⁺;
m is an integer greater than or equal to 1, in particular m is an integer between 1 and 12, more particularly m is an integer between 1 and 8, still more particularly m is an integer between 3 and 6, still more particularly m is an integer chosen from 3, 4 and 6;
n is an integer between 1 and 12, in particular n is an integer between 1 and 10, more particularly n is an integer between 1 and 8, more particularly n is an integer between 1 and 6, still more particularly n is an integer between 1 and 5.

According to a particular embodiment, the dendrimer of the invention is characterized in that n is equal to 1. In this case, the dendrimer of the invention is that of formula (III₁):

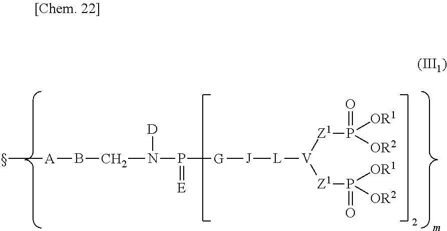

or the pharmaceutically acceptable salts thereof,
wherein
§, A, B, D, E, G, J, L, V, Z¹, R¹, R², and m are as defined in formula (III).

According to a particular embodiment, the dendrimer of the invention is characterized in that n is equal to 2. In this case, the dendrimer of the invention is that of formula (III₂):

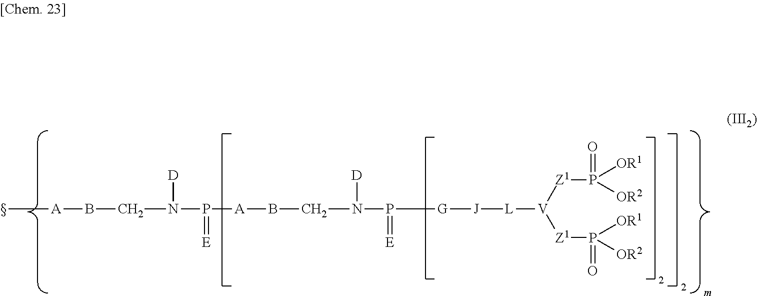

or the pharmaceutically acceptable salts thereof,
wherein
§, A, B, D, E, G, J, L, V, Z¹, R¹, R², and m are as defined in formula (III).

According to a particular embodiment, the dendrimer of the invention is characterized in that n is equal to 3. In this case, the dendrimer of the invention is that of formula (III₃):

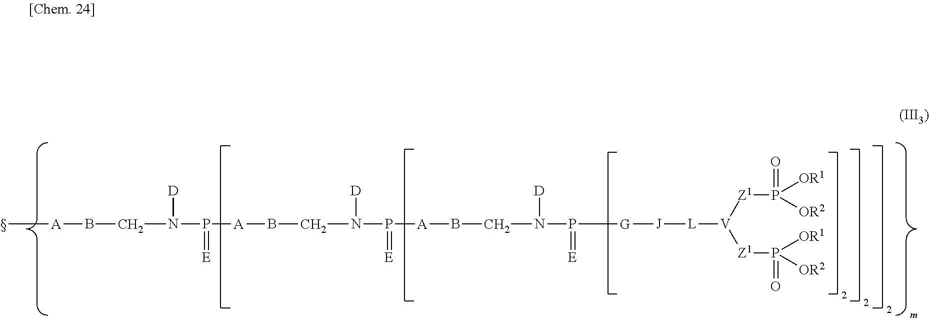

or the pharmaceutically acceptable salts thereof,
wherein
§, A, B, D, E, G, J, L, V, Z¹, R¹, R², and m are as defined in formula (III).

According to a particular embodiment, the dendrimer of the invention is characterized in that n is equal to 4.

According to a particular embodiment, the dendrimer of the invention is characterized in that n is equal to 5.

According to a particular embodiment, the dendrimer of the invention is bifunctional. In this case, the dendrimer comprises a functional group at the generation chain and one or more functional groups at the end group.

Bifunctional dendrimers are those of formula (III) wherein D is defined as follows:
D is (CH₂)_{b}—NHR′ ou CH₂—(CH₂—CH₂—O)_{c}—CH₂—CH₂—CH₂—NHR′, where b and c are, independently of one another, integers of between 1 and 12 and R′ is

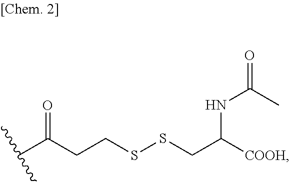 [SMILES: *C(=O)CCSSCC(NC(C)=O)C(=O)O]

in particular D is (CH₂)_{b}—NHR′ ou CH₂—(CH₂—CH₂—O)_{c}—CH₂—CH₂—CH₂—NHR′, where b and c are, independently of one another, integers of between 1 and 10 and R′ is

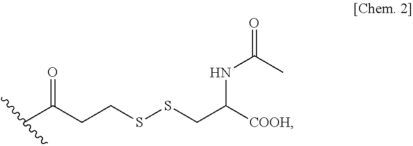 [SMILES: *C(=O)CCSSCC(NC(C)=O)C(=O)O]

more particularly, D is (CH₂)_{b}—NHR′ ou CH₂—(CH₂—CH₂—O)_{c}—CH₂—CH₂—CH₂—NHR′, where b and c are, independently of one another, integers of between 1 and 8 and R′ is

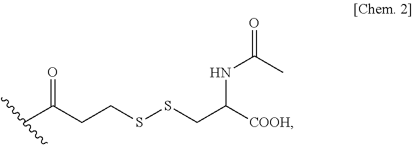 [SMILES: *C(=O)CCSSCC(NC(C)=O)C(=O)O]

Preferred dendrimers of formula (III) are those of formula (IIIA):

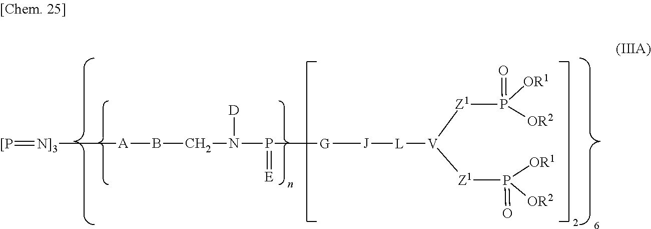

or the pharmaceutically acceptable salts thereof,
wherein
A, B, D, E, G, J, L, V, Z¹, R¹, R², and n are as defined in relation to formula (III),
{ }ₙ denotes the branching structure of the n generation chains of said dendrimer, and
[P═N]₃ denotes the central core of the following formula:

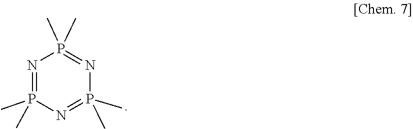

Preferred dendrimers of formula (IIIA) are those wherein A, B, D, E, G,
J, L, V, Z¹, R¹, R², and n are defined as follows:
A is O or S, in particular A is O;
B is phenyl, optionally substituted by C1-C6 alkyl or halo, in particular B is phenyl;
D is selected from C1-C12 alkyl, CH₂—(CH₂—CH₂—O)ₐ—CH₃, (CH₂)_{b}—NHR′ and CH₂—(CH₂—CH₂—O)_{c}—CH₂—CH₂—CH₂—NHR′, where a, b and c are, independently of one another, integers of between 1 and 12 and R′ is H or

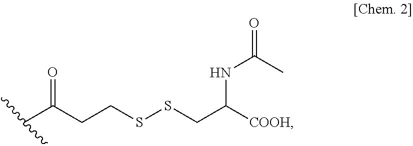 [SMILES: *C(=O)CCSSCC(NC(C)=O)C(=O)O]

in particular D is selected from C1-C10 alkyl, CH₂—(CH₂—CH₂—O)ₐ—CH₃, (CH₂)_{b}—NHR′ and CH₂—(CH₂—CH₂—O)_{c}—CH₂—CH₂—CH₂—NHR′, where a, b and c are, independently of one another, integers of between 1 and 10 and R′ is H or

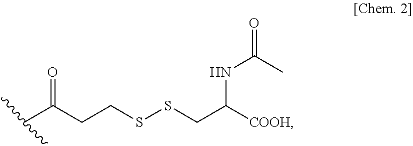 [SMILES: *C(=O)CCSSCC(NC(C)=O)C(=O)O]

more particularly D is selected from C1-C8 alkyl, CH₂—(CH₂—CH₂—O)ₐ—CH₃, (CH₂)_{b}—NHR′ and CH₂—(CH₂—CH₂—O)_{c}—CH₂—CH₂—CH₂—NHR′, where a, b and c are, independently of one another, integers of between 1 and 8 and R′ is H or

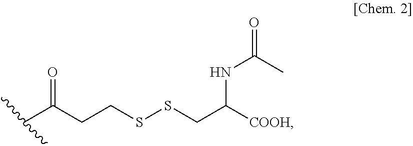 [SMILES: *C(=O)CCSSCC(NC(C)=O)C(=O)O]

more particularly still D is selected from C1-C8 alkyl, (CH₂)_{b}—NHR′ et CH₂—(CH₂—CH₂—O)_{c}—CH₂—CH₂—CH₂—NHR′, where b and c are, independently of one another, integers of between 1 and 6 and R′ is H or

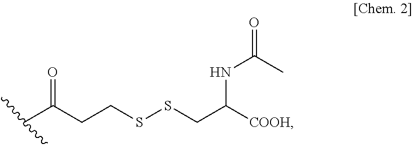 [SMILES: *C(=O)CCSSCC(NC(C)=O)C(=O)O]

even more particularly still D is selected from methyl, hexyl, octyl, (CH₂)₂ —NHR′ and CH₂—(CH₂—CH₂—O)₃—CH₂—CH₂—CH₂—NHR′, where R′ is

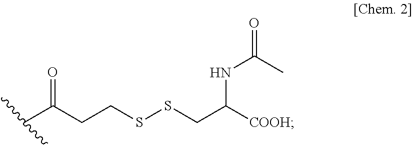 [SMILES: *C(=O)CCSSCC(NC(C)=O)C(=O)O]

E is O or S, in particular E is S;
G is O or S, in particular G is O;
J is phenyl, optionally substituted by C1-C6 alkyl or halo, in particular J is phenyl;
L is a linear or branched hydrocarbon chain having 1 to 4 chain members, in particular L is a linear hydrocarbon chain having 1 to 4 chain members, more particularly L is a linear hydrocarbon chain having 1 to 2 chain members, even more particularly L is CH₂—CH₂;
V is CH or N, in particular V is N;
Z¹ is a linear hydrocarbon chain having 1 to 4 chain members, in particular Z¹ is a linear hydrocarbon chain having 1 to 2 chain members;
R¹ and R², R³ and R⁴ are, independently of one another, selected from H, C1-C6 alkyl, Na⁺ and K⁺, in particular R¹ and R² are, independently of one another, selected from H, C1-C4 alkyl and Na⁺, more particularly R¹ and R² are, independently of one another, selected from H, C1-C2 alkyl and Na⁺, more particularly R¹ and R² are, independently of one another, selected from H, methyl and Na⁺;
n is an integer of between 1 and 6, in particular n is an integer of between 1 and 5.

Other preferred dendrimers of formula (III) are those of formula (IIIB):

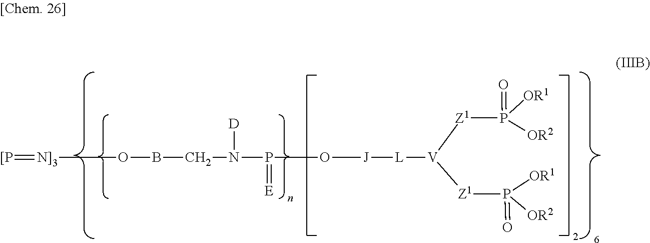

or the pharmaceutically acceptable salts thereof,
wherein
B, D, E, J, L, V, Z¹, R¹, R² and n are as defined above or in relation to formula (III),
{ }ₙ denotes the branching structure of the n generation chains of said dendrimer, and
[P═N]₃ denotes the central core of the following formula:

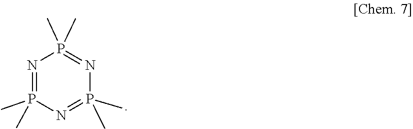

Preferred dendrimers of formula (IIIB) are those wherein s B, D, E, J,
L, V, Z¹, R¹, R² and n are defined as follows:
B is phenyl, optionally substituted by C1-C6 alkyl or halo, in particular B is phenyl;
D is selected from C1-C12 alkyl, CH₂—(CH₂—CH₂—O)ₐ—CH₃, (CH₂)_{b}—NHR′ and CH₂—(CH₂—CH₂—O)_{c}—CH₂—CH₂—CH₂—NHR′, where a, b and c are, independently of one another, integers of between 1 and 12 and R′ is H or

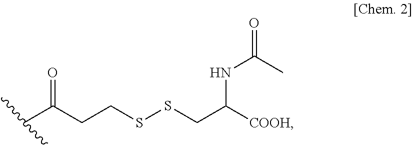 [SMILES: *C(=O)CCSSCC(NC(C)=O)C(=O)O]

in particular D is selected from C1-C10 alkyl, CH₂—(CH₂—CH₂—O)ₐ—CH₃, (CH₂)_{b}—NHR′ and CH₂—(CH₂—CH₂—O)_{c}—CH₂—CH₂—CH₂—NHR′, where a, b and c are, independently of one another, integers of between 1 and 10 and R′ is H or

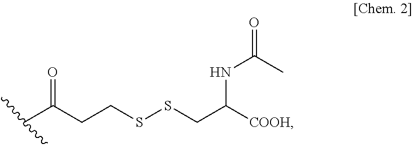 [SMILES: *C(=O)CCSSCC(NC(C)=O)C(=O)O]

more particularly D is selected from C1-C8 alkyl, CH₂—(CH₂—CH₂—O)ₐ—CH₃, (CH₂)_{b}—NHR′ and CH₂—(CH₂—CH₂—O)_{c}—CH₂—CH₂—CH₂—NHR′, where a, b and c are, independently of one another, integers of between 1 and 8 and R′ is H or

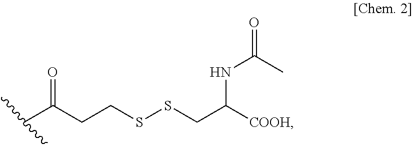 [SMILES: *C(=O)CCSSCC(NC(C)=O)C(=O)O]

more particularly still D is selected from C1-C8 alkyl, (CH₂)_{b}—NHR′ et CH₂—(CH₂—CH₂—O)_{c}—CH₂—CH₂—CH₂—NHR′, where b and c are, independently of one another, integers of between 1 and 6 and R′ is H or

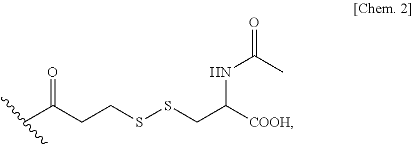 [SMILES: *C(=O)CCSSCC(NC(C)=O)C(=O)O]

even more particularly still D is selected from methyl, hexyl, octyl, (CH₂)₂ —NHR′ and CH₂—(CH₂—CH₂—O)₃—CH₂—CH₂—CH₂—NHR′, where R′ is

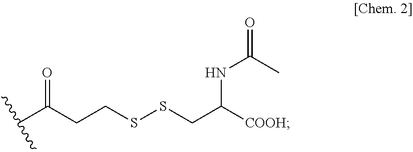 [SMILES: *C(=O)CCSSCC(NC(C)=O)C(=O)O]

E is O or S, in particular E is S;
J is phenyl, optionally substituted by C1-C6 alkyl or halo, in particular J is phenyl;
L is a linear or branched hydrocarbon chain having 1 to 4 chain members, in particular L is a linear hydrocarbon chain having 1 to 4 chain members, more particularly L is a linear hydrocarbon chain having 1 to 2 chain members, even more particularly L is CH₂—CH₂;
V is CH or N, in particular V is N;
Z¹ is a linear hydrocarbon chain having 1 to 4 chain members, in particular Z¹ is a linear hydrocarbon chain having 1 to 2 chain members;
R¹ and R² are, independently of one another, selected from H, C1-C6 alkyl, Na⁺ and K⁺, in particular R¹ and R² are, independently of one another, selected from H, C1-C4 alkyl and Na⁺, more particularly R¹ and R² are, independently of one another, selected from H, C1-C2 alkyl and Na⁺, more particularly R¹ and R² are, independently of one another, selected from H, methyl and Na⁺;
n is an integer of between 1 and 6, in particular n is an integer of between 1 and 5.

Other preferred dendrimers of formula (III) are those of formula (IIIC):

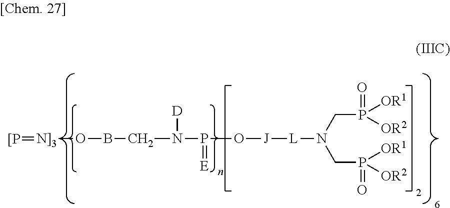

or the pharmaceutically acceptable salts thereof,
wherein
B, D, E, J, L, R¹, R² and n are as defined above or in relation to formula (III),
{ }ₙ denotes the branching structure of the n generation chains of said dendrimer, and
[P═N]₃ denotes the central core of the following formula:

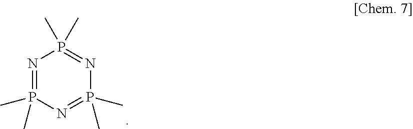

Preferred dendrimers of formula (IIIC) are those wherein B, D, E, J, L, R 1, R² and n are defined as follows:
B is phenyl, optionally substituted by C1-C6 alkyl or halo, in particular B is phenyl;
D is selected from C1-C12 alkyl, CH₂—(CH₂—CH₂—O)ₐ—CH₃, (CH₂)_{b}—NHR′ and CH₂—(CH₂—CH₂—O)_{c}—CH₂—CH₂—CH₂—NHR′, where a, b and c are, independently of one another, integers of between 1 and 12 and R′ is H or

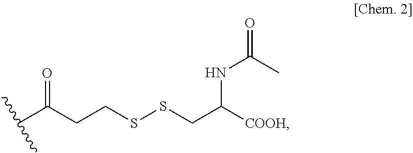 [SMILES: *C(=O)CCSSCC(NC(C)=O)C(=O)O]

in particular D is selected from C1-C10 alkyl, CH₂—(CH₂—CH₂—O)ₐ—CH₃, (CH₂)_{b}—NHR′ and CH₂—(CH₂—CH₂—O)_{c}—CH₂—CH₂—CH₂—NHR′, where a, b and c are, independently of one another, integers of between 1 and 10 and R′ is H or

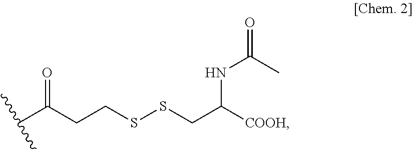 [SMILES: *C(=O)CCSSCC(NC(C)=O)C(=O)O]

more particularly D is selected from C1-C8 alkyl, CH₂—(CH₂—CH₂—O)ₐ—CH₃, (CH₂)_{b}—NHR′ and CH₂—(CH₂—CH₂—O)_{c}—CH₂—CH₂—CH₂—NHR′, where a, b and c are, independently of one another, integers of between 1 and 8 and R′ is H or

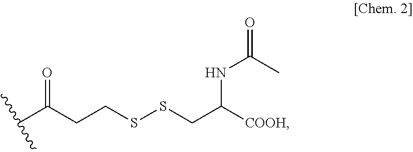 [SMILES: *C(=O)CCSSCC(NC(C)=O)C(=O)O]

more particularly still D is selected from C1-C8 alkyl, (CH₂)_{b}—NHR′ et CH₂—(CH₂—CH₂—O)_{c}—CH₂—CH₂—CH₂—NHR′, where b and c are, independently of one another, integers of between 1 and 6 and R′ is H or

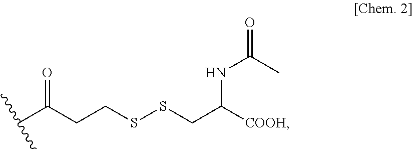 [SMILES: *C(=O)CCSSCC(NC(C)=O)C(=O)O]

even more particularly still D is selected from methyl, hexyl, octyl, (CH₂)₂ —NHR′ and CH₂—(CH₂—CH₂—O)₃—CH₂—CH₂—CH₂—NHR′, where R′ is

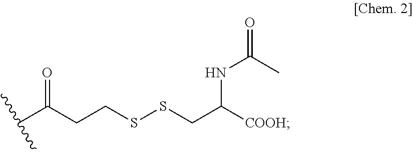 [SMILES: *C(=O)CCSSCC(NC(C)=O)C(=O)O]

E is O or S, in particular E is S;
J is phenyl, optionally substituted by C1-C6 alkyl or halo, in particular J is phenyl;
L is a linear or branched hydrocarbon chain having 1 to 4 chain members, in particular L is a linear hydrocarbon chain having 1 to 4 chain members, more particularly L is a linear hydrocarbon chain having 1 to 2 chain members, even more particularly L is CH₂—CH₂;
R¹ and R² are, independently of one another, selected from H, C1-C6 alkyl, Na⁺ and K⁺, in particular R¹ and R² are, independently of one another, selected from H, C1-C4 alkyl and Na⁺, more particularly R¹ and R² are, independently of one another, selected from H, C1-C2 alkyl and Na⁺, more particularly R¹ and
R² are, independently of one another, selected from H, methyl and Na⁺;
n is an integer of between 1 and 6, in particular n is an integer of between 1 and 5.

Other preferred dendrimers of formula (III) are those of formula (IIID):

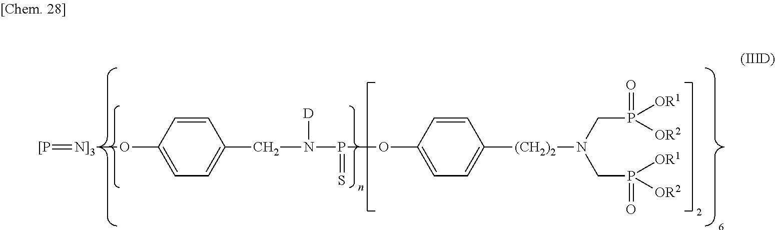

or the pharmaceutically acceptable salts thereof,
wherein
D, R¹, R² and n are as defined above or in relation to formula (III),
{ }ₙ denotes the branching structure of the n generation chains of said dendrimer, and
[P═N]₃ denotes the central core of the following formula:

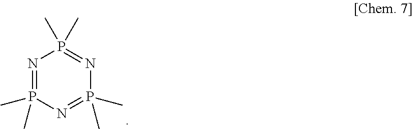

Preferred dendrimers of formula (IIID) are those wherein D, E, R¹, R² and n are defined as follows:
D is selected from C1-C12 alkyl, CH₂—(CH₂—CH₂—O)ₐ—CH₃, (CH₂)_{b}—NHR′ and CH₂—(CH₂—CH₂—O)_{c}—CH₂—CH₂—CH₂—NHR′, where a, b and c are, independently of one another, integers of between 1 and 12 and R′ is H or

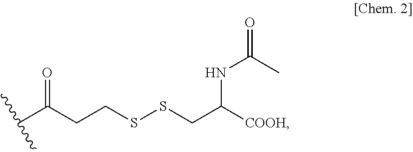 [SMILES: *C(=O)CCSSCC(NC(C)=O)C(=O)O]

in particular D is selected from C1-C10 alkyl, CH₂—(CH₂—CH₂—O)ₐ—CH₃, (CH₂)_{b}—NHR′ and CH₂—(CH₂—CH₂—O)_{c}—CH₂—CH₂—CH₂—NHR′, where a, b and c are, independently of one another, integers of between 1 and 10 and R′ is H or

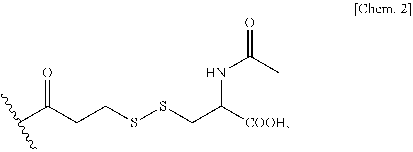 [SMILES: *C(=O)CCSSCC(NC(C)=O)C(=O)O]

more particularly D is selected from C1-C8 alkyl, CH₂—(CH₂—CH₂—O)ₐ—CH₃, (CH₂)_{b}—NHR′ and CH₂—(CH₂—CH₂—O)_{c}—CH₂—CH₂—CH₂—NHR′, where a, b and c are, independently of one another, integers of between 1 and 8 and R′ is H or

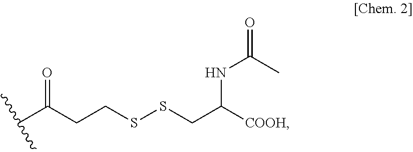 [SMILES: *C(=O)CCSSCC(NC(C)=O)C(=O)O]

more particularly still D is selected from C1-C8 alkyl, (CH₂)_{b}—NHR′ et CH₂—(CH₂—CH₂—O)_{c}—CH₂—CH₂—CH₂—NHR′, where b and c are, independently of one another, integers of between 1 and 6 and R′ is H or

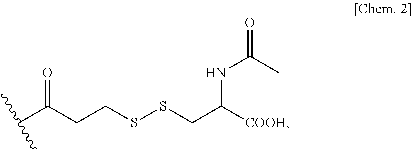 [SMILES: *C(=O)CCSSCC(NC(C)=O)C(=O)O]

even more particularly still D is selected from methyl, hexyl, octyl, (CH₂)₂ —NHR′ and CH₂—(CH₂—CH₂—O)₃—CH₂—CH₂—CH₂—NHR′, where R′ is

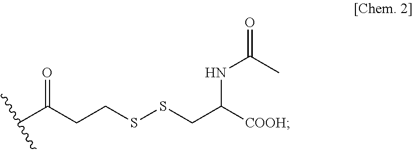 [SMILES: *C(=O)CCSSCC(NC(C)=O)C(=O)O]

R¹ and R² are, independently of one another, selected from H, C1-C6 alkyl, Na⁺ and K⁺, in particular R¹ and R² are, independently of one another, selected from H, C1-C4 alkyl and Na⁺, more particularly R¹ and R² are, independently of one another, selected from H, C1-C2 alkyl and Na⁺, more particularly R¹ and R² are, independently of one another, selected from H, methyl and Na⁺;
n is an integer of between 1 and 6, in particular n is an integer of between 1 and 5.

According to a particular embodiment, the generation chains of the dendrimer of the invention are identical.

According to another particular embodiment, the generation chains of the dendrimer of the invention are different. In particular, the generation chains of the dendrimer of the invention differ in the nature of the amine group at each generation. In other words, in formula (CG), D is different at each generation of the dendrimer. This is then referred to as a dendrimer of “layer-block” type.

According to this particular embodiment, the layer-block dendrimers can be represented by the following formula (IV):

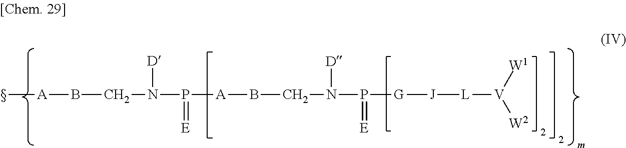

or the pharmaceutically acceptable salts thereof,
wherein
§, A, B, E, G, J, L, V, W¹, W², m are as defined previously or in formula (I), and
D′ and D″ are independently selected from C1-C12 alkyl, C1-C12 haloalkyl, aryl, heteroaryl, (CH₂—CH₂—O)ₐ—CH₃, CH₂—(CH₂—CH₂—O)ₐ—CH₃, (CH₂)_{b}—NR′R″ and CH₂—(CH₂—CH₂—O)_{c}—CH₂—CH₂—CH₂—NR′R″, where a, b and c are, independently of one another, integers of between 1 and 12, and R′ and R″ are, independently of one another, selected from H, C1-C12 alkyl, C1-C12 haloalkyl, tert-butyloxycarbonyl, fluorenylmethoxycarbonyl, methoxycarbonyl, tert-butylcarbonyl, p-toluenesulfonyl, methylsulfonyl, trifluoromethylsulfonyl, allyl, benzyl, trityl,

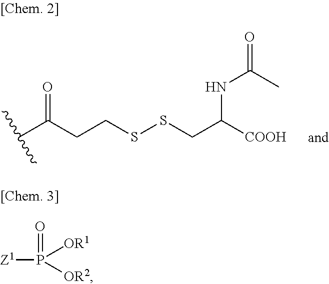

wherein Z¹ is a single bond or a straight or branched hydrocarbon chain having 1 to 6 chain members, one or more chain members optionally being a heteroatom, and R¹ and R² are independently selected from H, C1-C6-alkyl and M⁺, wherein M⁺ is a cation; in particular, D′ and D″ are independently selected from C1-C12-alkyl, C1-C12-haloalkyl, aryl, heteroaryl, (CH₂—CH₂—O)ₐ—CH₃ and CH₂—(CH₂—CH₂—O)ₐ—CH₃, where a is an integer from 1 to 12; more particularly, D′ and D″ are independently selected from C1-C12-alkyl, C1-C12-haloalkyl, (CH₂—CH₂—O)ₐ—CH₃ and CH₂—(CH₂—CH₂—O)ₐ—CH₃ where a is an integer from 1 to 12; still more particularly, D′ and D″ are independently selected from C1-C12-alkyl and (CH₂—CH₂—O)ₐ—CH₃ where a is an integer from 1 to 12; even more particularly, D′ and D″ are independently selected from C1-C12-alkyl and (CH₂—CH₂—O)ₐ—CH₃ where a is an integer from 1 to 6; even more particularly, D′ and D″ are independently selected from C1-C10-alkyl and CH₂—CH₂—O)ₐ—CH₃ where a is an integer between 1 and 4; even more particularly, D′ and D″ are independently selected from C1-C8-alkyl and (CH₂—CH₂—O)ₐ—CH₃ where a is an integer between 1 and 3; very particularly, D′ is C1-C8-alkyl and D″ is C1-C10-alkyl or (CH₂—CH₂—O)ₐ—CH₃ where a is an integer between 1 and 6; more particularly, D′ is C1-C4-alkyl and D″ is C1-C8-alkyl or (CH₂—CH₂—O)ₐ—CH₃ where a is an integer between 1 and 3; even more particularly, D′ is methyl and D″ is octyl or (CH₂—CH₂—O)₃—CH₃.

Particularly preferred compounds of the invention are those listed in table 1 below:

TABLE 1

| Com- | |
|---|---|
| pound | Structure |
| 1 | 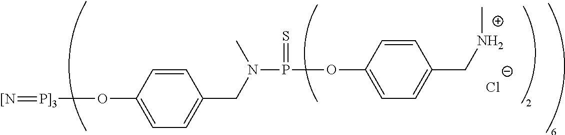 |
| 2 | 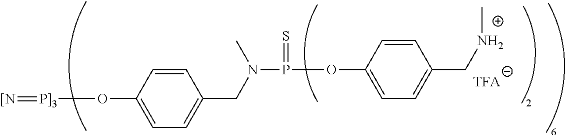 |
| 3 | 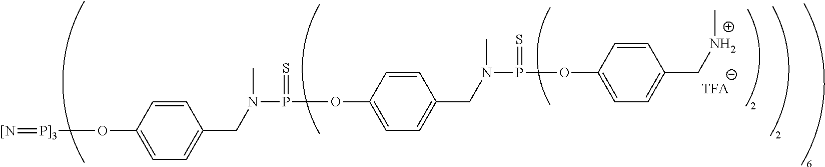 |
| 4 | 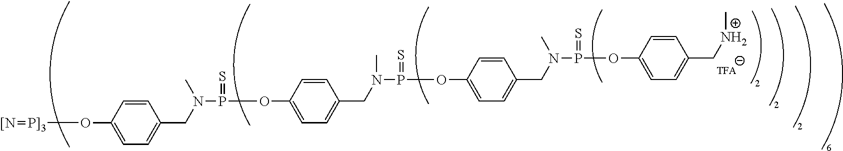 |
| 5 | 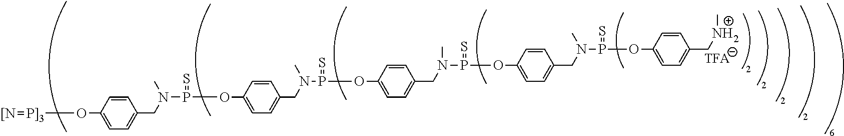 |
| 6 | 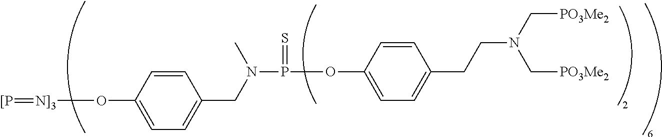 |
| 7 | 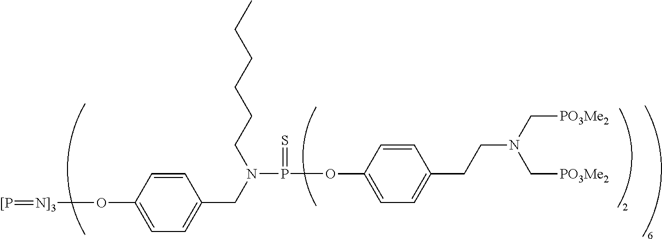 |
| 8 | 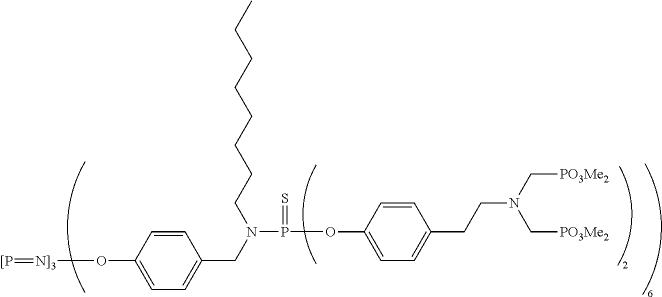 |
| 9 | 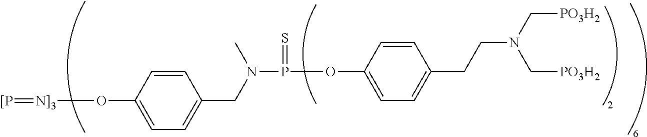 |
| 10 | 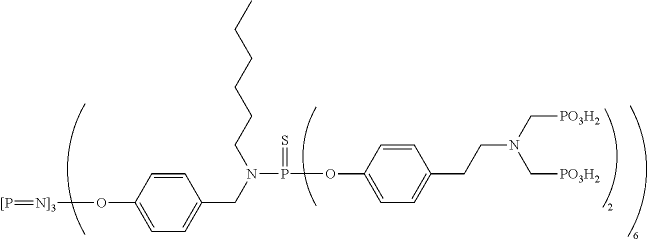 |
| 11 | 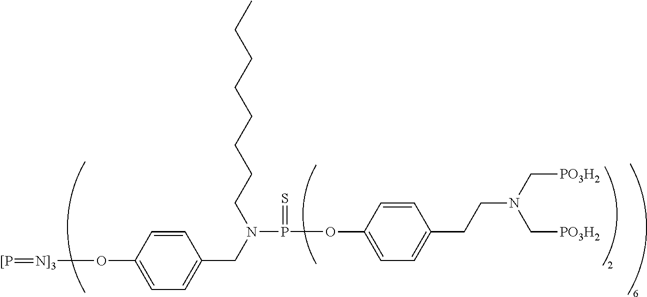 |
| 12 | 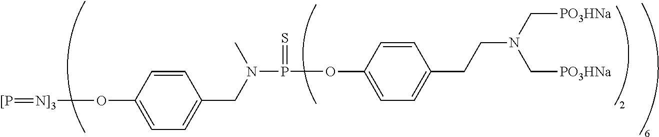 |
| 13 | 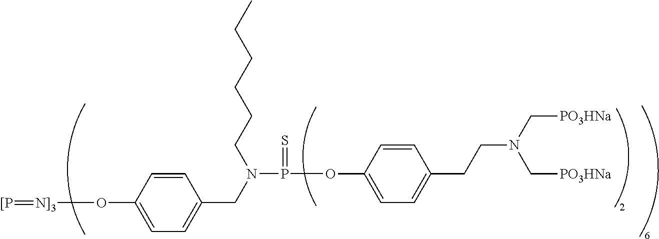 |
| 14 | 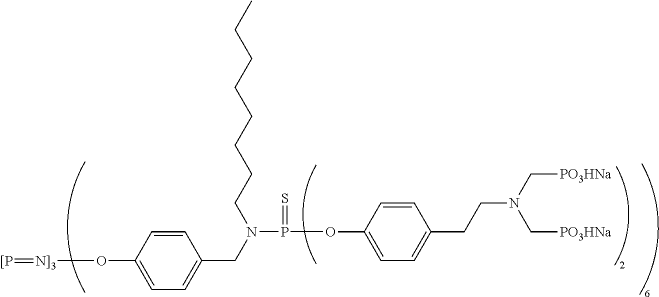 |
| 15 | 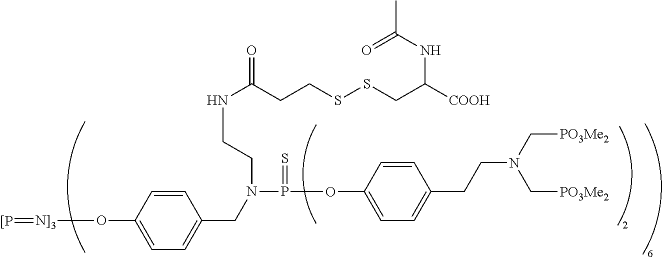 |
| 16 | 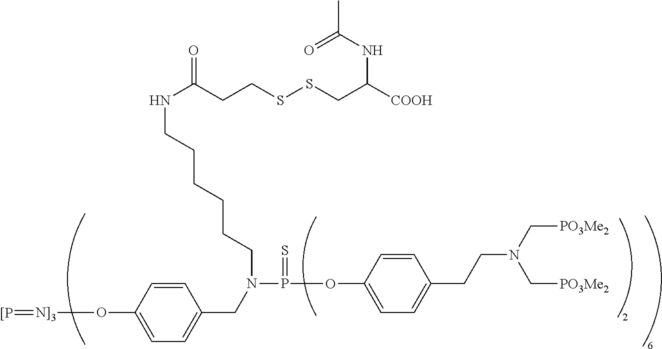 |
| 17 | 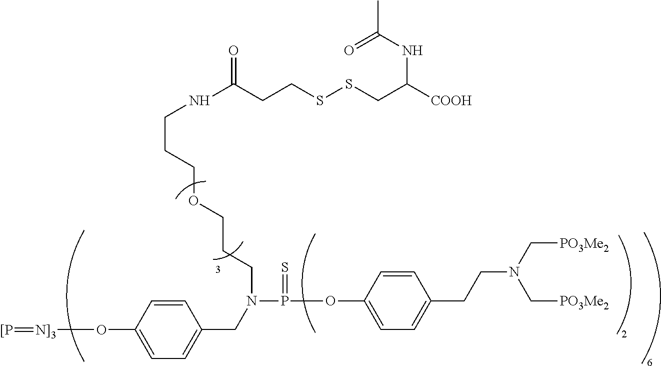 |
| 18 | 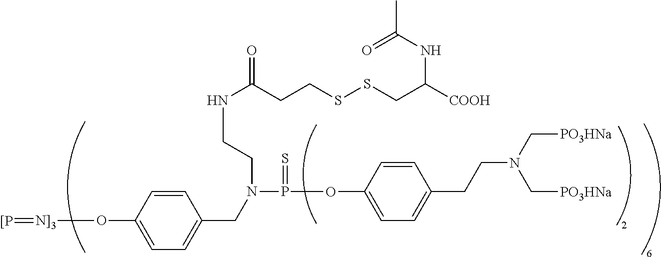 |
| 19 | 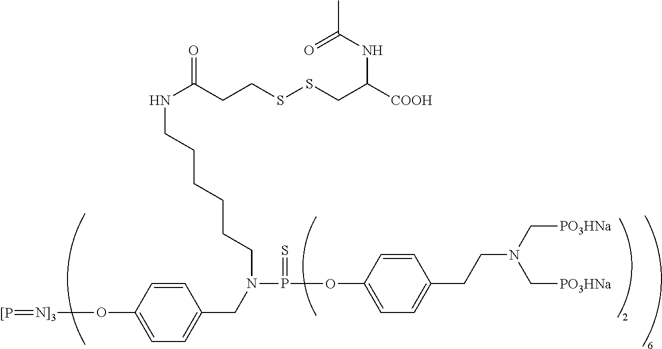 |
| 20 | 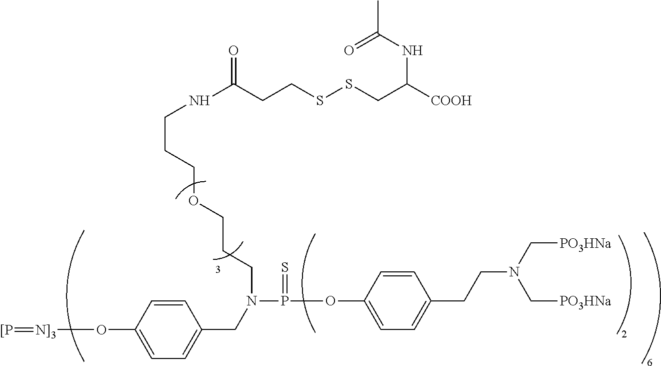 |
| 21 | 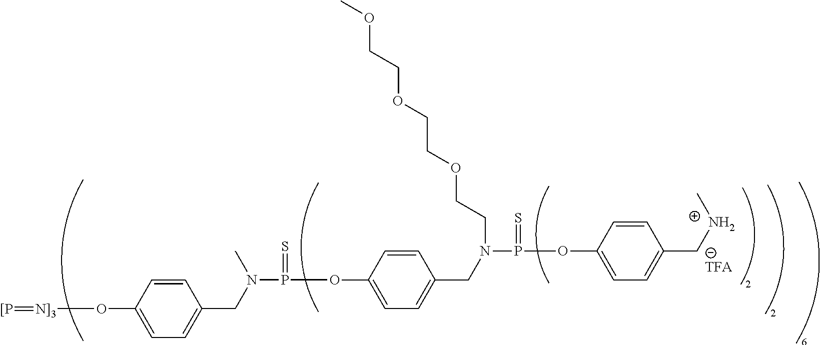 |
| 22 | 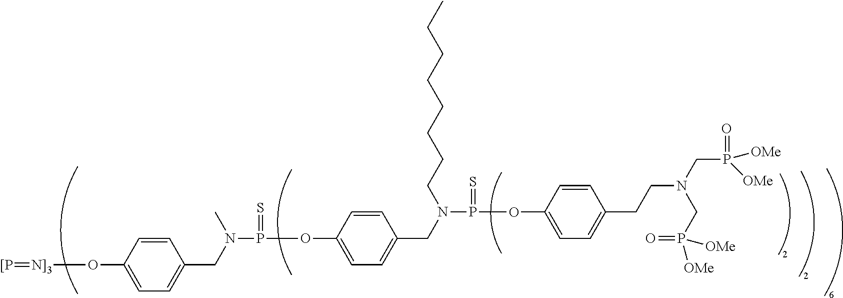 |

Advantageously, the dendrimers according to the invention exhibit improved stability at physiological pH compared to the phosphorus-containing dendrimers of the polyphosphorhydrazone type of the prior art. In particular, the dendrimers of the invention, especially the aminobismethylene phosphonic acid-terminated dendrimers (sodium monosalt), show no chemical degradation after a storage time of 4 to 8 months at a pH of 7.2, 7, 5 or 4 at a temperature of 25° C.

### Preparation Methods

Another subject matter of the present patent application is methods for preparing the dendrimer as described previously.

The dendrimer of the invention can be prepared by applying or adapting any method which is known per se and/or which is within the scope of the person skilled in the art.

The dendrimer of the invention can be prepared by the divergent route or by the semi-convergent route.

In a first alternative, the dendrimer is prepared by the divergent route.

Thus, according to this alternative, the dendrimer of the invention may be prepared by a method comprising the following steps, in succession:
(a) preparing an intermediate of the following formula:

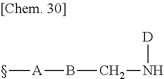

or a pharmaceutically acceptable salt thereof,
wherein
§, A, B and D are as defined previously or in formula (I);
(b) reacting the intermediate obtained in step (a) with P(E)X₃ where E is O or S, preferably E is S, and X is a halogen, preferably X is Cl;
(c) reacting the product obtained in step (b) with a fragment comprising an intermediate chain and optionally an end group.

In particular, step (a) is carried out by reacting the halogenated central core § with a generation chain precursor comprising an amine or ammonium function, or with a generation chain precursor comprising an aldehyde function followed by conversion of the aldehyde function into an amine or ammonium function.

In particular, the halogenated central core § used in step (a) is selected from the groups of the following formulae:

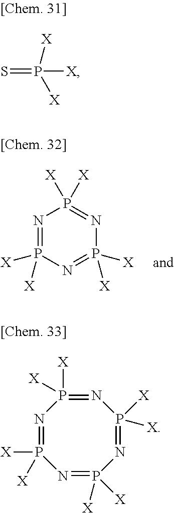

where X is a halogen, preferably X is Cl. More particularly, the halogenated central core § used in step (a) is

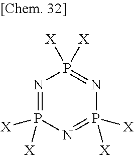

where X is a halogen, preferably X is Cl.

In one embodiment, the method further comprises, after step (a), a step of reacting the product obtained in step (a) with a generation chain precursor comprising an amine or ammonium function, or with a generation chain precursor comprising an aldehyde function followed by conversion of the aldehyde function into an amine or ammonium function, and repeating step (b).

In a second alternative, the dendrimer is prepared by the semi-convergent route.

Thus, according to this alternative, the dendrimer of the invention may be prepared by a method comprising the following steps, in succession:
(a) preparing a fragment comprising a generation chain, an intermediate chain and an end group of the following formula:

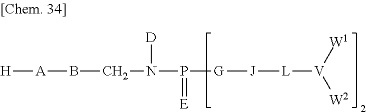

wherein
A, B, D, E, G, J, L, V, W¹ and W² are as defined previously or in formula (I);
(b) reacting the fragment obtained in step (a) with a halogenated central core §.

In particular, the halogenated central core § used in step (b) is selected from the groups of the following formulae:

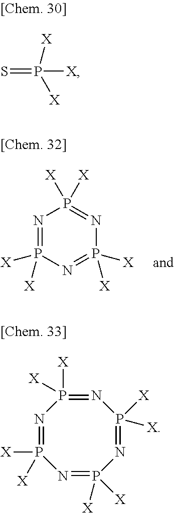

where X is a halogen, preferably X is Cl. More particularly, the halogenated central core § used in step (a) is

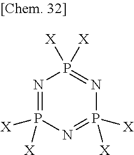

where X is a halogen, preferably X is Cl.

Advantageously, step (b) is carried out at a temperature of between −10000 and 5000, in particular between −78° C. and 3000.

Advantageously, step (b) is carried out in a solvent selected from apolar solvents and polar aprotic solvents. In particular, step (b) is carried out in a solvent selected from ether-type solvents and chlorinated solvents. More particularly, step (b) is carried out in a solvent selected from tetrahydrodfuran, diethyl ether, dioxane, dichloromethane and chloroform. More particularly still, step (b) is carried out in tetrahydrofuran.

Pharmaceutical Composition

Another subject matter of the present application is a pharmaceutical composition comprising the dendrimer as described previously, in particular the dendrimer of formula (III) or the sub-formulae thereof, and a pharmaceutically acceptable excipient.

The term “pharmaceutically acceptable” refers solely to the ingredients of a pharmaceutical composition which are compatible with one another and are nor harmful to the patient. In one embodiment, a pharmaceutically acceptable excipient does not produce any side effects, allergic reactions or other unwanted reactions when it is administered to an animal, preferably a human. For human administration, preparations must meet standard criteria for sterility, pyrogenicity, general safety, and purity as required by regulatory agencies, such as the FDA or EMA.

The pharmaceutically acceptable excipients can in particular be any excipient known to the person skilled in the art.

The pharmaceutical composition can also comprise, in addition to a dendrimer of the present invention as active ingredient, one or more additional therapeutic agents and/or active ingredients such as anti-inflammatories, antiinfective agents, or immunosuppressants.

Therapeutic Use

Another subject matter of the present patent application is the dendrimer of the invention, in particular the dendrimer of formula (III) or the sub-formulae thereof, or the pharmaceutical composition as described previously, for use thereof as medicament, more particularly for the treatment or prevention of an inflammatory disease in a subject in need thereof.

The present invention, according to another aspect thereof, relates to a method for the treatment or prevention of an inflammatory disease comprising administering, to a subject, a therapeutically effective dose of a dendrimer of the invention, in particular the dendrimer of formula (III) or the sub-formulae thereof, or of a pharmaceutical composition according to the invention.

Preferably, the subject is an animal, preferably a mammal, more preferentially a human suffering from an inflammatory disease.

The present invention also relates to the use of a dendrimer of the invention, in particular the dendrimer of formula (III) or the sub-formulae thereof, or of the pharmaceutical composition as described previously, for the manufacture of a medicament for the treatment or prevention of an inflammatory disease.

According to one embodiment, the inflammatory disease is selected from chronic inflammatory diseases, autoimmune inflammatory diseases and pro-inflammatory and inflammatory conditions associated with a cancer.

According to a particular embodiment, the chronic inflammatory diseases are selected from rheumatoid polyarthritis, psoriasis, uveitis and multiple sclerosis.

According to one embodiment, the dendrimer of the invention, in particular the dendrimer of formula (III) or the sub-formulae thereof, or the pharmaceutical composition of the invention, can be used as a medicament in particular for the treatment or prevention of an inflammatory disease as described previously in a subject in combination with at least one another therapeutic agent. Such additional therapeutic agents comprise, without being limited thereto, anti-inflammatories, antiinfective agents, or immunosuppressants. The dendrimer and the additional therapeutic agent can be administered at the same time or at different times, simultaneously or separately.

Thus, the treatment methods and the pharmaceutical compositions of the present invention can use the dendrimer of the invention, in particular the dendrimer of formula (III) or the sub-formulae thereof, or the pharmaceutical composition of the invention in the form of a monotherapy, but these methods and compositions can also be used in the form of a polytherapy wherein one or more dendrimers of the invention are co-administered in combination with one or more other therapeutic agents.

### Definitions

The definitions and explanations below relate to the terms and expressions used in the present application, including the description as well as the claims.

For the description of the compounds of the invention, the terms and expressions used must, unless indicated otherwise, be interpreted according to the below definitions.

The term “halo”, alone or as part of another group, denotes fluoro, chloro, bromo, or iodo. The preferred halo groups are chloro and fluoro, with fluoro being particularly preferred.

The term “alkyl”, alone or as part of another group, denotes a hydrocarbon radical of formula CₙH₂ₙ₊₁ wherein n is an integer greater than or equal to 1.

The term “haloalkyl”, alone or as part of another group, denotes an alkyl radical as defined above, wherein one or more hydrogen atoms are replaced by a halo group as defined above. The haloalkyl radicals according to the present invention may be linear or branched and comprise, without being limited thereto, radicals of formula CₙF₂ₙ₊₁ wherein n is an integer greater than or equal to 1, preferably an integer of between 1 and 10. Preferred haloalkyl radicals comprise trifluoromethyl, difluoromethyl, fluoromethyl, pentafluoroethyl, heptafluoro-n-propyl, nonafluoro-n-butyl, 1,1,1-trifluoro-n-butyl, 1,1,1-trifluoro-n-pentyl and 1,1,1-trifluoro-n-hexyl, trifluoromethyl.

The term “cycloalkyl”, alone or as part of another group, denotes a saturated monocyclic, bicyclic or tricyclic hydrocarbon radical having 3 to 12 carbon atoms, particularly 5 to 10 carbon atoms, more particularly 6 to 10 carbon atoms. Suitable cycloalkyl radicals comprise, without being limited thereto, cyclopropyl, cyclobutyl, cyclopentyl, cyclohexyl, cycloheptyl, cyclooctyl, norbornyl, adamantyl, particularly adamant-1-yl and adamant-2-yl, 1-decalinyl.

The term “aryl”, alone or as part of another group, denotes a polyunsaturated aromatic hydrocarbon having a single ring (phenyl) or several aromatic rings fused together (for example naphthyl), typically containing 5 to 12 atoms, preferably 6 to 10, wherein at least one of the rings is aromatic. A particularly preferred aryl group is phenyl.

The term “heteroaryl”, alone or as part of another group, denotes, without being limited thereto, aromatic rings or ring systems containing one to two rings fused together, typically containing 5 to 12 atoms, wherein at least one of the rings is aromatic, and wherein one or more carbon atoms in one or more of these rings are replaced with atoms of oxygen, nitrogen and/or sulfur, the nitrogen and sulfur heteroatoms optionally being oxidized and the nitrogen heteroatoms optionally being quaternized. Preferred, but non-limiting, heteroaryl groups are pyridinyl, pyrrolyl, furanyl, thiophenyl.

The compounds of the invention containing a basic functional group may be in the form of pharmaceutically acceptable salts. The pharmaceutically acceptable salts of the compounds of the invention containing one or more basic functional groups comprise in particular the acid addition salts thereof. Suitable acid addition salts are formed from acids which form non-toxic salts. Examples of salts comprise acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogenphosphate/dihydrogenphosphate, pyroglutamate, saccharate, stearate, succinate, tannate, tartrate, tosylate, trifluoroacetate and xinafoate. Particular examples of salts comprise hydrochloride/chloride and trifluoroacetate.

Pharmaceutically acceptable salts of compounds of the invention can for example be prepared as follows:
(i) react the compound of the invention with the desired acid, or
(ii) convert a salt of the compound of the invention into another by reacting with a suitable acid or by means of a suitable ion-exchange column.

All these reaction are generally carried out in solution. The salt can precipitate from the solution and be collected by filtration or can be recovered by evaporating the solvent. The degree of ionization in the salt may vary from completely ionized to virtually unionized.

The compounds of the invention can exist in the form of solvates, i.e. in the form of combinations with one or more molecules of solvent, for example ethanol or water. When the solvent is water, the term “hydrate” is used.

All the references to the compounds of the invention also denote the solvates thereof.

The compounds of the invention are the compounds of formula I and the solvates thereof as defined above, including all the polymorphs and crystalline forms thereof, the prodrugs thereof and the compounds or solvates having an isotopic label.

The term “subject” refers to an animal, more particularly a mammal. Preferably, the subject is a human being. For the purposes of the present invention, a subject may be a patient, i.e. a person receiving medical care, undergoing or having undergone a medical treatment, or being monitored in the context of the development of a disease.

The term “human” denotes subjects of both sexes and at all stages of development (that is, neonatal, infant, child, adolescent and adult). In one embodiment, this is an adolescent or an adult, preferably an adult.

The terms “treat” and “treatment” should be understood by their general meaning and thus comprise the improvement and resolution of a disease state.

The terms “prevent” and “prevention” denote the act of preventing or delaying the appearance of a disease or disorder and the associated symptoms, thus excluding a patient from developing a disease or disorder or reducing the risk of a patient developing a disease or disorder.

The term “therapeutically effective dose” or “effective dose” denotes the dose of active ingredient (dendrimer according to the invention) which is sufficient to achieve the desired therapeutic or prophylactic result in the patient to whom it is administered. In the present application, the therapeutically effective dose denotes the dose of dendrimer which is sufficient to have an anti-inflammatory action.

The present invention will be better understood with reference to the following examples. These examples represent certain embodiments of the invention and in no way limit the scope of the invention. The figures serve to illustrate the experimental results.

### BRIEF DESCRIPTION OF THE DRAWINGS

FIG. 1 shows the structure of the dendrimer 1-G′″₁-HCl (compound 1).

FIG. 2 shows the structure of the dendrimer 1-G′″₁-HTFA (compound 2).

FIG. 3 shows the structure of the dendrimer 1-G′″₂-HTFA (compound 3).

FIG. 4 shows the structure of the dendrimer 1-G′″₃-HTFA (compound 4).

FIG. 5 shows the structure of the dendrimer 1-G′″₄-HTFA (compound 5).

FIG. 6 shows the structure of the dendrimer 3-G′₁(OMe) (compound 6).

FIG. 7 shows the structure of the dendrimer 3-G′₁(OH) (compound 7).

FIG. 8 shows the structure of the dendrimer 3-G′₁(ONa) (compound 8).

FIG. 9 shows the structure of the dendrimer 4-G′₁(OMe) (compound 9).

FIG. 10 shows the structure of the dendrimer 4-G′₁(OH) (compound 10).

FIG. 11 shows the structure of the dendrimer 4-G′₁(ONa) (compound 11).

FIG. 12 shows the structure of the dendrimer 5-G′₁(OMe) (compound 12).

FIG. 13 shows the structure of the dendrimer 5-G′₁(OH) (compound 13).

FIG. 14 shows the structure of the dendrimer 5-G′₁(ONa) (compound 14).

FIG. 15 shows the structure of the dendrimer 11a-G′″₁(NH-NAC (compound 15).

FIG. 16 shows the structure of the dendrimer 11 b-G′″₁(NH-NAC (compound 16).

FIG. 17 shows the structure of the dendrimer 11c-G′″₁(NH-NAC (compound 17).

FIG. 18 shows the structure of the dendrimer 11a-G₁(NH-NAC/PO₃HNa) (compound 18).

FIG. 19 shows the structure of the dendrimer 11 b-G₁(NH-NAC/PO₃HNa) (compound 19).

FIG. 20 shows the structure of the dendrimer 11c-G₁(NH-NAC/PO₃HNa) (compound 20).

FIG. 21 shows the structure of the dendrimer 12-G′″₂-HTFA (compound 21).

FIG. 22 shows the structure of the dendrimer 14-G₂(OMe) (compound 22).

FIG. 23 shows the ³¹P-{¹H} NMR spectra of the dendrimer ABP according to the prior art at pH=7.2 at 25° C. A: t=0, B: t=4 days, C: t=10 days, D: t=60 days, E: t=8 months.

FIG. 24 shows the ³¹P-{¹H} NMR spectra of the compound 3-G′1(ONa) according to the invention at pH=7.2 at 25° C. A: t=0, B: t=15 days, C: t=45 days, D: t=6 days, E: t=8 months.

FIG. 25 shows the ³¹P-{¹H} NMR spectra of the dendrimer ABP according to the prior art at pH=5 at 25° C. A: t=0, B: t=5 days, C: t=10 days, D: t=30 days, E: t=90 days.

FIG. 26 shows the ³¹P-{¹H} NMR spectra of the compound 3-G′1(ONa) according to the invention at pH=5 at 25° C. A: t=0, B: t=5 days, C: t=15 days, D: t=90 days, E: t=240 days.

FIG. 27 shows the ³¹P-{¹H} NMR spectra of the compound 4-G′1(ONa) according to the invention at pH=7 at 25° C. A: t=0, B: t=5 days, C: t=15 days, D:t=90 days, E:t=120 days.

FIG. 28 shows the ³¹P-{¹H} NMR spectra of the compound 5-G′1(ONa) according to the invention at pH=7 at 25° C. A: t=0, B: t=5 days, C: t=15 days, D:t=90 days, E:t=120 days.

FIG. 29 shows the ³¹P-{¹H} NMR spectra of the compound 5-G′1(ONa) according to the invention at pH=4 at 25° C. A: t=0, B: t=5 days, C: t=15 days, D:t=90 days, E:t=120 days.

FIG. 30 shows flow cytometric analysis of the morphology of monocytes cultured with the dendrimer 3-G′₁(ONa).

### EXAMPLES

### Chemical Synthesis

All the temperatures are expressed in ° C. and all the reactions were carried out at ambient temperature (AT) unless indicated otherwise.

The reactions were monitored using thin-layer chromatography (TLC) carried out on ready-to-use sheets of aluminum covered with a silica gel and a fluorescence indicator UV254 (Kieselgel® 60 F254 Merck, 0.2 mm thick) or equivalent, or by ³¹P NMR.

Preparatory column chromatographies were carried out by the silica gel chromatography method, using silica with a particle size of 63 to 200 μm, by Sigma Aldrich.

The NMR analyses were carried out on a 300 MHz, 400 MHz or 600 Mhz Bruker spectrometer. The spectra are recorded in solution in deuterated chloroform (CDCl₃), in deuterated dichloromethane (CD₂Cl₂), in deuterated acetonitrile (CD₃ CN), in deuterated methanol (CD₃OD) or else in deuterated dimethyl sulfoxide (DMSO-d6). The chemical shifts (δ) are given in ppm followed, for the proton spectra, by the multiplicity, where s, brs, d, t, q, dd, td, and m denote, respectively, singlets, broad singlets, doublets, triplets, quadruplets, doublets of doublets, triplets of doublets and multiplets (or incompletely resolved peaks). The multiplicities are followed, where appropriate, by the value of the coupling constants, denoted J and expressed in Hertz (Hz). All carbon (¹³C) and phosphorus (³¹P) NMR spectra were performed by removing the couplings with protons (¹³C-{¹H} NMR and ³¹P-{¹H}) NMR).

The solvents, reagents and starting materials were purchased from well-known chemicals suppliers such as Sigma Aldrich, Acros Organics, Fluorochem, Eurisotop, VWR International, Sopachem and Polymer. The solvents, unless indicated otherwise, were purified by distillation before use. The reagents and the starting materials, unless indicated otherwise, were used without additional purifications.

The following abbreviations were used:
DCM: dichloromethane,
DIPEA: diisopropylamine,
Eq. or equiv.: equivalent,
EtOAc: ethyl acetate,
EtOH: ethanol,
MeOH: methanol,
NMR: nuclear magnetic resonance,
AT: ambient temperature,
TFA: trifluoroacetic acid,
THF: tetrahydrofuran.

### Synthesis of Generation 1 to 5 Dendrimers

Scheme 1 describes the reaction scheme of a first generation dendrimer.

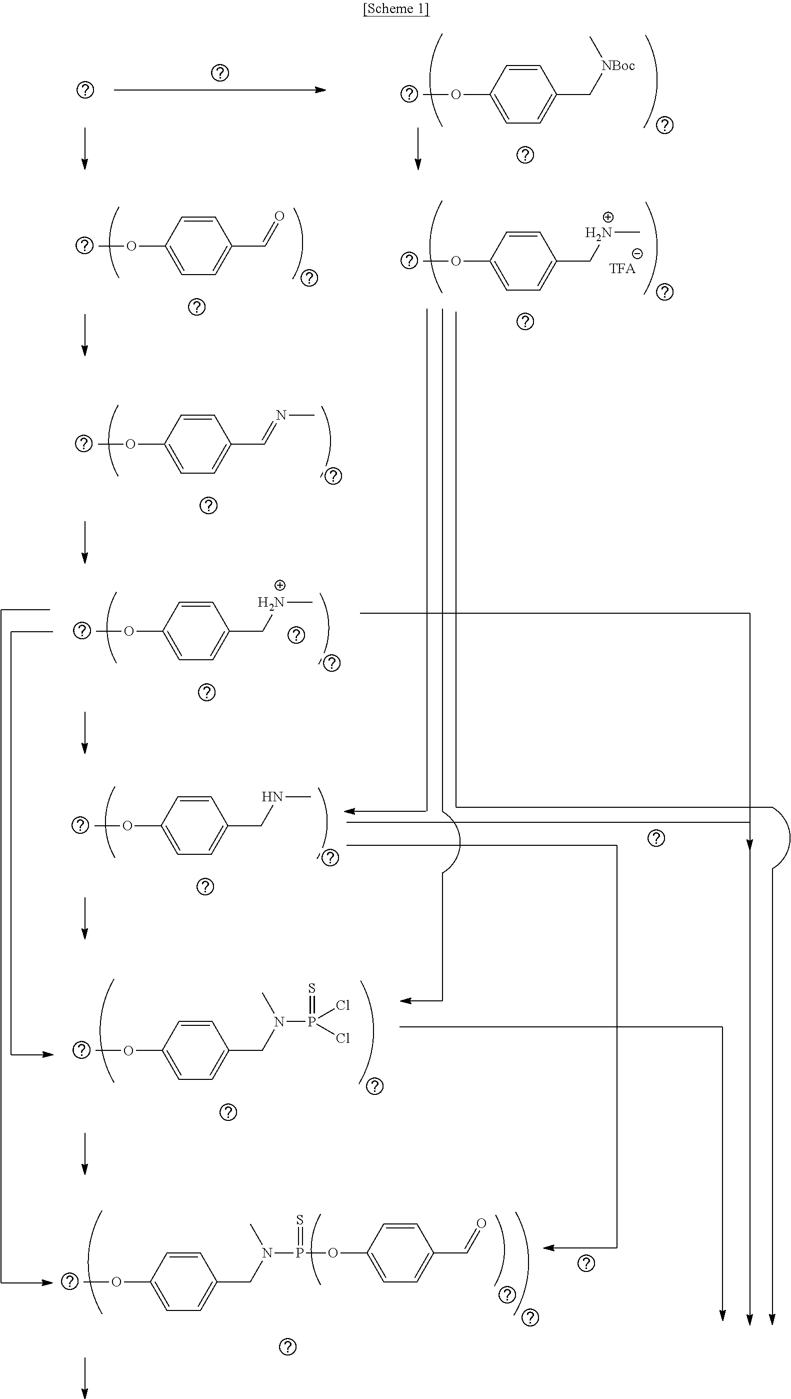

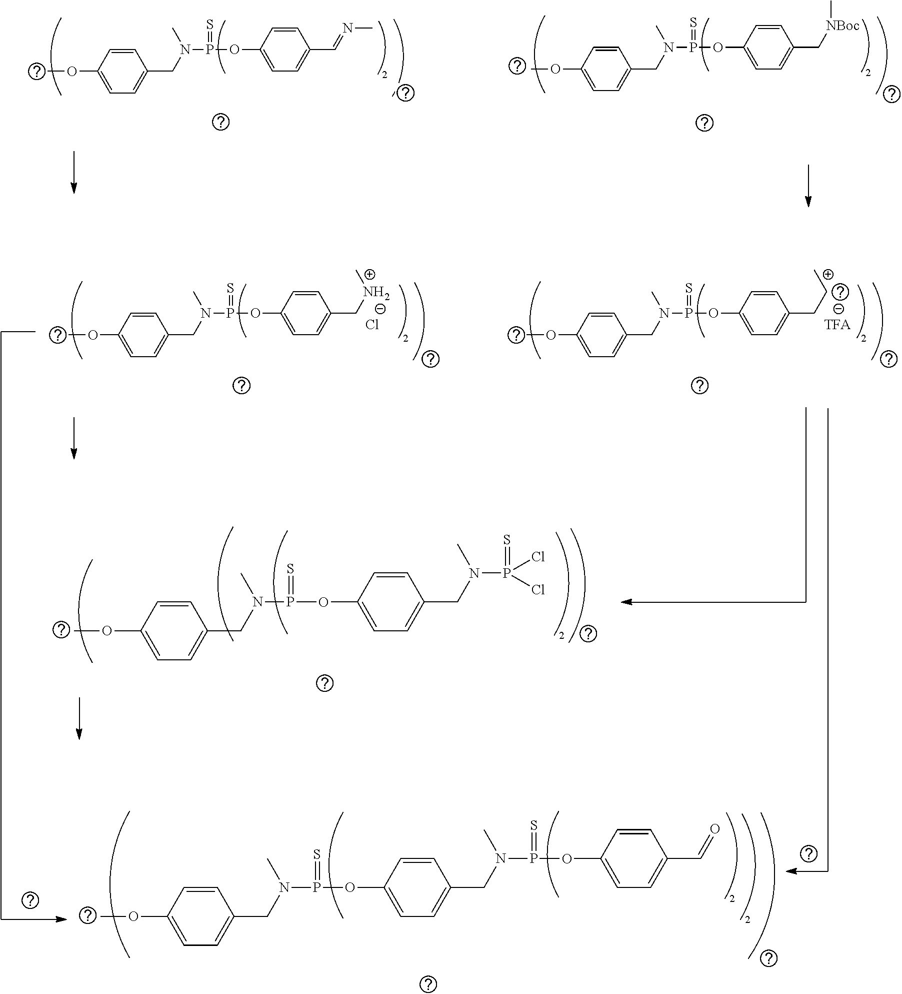

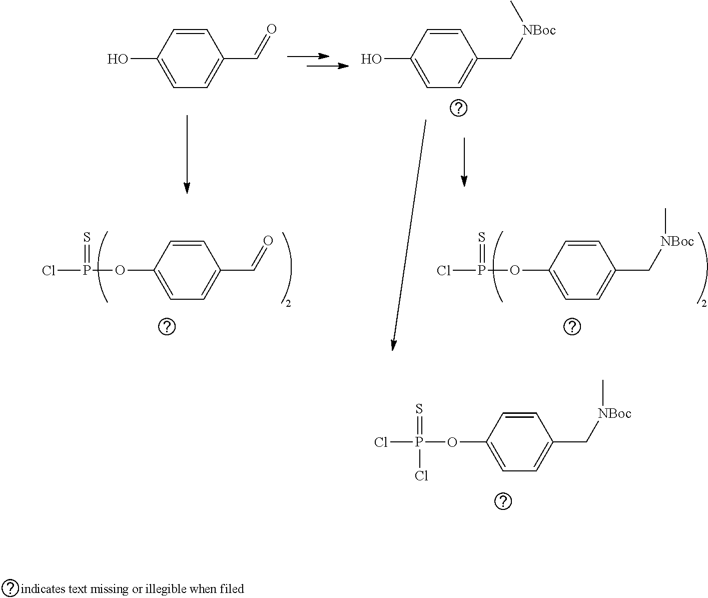

Numbering Used for the NMR Assignment:

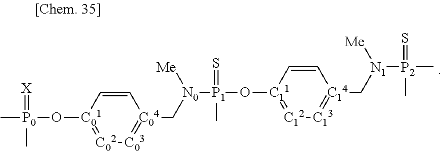

### Synthesis of Compound 1-G′₀ from N₃P₃Cl₆ (Macromolecules, 1981, 14, 1616-1622)

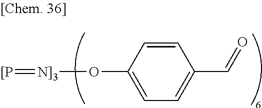

K₂CO₃ (14.3 g, 103.5 mmol) and 4-hydroxybenzaldehyde (4634 mg, 37.95 mmol) are added in succession to a solution of N₃P₃Cl₆ (2000 mg, 5.75 mmol) in THF (200 mL) at ambient temperature. After 72 h with stirring at ambient temperature, the mixture is filtered then concentrated to dryness. The crude residue is washed 4 to 5 times in MeOH to give compound 1-G′₀ in the form of a white powder at a yield of 81%.

¹H NMR (300 MHz, CDCl₃): δ (ppm) 9.83 (s, 6H, CHO), 7.74 (d, ³J_{HH}=8.6 Hz, 12H, C₀³H), 7.12 (d, ³J_{HH}=8.5 Hz, 12H, C₀²H).

³¹P-{¹H} NMR (121 MHz, CDCl³): δ (ppm) 7.15 (s, P₀).

¹³C-{¹H} NMR (75 MHz, CDCl₃): δ (ppm) 190.47 (s, CHO), 154.44 (dd, ²J_{CP}=5.1 Hz, ⁴J_{CP}=2.5 Hz, C₀¹), 133.74 (s, C₀⁴), 131.38 (s, C₀³), 121.21 (d, ³J_{CP}=2.5 Hz, C₀²).

### Synthesis of Compound 1-G″₀ from 1-G′₀

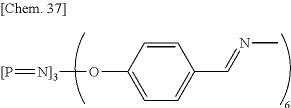

A solution of methylamine at 8 M in EtOH (1.3 mL, 10.45 mmol) is added to a solution of compound 1-G′₀ (500 mg, 0.580 mmol) in THF (50 mL) at ambient temperature. After stirring overnight at ambient temperature, the mixture is concentrated to dryness to give the dendrimer 1-G″₀ in the form of a white powder.

¹H NMR (300 MHz, CD₃CN): δ (ppm) 8.24 (d, ⁴J_{HH}=1.7 Hz, 6H, CH═N), 7.55 (d, ³J_{HH}=8.6 Hz, 12H, C₀³H), 6.99 (d, ³J_{HH}=8.5 Hz, 12H, C₀²H), 3.50 (s, 18H, N—CH₃)

³¹P-{¹H} NMR (121 MHz, CD₃CN): δ (ppm) 8.66 (s, P₀).

¹³C-{¹H}NMR (75 MHz, CD₃CN): δ (ppm) 160.80 (s, C═N), 151.57 (dd, ²J_{CP}=5.1 Hz, ⁴J_{CP}=2.5 Hz, C₀¹), 133.94 (s, C₀⁴) 129.13 (C₀³), 120.66 (dd, ³J_{CP}=3.2 Hz, ⁵J_{CP}=1.8 Hz, C₀²), 47.29 (s, CH₃).

### Synthesis of Compound 1-AB-Boc

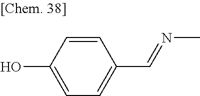

Methylamine (8 M in EtOH) (3.07 mL, 24.56 mmol) is added to a solution of 4-hydroxybenzaldehyde (1000 mg, 8.188 mmol) in THF (50 mL). After stirring overnight at ambient temperature, the reaction is terminated. Compound 1 is not isolated and is directly used thereafter.

¹H NMR (400 MHz, CD₃OD): δ (ppm) 8.16 (s, 1H, CH═N), 7.52 (d, J=8.7 Hz, 2H, C₀³H), 6.77 (d, J=8.6 Hz, 2H, C₀²H), 2.40 (s, 3H, CH₃).

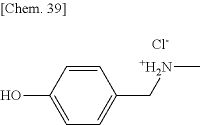

NaBH₄ (371 mg, 9.81 mmol) is subsequently added to the reaction medium. After 12 h of stirring at ambient temperature, the mixture is concentrated to dryness under reduced pressure. The crude residue is then dissolved in a solution of HCl (1 M in MeOH) (29.43 mL, 29.43 mmol). After 3 h of stirring at ambient temperature, the solution is filtered then concentrated under reduced pressure to give a white powder. The powder is washed with 3×50 mL of CH₂Cl₂ to give the corresponding amine hydrochloride in the form of a white powder at a yield of 98%.

¹H NMR (300 MHz, CD₃OD): δ (ppm) 7.35 (d, ³J_{HH}=8.7 Hz, 2H, C₀³H), 6.87 (d, ³J_{HH}=8.7 Hz, 2H, C₀²H), 4.10 (s, 2H, CH₂), 2.69 (s, 3H, CH₃).

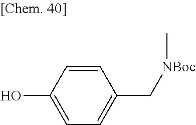 [SMILES: CN(Cc1ccc(O)cc1)C(=O)OC(C)(C)C]

Boc₂O (2386 mg, 10.93 mmol) is added to a solution of the previously obtained amine hydrochloride (2000 mg, 11.51 mmol) in CH₂Cl₂ (200 mL) in the presence of DIPEA (1.942 mL, 10.93 mmol). After 72 h at ambient temperature, the mixture is washed directly 1 time with 100 mL of distilled water at pH=5. The organic phase is recovered, dried with Na₂SO₄, filtered and concentrated under vacuum. The powder obtained is subsequently washed with 3×50 mL of hexane to give the compound 1-AB-Boc in the form of a white powder at a yield of 75%.

¹H NMR (300 MHz, CDCl₃): δ (ppm) 7.07 (d, ³J_{HH}=8.5 Hz, 2H, C₀³H), 6.83 (d, ³J_{HH}=7.6 Hz, 2H, C₀²H), 4.35 (s, 2H, CH₂), 2.81 (s, 3H, CH₃), 1.51 (s, 9H, Boc).

¹³C-{¹H} NMR (75 MHz, CDCl₃: δ (ppm) 156.30 (s, NC(O)OtButyl), 155.68 (s, C₀¹), 129.12 (s, C₀⁴), 128.73 (s, C₀³), 115.51 (s, C₀²), 80.21 (s, O—C-tButyl), 52.11 (CH₂ NBoc), 51.32 (CH₂NBoc), 33.78 (s, N—CH₃), 28.51 (s, C(CH₃)₃).

### Synthesis of Compound 1-AB₂-CHO (Mitjaville J. et al., J. Am. Chem. Soc., 1994, 116, 5007-5008)

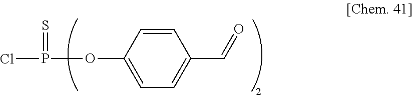

A solution of 4-hydroxybenzaldehyde (2000 mg, 16.38 mmol) with Et₃N (4.566 mL, 32.76 mmol) in THF (50 mL) is added dropwise over a duration of 30 min to a solution of PSCl₃ (0.810 mL, 7.99 mmol), in THF (200 mL) with molecular sieve, cooled to −78° C. After addition, the mixture slowly rises to ambient temperature. After 12 h of stirring at ambient temperature, the mixture is concentrated to dryness under reduced pressure. The crude residue is subsequently purified using silica gel chromatography with a 5/95→10/90 EtOAc/Hexane gradient, to give compound 1-AB₂-CHO in the form of an orange oil at a yield of 76%.

³¹P-{¹H} NMR (121 MHz, CDCl₃): δ (ppm) 55.86 (s, P₀).

### Synthesis of Compound 1-AB₂-Boc

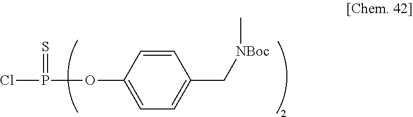

A solution of compound AB-Boc (2000 mg, 3.50 mmol) with Et₃N (1.025 mL, 7.36 mmol) in CH₂Cl₂ (30 to 50 mL) is added dropwise over a duration of 30 min to a solution of PSCl₃ (0.173 mL, 1.70 mmol) in CH₂Cl₂ (200 mL), with molecular sieve, cooled to −78° C. After addition, the reaction mixture rises slowly to ambient temperature. After 72 h of stirring at ambient temperature, the mixture is concentrated to dryness under reduced pressure. The crude residue is subsequently purified using silica gel chromatography with a 5/95→10/90 EtOAc/Hexane gradient, to give compound 1-AB₂-Boc in the form of a transparent oil at a yield of 84%.

¹H NMR (400 MHz, CDCl₃): δ (ppm) 7.28 (s, 4H, CH-arom), 4.44 (s, 2H, CH₂), 2.85 (s, 3H, CH₃), 1.49 (s, 9H, Boc).

³¹P-{¹H} NMR (162 MHz, CDCl₃): δ (ppm) 58.82 (s, P₀).

¹³C-{¹H} NMR (75 MHz, CDCl₃): δ (ppm) 155.85 (brs, NC(O)OtButyl), 149.26 (d, ²J_{CP}=10.0 Hz, C₀¹), 136.53 (s, C₀⁴), 128.73 (brs, C₀³), 121.30 (d, ³J_{CP}=5.2 Hz, C₀²), 79.87 (s, O—C-tButyl), 52.00 (brs, CH₂NBoc), 51.28 (brs, CH₂NBoc), 34.07 (s, N—CH₃), 28.43 (s, C (CH₃)₃).

### Synthesis of Compound 1-G′″₀-Boc from N₃P₃Cl₆

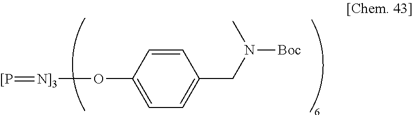

Cs₂CO₃ (6.30 mmol) and compound 1-AB-Boc (3.15 mmol) are added in succession to a solution of hexachlorocyclotrisphophazene (0.5 mmol) in THF (50 mL). After stirring overnight at ambient temperature, the mixture is centrifuged, filtered then concentrated under reduced pressure. The crude residue is subsequently purified using silica gel chromatography with a CH₂Cl₂/EtOAc) mixture to give dendrimer 1-G′″₀-Boc in the form of a white powder at a yield of 86%.

³¹P-{¹H} NMR (162 MHz, CDCl₃): δ (ppm) 8.2 (s, P₀).

### Synthesis of Compound 1-G′″₀-HCl from 1-G″₀

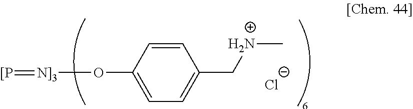

NaBH₄ (201 mg, 5.32 mmol) is added to a solution of dendrimer 1-G″₀ (500 mg, 0.53 mmol) in a THF/MeOH (25/5) mL mixture. After stirring overnight at ambient temperature, the mixture is concentrated to dryness. The crude residue is diluted in 200 mL of DCM then washed once with 50 mL distilled water. The organic phase is recovered, dried with Na₂SO₄, filtered and concentrated under reduced pressure. The product is then dissolved in MeOH (30 mL) to which 9.5 mL of HCl (1 M in MeOH) is added. After 2 h of stirring at ambient temperature, the solution is filtered then concentrated under reduced pressure. The residue is washed 3 times with 25 mL CH₂Cl₂ to give the product 1-G′″₀-HCl (protonated HCl form) in the form of a white powder.

¹H NMR (400 MHz, CD₃OD): δ (ppm) 7.54 (d, ³J_{HH}=8.5 Hz, 12H, C₀³H), 7.05 (d, ³J_{HH}=8.3 Hz, 12H, C₀²H), 4.27 (s, 12H, CH₂NH), 2.75 (s, 18H, N—CH₃).

³¹P-{¹H} NMR (162 MHz, CD₃OD): δ (ppm) 8.29 (s, P₀).

¹³C-{¹H} NMR (101 MHz, CD₃OD): δ (ppm) 151.07 (dd, ²J_{CP}=5.1 Hz, ⁴J_{CP}=2.5 Hz, C₀¹), 131.46 (s, C₀³), 128.71 (s, C₀⁴), 121.12 (dd, ³J=3.2, ⁵J=1.7 Hz, (s, C₀²), 51.30 (s, CH₂NH), 31.88 (s, N—CH₃).

### Synthesis of Compound 1-G′″₀-HTFA from 1-G′″₀-Boc

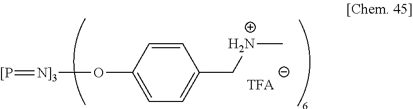

Compound 1-G′″₀-HTFA (protonated TFA form) is obtained by dissolving compound 1-G′″₀-Boc (300 mg) in 10 mL of a CH₂Cl₂/TFA (50/50) mixture for 60 minutes at ambient temperature then evaporating the reaction mixture to dryness.

³¹P-{¹H} NMR (162 MHz, CD₃OD): δ (ppm) 8.3 (s, P₀).

### Synthesis of Compound 1-G′″₀ from 1-G′″₁-HTFA or 1-G′″₁-HCl

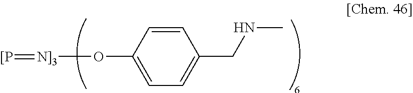

Compound 1-G′″₀-HTFA or 1-G′″₀-HCl (300 mg) is dissolved in 25 mL of distilled water to which 25 mL of a solution of NaOH (2M) is added. The aqueous phase is subsequently extracted 3 times with 200 mL of CH₂Cl₂. The organic phase is subsequently dried with Na₂SO₄, filtered and concentrated under vacuum to give dendrimer 1-G′″₀ in the form of a transparent oil at a yield of 80%.

¹H NMR (400 MHz, CDCl₃): δ (ppm) 7.12 (d, ³J_{HH}=8.5 Hz, 12H, C₀³H), 6.91 (d, ³J_{HH}=8.3 Hz, 12H, C₀²H), 3.69 (s, 12H, CH₂NH), 2.43 (s, 18H, N—CH₃).

³¹P-{¹H} NMR (162 MHz, CDCl₃): δ (ppm) 8.71 (s, P₀).

¹³C-{¹H} NMR (101 MHz, CDCl₃): δ (ppm) 149.55 (dd, ²J_{CP}=5.1 Hz, ⁴J_{CP}=2.5 Hz, C₀¹), 136.70 (s, C₀³), 128.99 (s, C₀⁴), 120.82 (dd, ³J=3.4, ⁵J=1.7 Hz, (s, C₀²), 55.43 (s, CH₂NH), 36.06 (s, N—CH₃).

### Synthesis of Compound 1-G₁ from 1-G′″₀

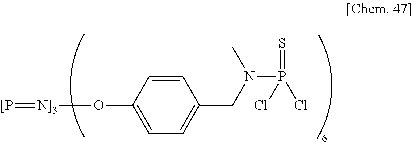

A solution of dendrimer 1-G′″₀ (800 mg, 0.85 mmol) is added dropwise to a solution of PSCl₃ (3.109 mL, 30.60 mmol) in CH₂Cl₂ (100 mL) in the presence of Et₃N (0.740 mL, 5.31 mmol) in CH₂Cl₂ (25 mL) over a duration of 30 min. After 3 h of stirring at ambient temperature, the mixture is concentrated to dryness under reduced pressure. The crude residue is subsequently dissolved in 200 mL of CH₂Cl₂ then filtered over silica gel to give dendrimer 1-G1 in the form of a white powder at a yield of 82%.

¹H NMR (300 MHz, CDCl₃): δ (ppm) 7.22 (d, ³J_{HH}=8.5 Hz, 12H, C₀³H), 6.99 (d, ³J_{HH}=8.3 Hz, 12H, C₀²H), 4.60 (d, ³J_{HP}=15.1 Hz, 12H, CH₂NH), 2.82 (d, ³J_{HP}=16.2 Hz, 18H, N—CH₃).

³¹P-{¹H} NMR (121 MHz, CDCl₃): δ (ppm) 64.12 (s, P₁, 8.35 (s, P₀).

¹³C-{¹H} NMR (75 MHz, CDCl₃): δ (ppm) 150.26 (dd, ²J_{CP}=5.1 Hz, ⁴J_{CP}=2.5 Hz, C₀¹), 132.73 (d, ³J_{CP}=6.5 Hz, C₀⁴), 129.17 (s, C₀³), 121.29-121.15 (m, C₀²), 54.18 (d, ²J_{CP}=5.5 Hz, CH₂NH), 35.11 (d, ²J_{CP}=2.5 Hz, N—CH₃).

### Synthesis of Compound 1-G₁ from 1-G′″₀-HTFA or 1-G′″₀-HCl

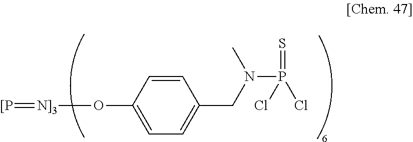

PSCl₃ (5 to 10 equivalents per terminal protonated amine, i.e. 5*0.05*2ⁿy to 10*0.05*2ⁿy mmol) is added very quickly to a solution of compound 1-G′″₀-HTFA or 1-G′″ₙ-HCl (0.05 mmol) in CH₂Cl₂ (25 mL) with a base, preferentially organic and non-nucleophilic, such as DIPEA (2 to 3 equivalents per terminal protonated amine, i.e. 2*0.05*2ⁿy to 3*0.05*2ⁿy mmol) cooled to −78° C. The mixture is slowly brought to ambient temperature. After stirring overnight at ambient temperature, the reaction mixture is evaporated to dryness under reduced pressure. The crude residue is diluted with 200 mL of CH₂Cl₂ and filtered over silica gel. The solution is subsequently dried over MgSO₄ and concentrated under reduced pressure. The residue obtained is washed 3 times with a CHCl₃/alkane ( 1/10) mixture to give the product G₁ in the form of a white powder.

### Synthesis of Compound 1-G₁ from 1-G₁

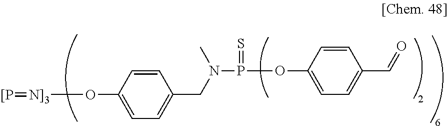

Cs₂CO₃ (1341 mg, 4.11 mmol) and 4-hydroxybenzaldehyde (314.2 mg, 2.57 mmol) are added in succession to a solution of dendrimer 1-G₁ (300 mg, 0.171 mmol) in THF (30 mL). After stirring overnight at ambient temperature, the mixture is centrifuged, filtered then concentrated under reduced pressure. The product is subsequently purified using silica gel chromatography (50/50 to 60/40 EtOAc/Hexane gradient), to give dendrimer 1-G′1 in the form of a white powder at a yield of 73%.

¹H NMR (400 MHz, CDCl₃): δ (ppm) 9.96 (s, 12H, CHO), 7.88 (d, ³J_{HH}=8.5 Hz, 24H, C₁³H), 7.33 (d, ³J_{HH}=8.3 Hz, 24H, C₁²H), 7.18 (d, ³J_{HH}=8.5 Hz, 12H, C₀³H), 6.97 (d, ³J_{HH}=8.5 Hz, 12H, C₀²H), 4.49 (d, ³J_{HP}=13.4 Hz, 12H, CH₂NH), 2.83 (d, ³J_{HP}=10.6 Hz, 18H, N—CH₃).

³¹P-{¹H} NMR (162 MHz, CDCl₃): δ (ppm) 66.34 (s, P₁), 8.35 (s, P₀).

¹³C-{¹H} NMR (101 MHz, CDCl₃): δ (ppm) 190.65 (s, CHO), 155.31 (d, ²J_{CP}=7.3 Hz, C₁¹), 150.14 (dd, ²J_{CP}=5.1 Hz, ⁴J_{CP}=2.5 Hz, C₀¹), 133.66 (d, ³J_{CP}=4.4 Hz, C₀⁴), 133.57 (d, ⁵J_{CP}=1.3 Hz, C₁⁴), 131.48 (s, C₁³), 129.42 (s, C₀³), 121.63 (d, ³J_{CP}=5.0 Hz, C₁²), 121.01 (m, C₀²), 53.67 (d, ²J_{CP}=7.8 Hz, CH₂NH), 33.72 (d, ²J_{CP}=1.5 Hz, N—CH₃).

### Synthesis of Compound 1-G′₁ from 1-G′″₀

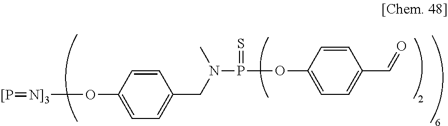

Compound 1-AB₂-CHO (0.63 mmol) is added to a solution of compound 1-G′″₀ (0.1 mmol) in THF (30 mL) in the presence of DIPEA (1.5 mmol). After stirring overnight at ambient temperature, the mixture is evaporated to dryness under reduced pressure. The crude residue is subsequently purified by silica gel chromatography to give compound 1-G′₁ in the form of a white powder.

### Synthesis of Compound 1-G₁ from 1-G′″₀-HTFA or 1-G′″₀-HCl

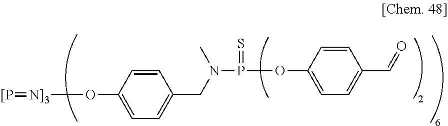

Compound 1-AB₂-CHO (0.63 mmol) is added to a solution of compound 1-G′″₀-TFA or 1-G′″₀-HCl (0.1 mmol) in THF (30 mL) in the presence of DIPEA (1.8 mmol). After stirring overnight at ambient temperature, the mixture is evaporated to dryness under reduced pressure. The crude residue is subsequently purified by silica gel chromatography to give compound 1-G′₁ in the form of a white powder.

### Synthesis of Compound 1-G″₁ from 1-G′₁

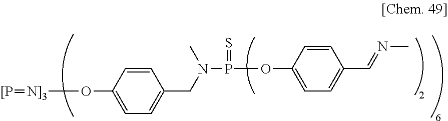

An 8M solution of methylamine in EtOH (320 μL, 2.59 mmol) is added to a solution of compound 1-G′₁ (200 mg, 0.072 mmol) in THF (20 mL). After stirring overnight at ambient temperature, the mixture is concentrated to dryness under reduced pressure to give the dendrimer 1-G″₁ in the form of a white powder. The yield is quantitative.

¹H NMR (300 MHz, CD₂Cl₁₂): δ (ppm) 8.25 (d, ⁴J_{HH}=1.6 Hz, 12H, CH═N), 7.71 (d, ³J_{HH}=8.5 Hz, 24H, C₁³H), 7.32-7.10 (m, 36H, C₁²H, C₀³H), 6.96 (d, ³J_{HH}=8.4 Hz, 12H, C₀²H), 4.48 (d, ³J_{HP}=13.0 Hz, 12H, CH₂N₀H), 3.48 (d, ⁴J_{HH}=1.5 Hz, 36H, C═N₁—CH₃), 2.80 (d, ³J_{HP}=10.7 Hz, 18H, N₀—CH₃).

³¹P-{¹H} NMR (121 MHz, CD₂Cl₁₂): δ (ppm) 67.60 (s, P¹), 8.59 (s, P₀).

### Synthesis of Dendrimer 1-G′″₁-HCl from 1-G″₁

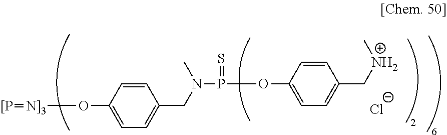

A 1 M solution of BH₃·THF in THF (792 μl, 0.792 mmol) is added to a solution of compound 1-G″1 (155 mg, 0.053 mmol) in THF (20 mL). After stirring overnight at ambient temperature, 4 mL of MeOH are added. After stirring for one hour, the reaction mixture is evaporated to dryness under reduced pressure. The crude residue is subsequently dissolved in a 1 M solution of HCl in MeOH (1908 μl, 1.908 mmol). The solution is subsequently filtered then concentrated to dryness under reduced pressure. The powder obtained is washed 3 times with 10 mL of CH₂Cl₂ to give compound 1-G′″₁-HCl (protonated form) in the form of a white powder at a yield of 90%.

¹H NMR (300 MHz, CD₃OD): δ (ppm) 7.59 (d, ³J_{HH}=8.5 Hz, 24H, C₁³H), 7.28 (m, 36H, C₁²H, C₀³H), 6.92 (d, ³J_{HH}=8.4 Hz, 12H, C₀²H), 4.51 (d, ³J_{HP}=14.0 Hz, 12H, CH₂N₀H), 4.21 (s, 24H, CH₂N₁H), 2.82 (d, ³J_{HP}=10.5 Hz, 18H, N₀—CH₃), 2.73 (s, 36H, N₁—CH₃).

³¹P-{¹H} NMR (121 MHz, CD₃OD): δ (ppm) 67.97 (s, P₁), 9.07 (s, P₀).

¹³C NMR (75 MHz, CD₃OD): δ (ppm) 151.81 (d, ²J_{CP}=7.4 Hz, C₁¹), 149.79 (dd, ²J_{CP}=4.4 Hz, ⁴J_{CP}=3.0 Hz, C₀¹), 134.33 (d, ³J_{CP}=4.5 Hz, C₀⁴), 131.35 (s, C₁³), 129.26 (s, C₀³), 128.33 (d, ⁵J_{CP}=1.2 Hz, C₁⁴), 121.61 (d, ³J_{CP}=4.5 Hz, C₁²), 120.65 (s, C₀²), 53.01 (d, ²J_{CP}=6.9 Hz, CH₂N₀H), 51.46 (s, CH₂N₁H), 33.00 (d, ²J_{CP}=1.6 Hz, N₀—CH₃), 31.85 (s, N₁—CH₃).

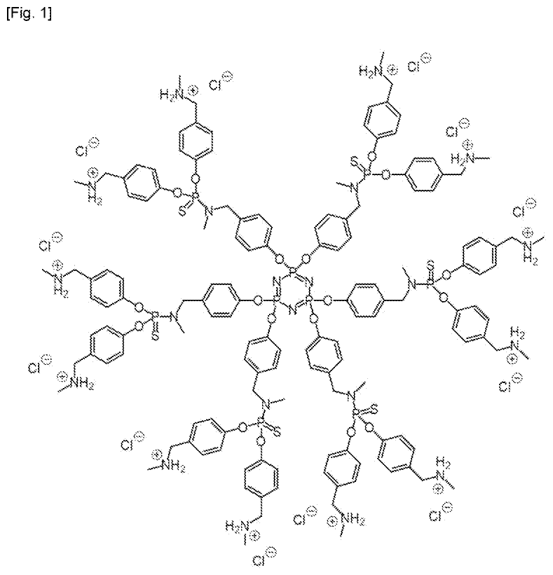

The structure of dendrimer 1-G′″₁-HCl is represented in [FIG. 1].

### Synthesis of Compound 1-G′″₁-Boc from 1-G₁

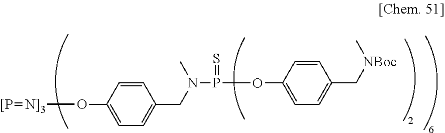

Cs₂CO₃ (5570 mg, 17.14 mmol) and compound 1-AB-Boc (2035 mg, 8.57 mmol) are added in succession to a solution of compound 1-G₁ (1000 mg, 0.571 mmol) in THF (50 mL). After stirring overnight at ambient temperature, the mixture is centrifuged, filtered then concentrated under reduced pressure. The crude residue is subsequently purified using silica gel chromatography with a CH₂Cl₂/EtOAc) mixture to give dendrimer 1-G′″₁-Boc in the form of a white powder at a yield of 76%.

¹H NMR (300 MHz, CDCl₃): δ (ppm) 7.26-7.11 (m, 60H, C₁³H, C₁²H, C₀³H), 6.94 (d, ³J_{HH}=8.4 Hz, 12H, C₀²H), 4.47 (d, ³J_{HP}=12.6 Hz, 12H, CH₂N₀H), 4.38 (s, 24H, CH₂N₁H), 2.87-2.71 (m, 54H, N₀—CH₃, N₁—CH₃), 1.47 (s, 108H, Boc).

³¹P-{¹H} NMR (121 MHz, CDCl₃): δ (ppm) 68.11 (s, P₁, 8.46 (s, P₀).

¹³C-{¹H} NMR (75 MHz, CDCl₃): δ (ppm) 156.07 (s, CO), 155.66 (s, CO), 150.22-149.91 (m, C₁¹, C₀¹), 135.02 (s, C₁⁴), 134.06 (d, ³J_{CP}=4.4 Hz, C₀⁴), 129.30 (s, C₀³), 128.63 (brs, C₁³), 121.09 (d, ³J_{CP}=4.8 Hz, C₁²), 120.95 (s, C₀²), 79.72 (s, O—C-tButyl), 53.56 (d, ²J_{CP}=7.1 Hz, CH₂N₀H), 51.64 (d, J=56.7 Hz, CH₂N₁Boc), 33.93 (s, N₁—CH₃), 33.65 (d, ²J_{CP}=1.7 Hz, N₀—CH₃), 28.45 (s, C(CH₃)₃).

### Synthesis of Compound 1-G′″₁-Boc from 1-G′″₀-HTFA or 1-G′″₀.-HCl

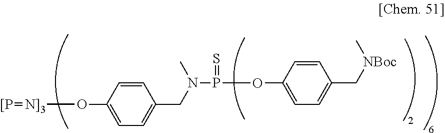

Compound 1-AB₂-Boc (1.26 mmol) is added to a solution of compound 1-G′″₁ (0.1 mmol) in THF (30 mL) in the presence of DIPEA (3 mmol). After stirring overnight at ambient temperature, the mixture is evaporated to dryness under reduced pressure. The crude residue is subsequently purified on a silica gel column to give compound 1-G′″₁ in the form of a white powder.

### Synthesis of Compound 1-G′″₁-Boc from 1-G′″₀

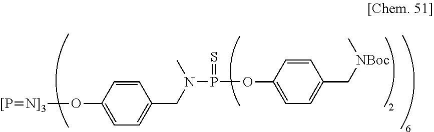

Compound 1-AB₂-Boc (1.26 mmol) is added to a solution of compound 1-G′″₁-TFA or 1-G′″₁-HCl (0.1 mmol) in THF (30 mL) in the presence of DIPEA (3.6 mmol). After stirring overnight at ambient temperature, the mixture is evaporated to dryness under reduced pressure. The crude residue is subsequently purified on a silica gel column to give compound 1-G′″₁ in the form of a white powder.

### Synthesis of Dendrimer 1-G′″₁-HTFA from 1-G′″₁-Boc

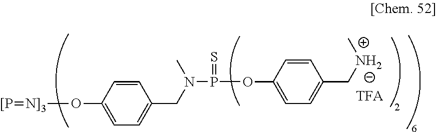

Compound 1-G′″ₙBoc (200 mg) is dissolved in a CH₂Cl₂/trifluoroacetic acid 70/30 mixture (30 mL). After 30 min of stirring at ambient temperature, the mixture is concentrated to dryness under reduced pressure. The residue is subsequently co-evaporated 5 times with 3 mL of CH₂Cl₂ to give dendrimer 1-G′″ₙ-HTFA in the form of a white powder.

¹H NMR (400 MHz, CD₃CN) δ (ppm): 2.64 (s), 2.77 (d, J=10.8 Hz), 4.11 (s), 4.47 (d, J=13.4 Hz), 6.93 (d, J=8.4 Hz), 7.23 (d, J=9.7 Hz), 7.47 (d, J=8.6 Hz).

³¹P-{¹H} NMR (162 MHz, CD₃CN) δ (ppm): 9.09 (s, P0), 67.80 (s, P1).

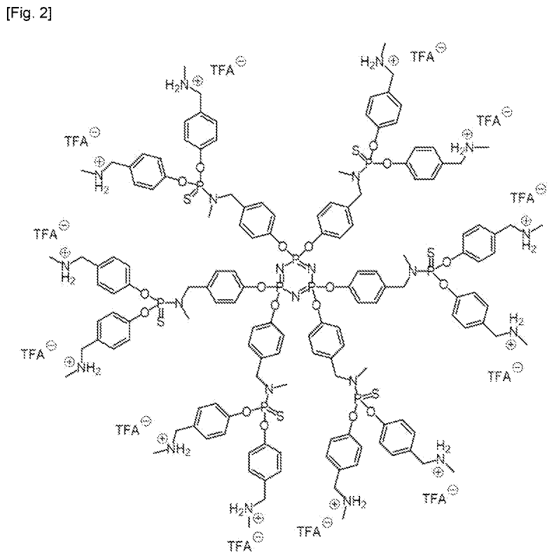

The structure of dendrimer 1-G′″₁-HTFA is represented in [FIG. 2].

### Synthesis of Compound 1-G₂ from 1-G′″₁-HTFA or 1-G′″₁-HCl

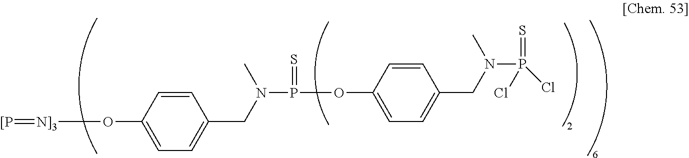

PSCl₃ (337 μl, 3.32 mmol) is added very quickly to a solution of 1-G′″₁-HTFA (200 mg, 0.046 mmol) or 1-G′″₁-HCl in CH₂Cl₂ (25 mL) with DIPEA (246 μl, 1.386 mmol) cooled to −78° C. After addition, the mixture slowly rises to ambient temperature. After stirring overnight at ambient temperature, the mixture is evaporated to dryness under reduced pressure. The crude residue obtained is diluted with 200 mL of CH₂Cl₂ and filtered over silica gel. The solution is subsequently dried with MgSO₄ and concentrated under reduced pressure. The residue obtained is subsequently washed 3 times with a CHCl₃/Pentane ( 1/10) mixture to give the product 1-G₂ in the form of a white powder. The yield is 73%.

¹H NMR (500 MHz, CDCl₃): δ (ppm) 7.33 (d, ³J_{HH}=8.5 Hz, 24H, C₁³H), 7.26-7.11 (m, 36H, C₀³H, C₁² H), 6.93 (d, ³J_{HH}=8.4 Hz, 12H), 4.60 (d, ³J_{HP}=14.9 Hz, 24H, CH₂N₁H), 4.50 (d, ³J_{HP}=12.8 Hz, 12H, CH₂N₀H), 2.92-2.77 (m, 54H, N₁—CH₃, N₀—CH₃).

³¹P-{¹H} NMR (202 MHz, CDCl₃): δ (ppm) 67.86 (s, P₁), 63.95 (s, P₂), 8.49 (s, P₀).

¹³C-{¹H} NMR (126 MHz, CDCl₃): δ (ppm) 150.68 (d, ²J_{CP}=7.5 Hz, C₁¹), 150.02 (dd, ²J_{CP}=5.2 Hz, ⁴J_{CP}=2.5 Hz, C₀¹), 133.99 (d, ³J_{CP}=4.6 Hz, C₀⁴), 132.73 (d, ³J_{CP}=6.6 Hz, C₁⁴), 129.33 (s, C₁³, C₀³), 121.41 (d, ³J_{CP}=4.8 Hz, s, C₁²), 120.97 (s, C₀²), 54.21 (d, ²J_{CP}=5.3 Hz, CH₂N₁H), 53.64 (d, ²J_{CP}=7.4 Hz, CH₂N₀H), 35.15 (s, N₁—CH₃), 33.74 (d, N₀—CH₃).

### Synthesis of Compound 1-G′₂ from 1-G′″₁-HTFA or 1-G′″₁-HCl

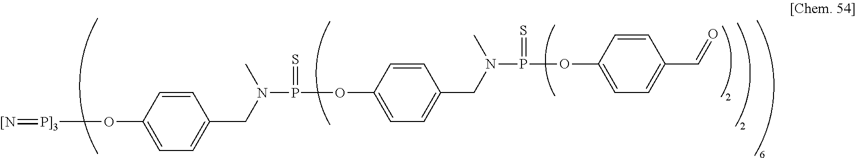

Compound 1-AB₂-CHO (1.26 mmol) is added to a solution of compound 1-G′″₁-HTFA or 1-G′″₁-HCl (0.1 mmol) in THF (30 mL) in the presence of DIPEA (3.6 mmol). After stirring overnight at ambient temperature, the mixture is evaporated to dryness under reduced pressure. The crude residue is subsequently purified on a silica gel column to give compound 1-G′₂ in the form of a white powder.

¹H NMR (400 MHz, CDCl₃): δ (ppm) 9.93 (s, 12H), 9.94 (s, 12H), 7.87 (dd, J=8.5, 2.1 Hz, 48H), 7.34 (d, J=8.5 Hz, 48H), 7.26 (s, 24H), 7.19 (d, J=8.5 Hz, 12H), 7.11 (d, J=8.3 Hz, 24H), 6.95 (d, J=8.4 Hz, 12H), 4.54-4.44 (m, 36H), 2.86 (d, J=10.8 Hz, 36H), 2.78 (d, J=10.6 Hz, 18H).

³¹P-{¹H} NMR (162 MHz, CDCl₃): δ (ppm) 67.91 (s, P₁), 66.33 (s, P₂), 8.43 (s, P₀).

### Synthesis of Compound 1-G₂ from 1-G₂

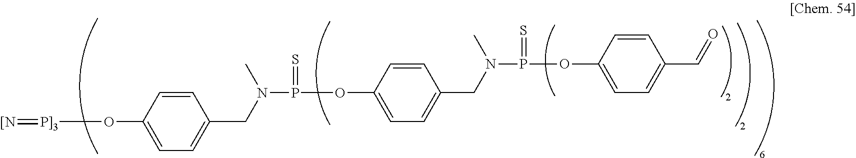

Cs₂CO₃ (7.2 mmol) and 4-hydroxybenzaldehyde (2.5 mmol) are added in succession to a solution of dendrimer 1-G₂ (0.15 mmol) in THF (30 mL). After stirring overnight at ambient temperature, the mixture is centrifuged, filtered then concentrated under reduced pressure. The product is subsequently purified using a silica gel chromatography column (50/50 to 60/40 EtOAc/Hexane gradient), to give dendrimer 1-G′₂ in the form of a white powder at a yield of 73%.

Scheme 2 describes the reaction scheme of generations 2 to 5 dendrimers from a 1st generation dendrimer.

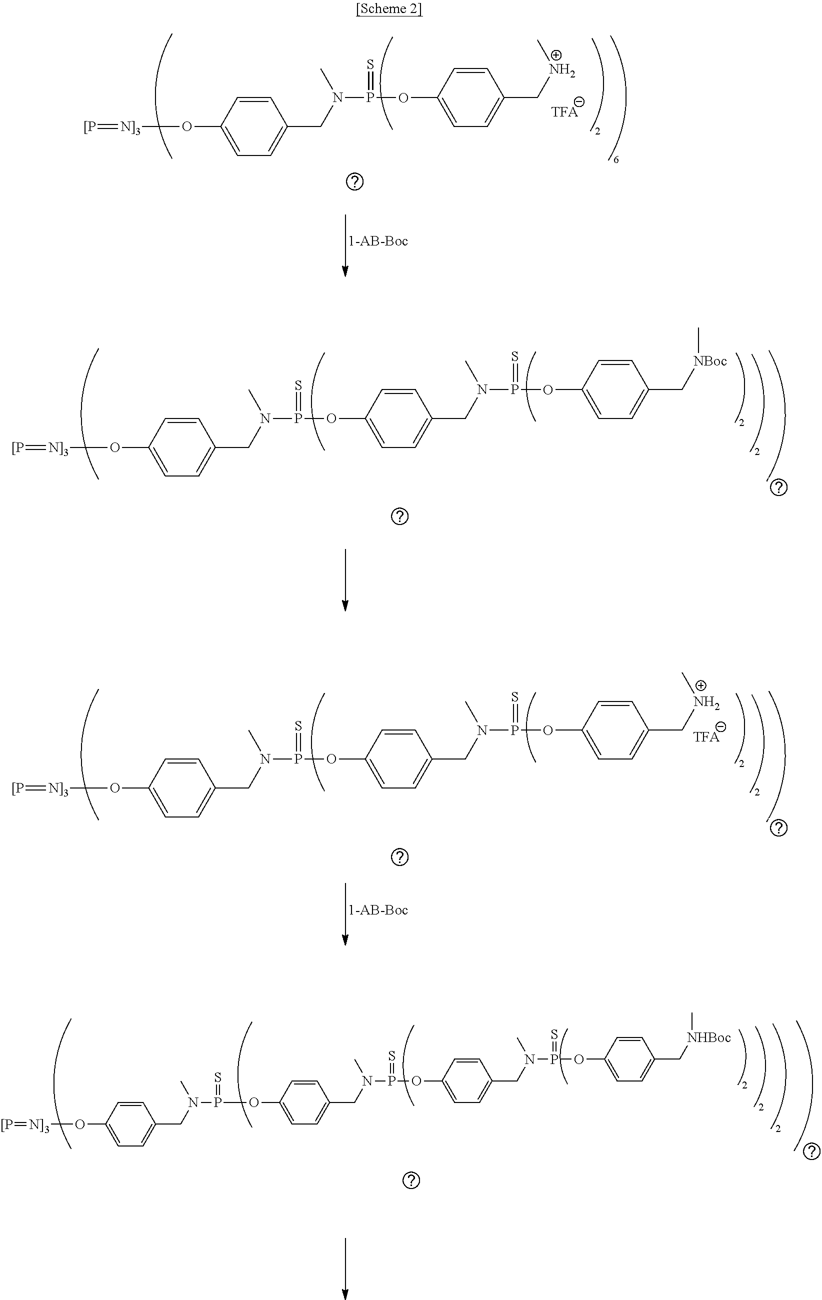

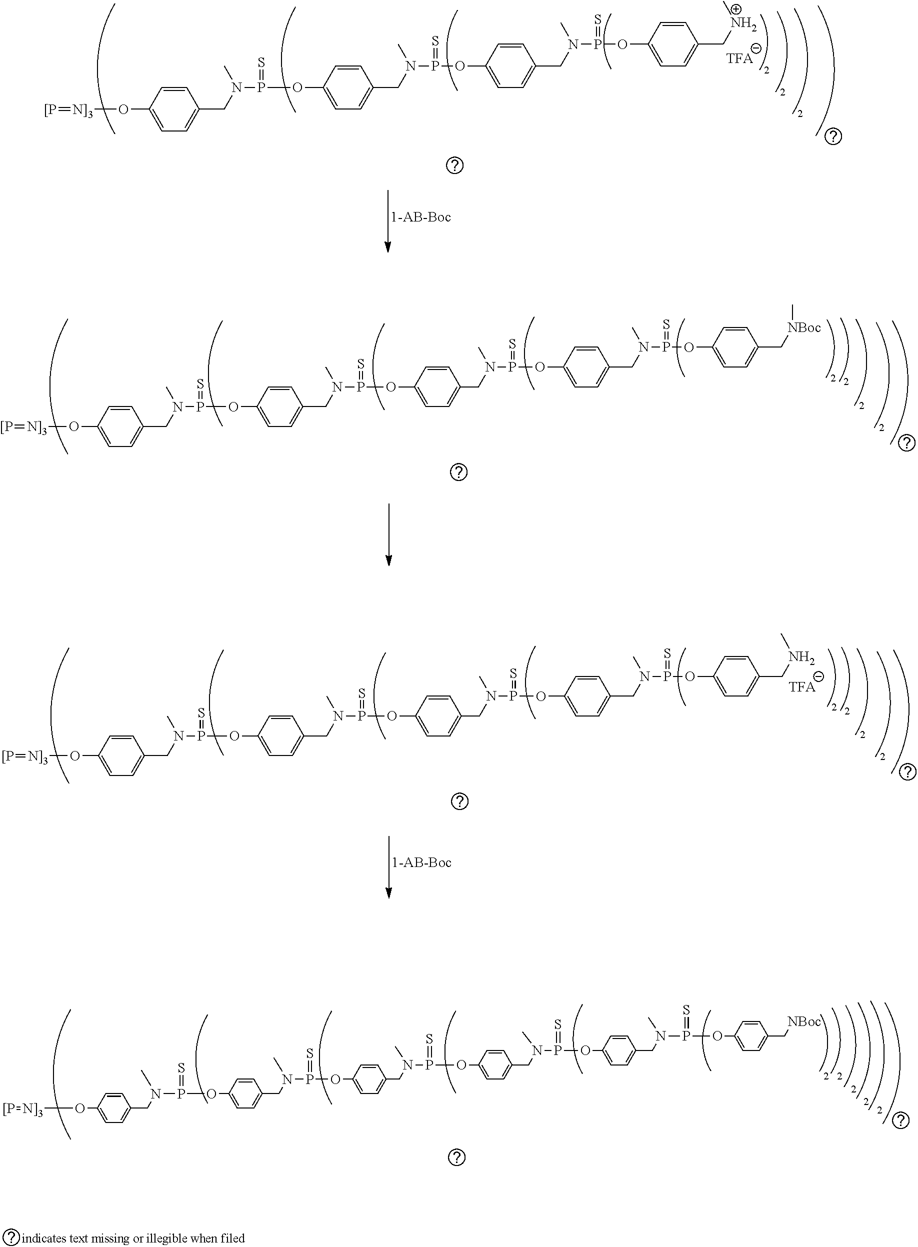

### General Procedure for Synthesis of Compounds 1-G′″ₙ-Boc from 1-G′″ₙ-1-HTFA with Synthon 1-AB₂-Boc

Compound AB₂-Boc (n=1, 0.633 mmol, n=2, 1.265 mmol, n=3, 2.531 mmol, n=4, 5.062 mmol, n=5, 10.13 mmol) is added to a solution of 200 mg of the trifluoroacetic acid salt of dendrimer 1-G′″₍ₙ₋₁₎-HTFA in CH₂Cl₂ (50 to 100 mL) with Et₃N (n=1, 0.696 mmol, n=2, 1.392 mmol, n=3, 2.784 mmol, n=4, 5.569 mmol, n=5, 11.14 mmol). After stirring overnight at ambient temperature, the mixture is concentrated to dryness. The yields are between 50 and 90%.

For n=1, 2 and 3, the crude residue is filtered over silica gel with an EtOAc/CH₂Cl₂ eluent gradient (5/95 to 30/70) to give compound 1-G′″ₙ-Boc in oil form.

For n=4, 5, the crude residue is washed 2 times with 10 mL of acetonitrile to give compound 1-G′″ₙ-Boc in powder form.

Characterization of 1-G′″₂-Boc:

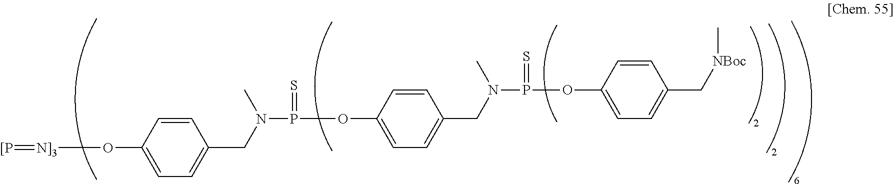

¹H NMR (600 MHz, CD₂Cl₂): δ (ppm) 7.40-6.91 (m, 168H, CH-arom), 4.63-4.47 (d, ³J_{HP}=12.6 Hz, 36H, CH₂N₀H, CH₂N₁H), 4.40 (s, 48H, CH₂N₂H), 3.14-2.84 (m, 54H, N₀—CH₃, N₁CH₃), 2.81 (s, 72H, N₂—CH₃), 1.47 (brs, 216H, Boc).

³¹P-{¹H} NMR (243 MHz, CD₂Cl₂): δ (ppm) 68.38 (s, P₂), 68.03 (s, P₁), 8.39 (s, P₀).

¹³C-{¹H} NMR (151 MHz, CD₂Cl₁₂): δ (ppm) 155.59 (d, NC(O)OtButyl), 150.43 (m, C₁¹, C₀¹), 150.06 (d, ³J_{CP}=7.5 Hz, C₂¹), 135.37 (s, C₂⁴), 134.28 (s, C₀⁴, C₁⁴), 129.41 (m, C₀³, C₁³) 128.56 (d, C₂³), 120.7-121.07 (m, C₀², C₁², C₂²), 79.34 (s, O—C-tButyl), 51.57 (d, CH₂N₂), 33.81 (s, N₂—CH₃), 33.64 (s, N₁—CH₃), 33.58 (s, N₀—CH₃), 28.14 (s, C(CH₃)₃).

### Characterization of 1-G′″₃-Boc

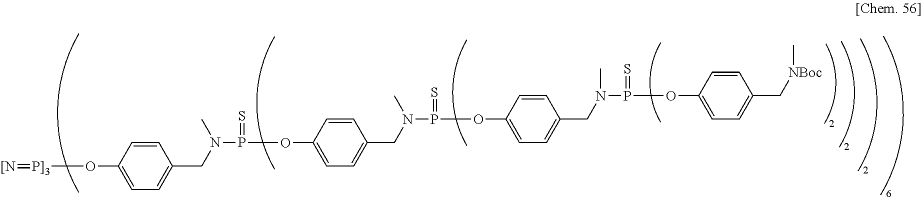

¹H NMR (600 MHz, CD₂Cl₂): δ (ppm) 7.41-6.97 (m, 10H, CH-arom), 4.49-4.65 (m, 10H, CH₂N₀H, CH₂N₁H, CH₂N₂H), 4.41 (s, 11H, CH₂N₃H), 3.01-2.88 (m, 1H, N₀—CH₃, N₁—CH₃, N₂—CH₃), 2.82 (s, 18H, N₃—CH₃), 1.49 (d, 50H, Boc).

³¹P-{¹H} NMR (243 MHz, CD₂Cl₁₂): δ (ppm) 68.42 (s, P₃), 68.14 (m, P₁, P₂), 8.28 (s, P₀).

¹³C-{¹H} NMR (151 MHz, CD₂Cl₁₂): δ (ppm) 155.64 (d, NC(O)OtButyl), 150.50 (m, C₁¹, C₀¹, C₂¹), 150.11 (m, C₃¹) 135.41 (s, C₃⁴), 134.30 (s, C₀⁴, C₁⁴, C₂⁴), 129.45 (s, C₀, C₁³, C₂³), 128.61 (s, C₃³), 121.40-120.71 (m, C₀², C₁², C₂², C₃²), 79.39 (s, O—C-tButyl), 51.72-51.62 (m, CH₂N), 33.86-33.61 (m, N₀—CH₃, N₁—CH₃, N₂—CH₃, N₃—CH₃), 28.19 (s, C(CH₃)₃).

### Characterization of 1-G′″₄-Boc

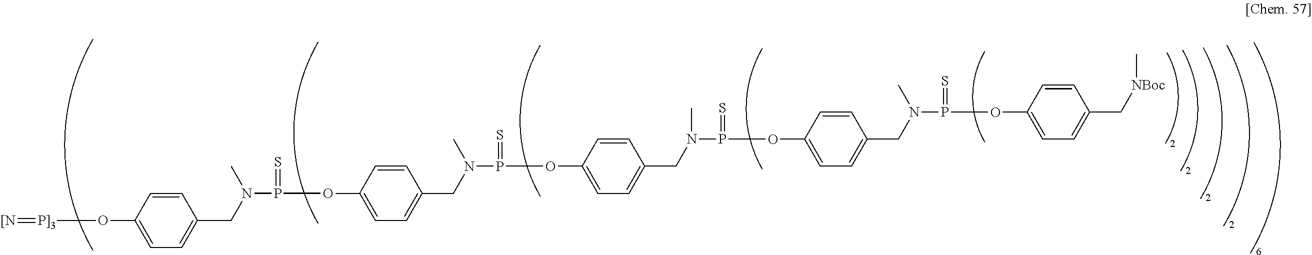

¹H NMR (400 MHz, CDCl₃): δ (ppm) 7.40-7.04 (m, 744H, CH-arom), 4.51-4.28 (m, 372H, CH₂N₀, CH₂N₁, CH₂N₂, CH₂N₃, CH₂N₄), 3.03-2.68 (m, 558H, N₀—CH₃, N₁—CH₃, N₂—CH₃, N₃—CH₃, N₄—CH₃), 1.46 (s, 864H, Boc).

³¹P-{¹H} NMR (162 MHz, CDCl₃): δ (ppm) 68.16 (s, P₄), 67.94 (m, P₁ P₂,P₃), 7.85 (s, P₀).

¹³C-{¹H} NMR (101 MHz, CDCl₃): δ (ppm) 155.84 (d, NC(O)OtButyl), 150.62-150.27 (m, C₁¹, C₀¹, C₂¹, C₃¹), 150.09 (d, ²J_{CP}=7.5 Hz, C₄¹), 135.00 (s, C₄⁴), 134.30 (d, ³J_{CP}=3.7 Hz, C₀⁴, C₁⁴, C₂⁴, C₃⁴), 129.47 (m, C₀³, C₁³, C₂³, C₃³), 128.63 (m, C₄³), 121.11 (d, ³J_{CP}=4.3 Hz, C₀², C₁², C₂², C₃², C₄²), 79.70 (s, O—C-tButyl), 53.64-53.58 (m, CH₂N₁, CH₂N₂, CH₂N₃) 52.31-50.86 (m, CH₂N₄) 33.94-33.75 (m, N₀—CH₃, N₁—CH₃, N₂—CH₃, N₃—CH₃, N₃—CH₃), 28.46 (s, C(CH₃)₃).

### Characterization of 1-G′″₅-Boc

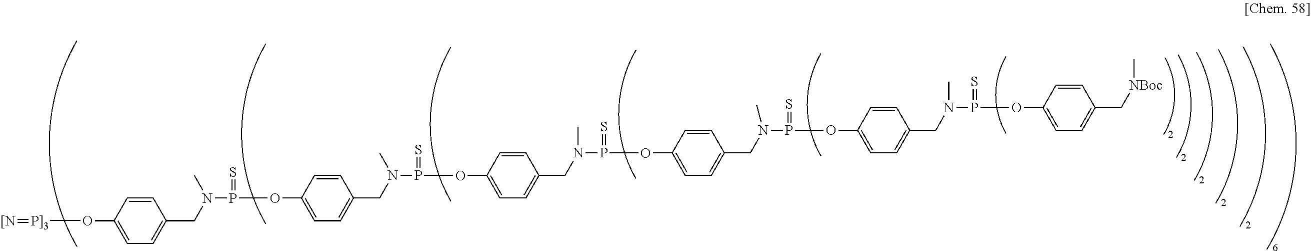

¹H NMR (300 MHz, CDCl₃): δ (ppm) 7.45-6.92 (m, 1512H, CH-arom), 4.43 (m, 756H, CH₂N₀, CH₂N₁, CH₂N₂, CH₂N₃, CH₂N₄, CH₂N₅), 2.81 (m, 1134H, N₀—CH₃, N₁—CH₃, N₂—CH₃, N₃—CH₃, N₄—CH₃, N₅—CH₃), 1.45 (s, 1728H, Boc).

³¹P-{¹H} NMR (162 MHz, CDCl₃): δ (ppm) 68.16 (s, P₄), 67.94 (m, P₁ P₂, P₃), 7.85 (s, P₀).

### General Synthesis Procedure for Dendrimers 1-G′″ₙ-HTFA from 1-G′″ₙ-Boc

Compound 1-G′″ₙBoc (200 mg) is dissolved in a CH₂Cl₂/trifluoroacetic acid 70/30 mixture (30 mL). After 30 min of stirring at ambient temperature, the mixture is concentrated to dryness under reduced pressure. The residue is subsequently co-evaporated 5 times with 3 mL of CH₂Cl₂ to give dendrimer 1-G′″ₙ-HTFA in the form of a white powder. The yields are greater than 90%.

### Characterization of 1-G′″₁-HTFA

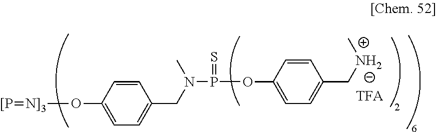

¹H NMR (400 MHz, CD₃CN) δ (ppm): 2.64 (s), 2.77 (d, J=10.8 Hz), 4.11 (s), 4.47 (d, J=13.4 Hz), 6.93 (d, J=8.4 Hz), 7.23 (d, J=9.7 Hz), 7.47 (d, J=8.6 Hz).

³¹P-{¹H} NMR (162 MHz, CD₃CN) δ (ppm): 9.09 (s, P0), 67.80 (s, P1). The structure of dendrimer 1-G′″₁-HTFA is represented in [FIG. 2].

### Characterization of 1-G′″₂-HTFA

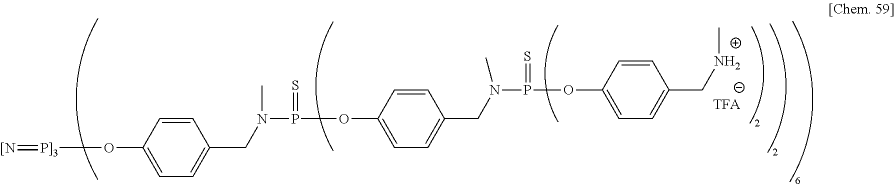

¹H NMR (600 MHz, CD₂Cl₂): δ (ppm) 7.40-6.91 (m, 168H, CH-arom), 4.63-4.47 (d, ³J_{HP}=12.6 Hz, 36H, CH₂N₀H, CH₂N₁H), 4.40 (s, 48H, CH₂N₂H), 3.14-2.84 (m, 54H, N₀—CH₃, N₁CH₃), 2.81 (s, 72H, N₂—CH₃), 1.47 (brs, 216H, Boc).

³¹P-{¹H} NMR (121 MHz, CD₃CN) δ (ppm): 9.12 (s, P0), 67.74 (s, P2), 68.38 (s, P1).

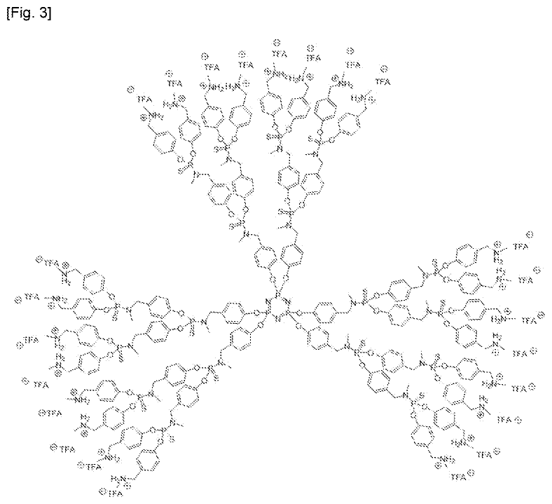

The structure of dendrimer 1-G′″₂-HTFA is represented in [FIG. 3].

### Characterization of 1-G′″₃-HTFA

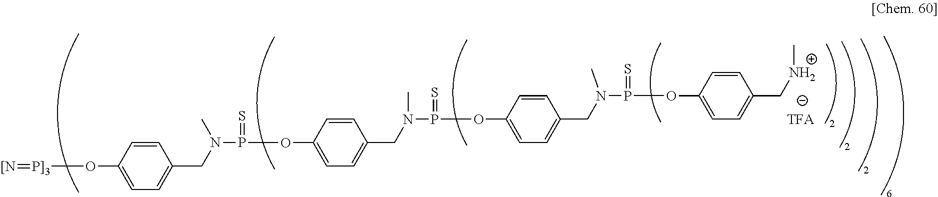

³¹P-{¹H} NMR (162 MHz, CD₃CN) δ (ppm): 9.10 (s, P0), 67.71 (s, (P3), 68.33 (s, P1, P2).

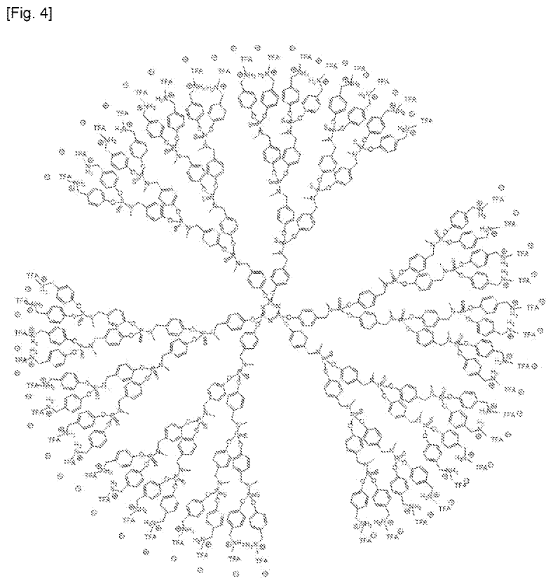

The structure of dendrimer 1-G′″₃-HTFA is represented in [FIG. 4].

### Characterization of 1-G′″₄-HTFA

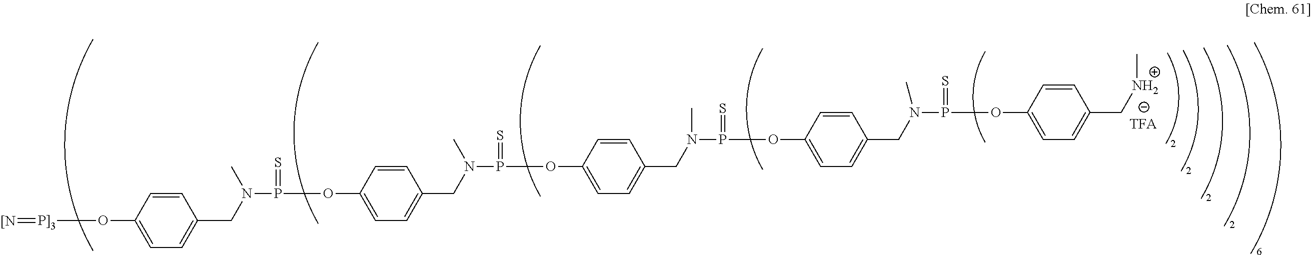

³¹P-{¹H} NMR (162 MHz, CD₃CN) δ (ppm): 8.97 (s, P0), 67.72 (s, P4), 68.30 (s, P1, P2, P3).

¹H NMR (400 MHz, CD₃CN) δ (ppm): 2.58-2.74 (m, 558H, N₀—CH₃, N₁—CH₃, N₂—CH₃, N₃—CH₃, N₄—CH₃), 4.06-4.58 (m, 372, CH₂N₀, CH₂N CH₂N₂, CH₂N₃, CH₂N₄), 6.65-7.87 (m, 744H, CH-arom).

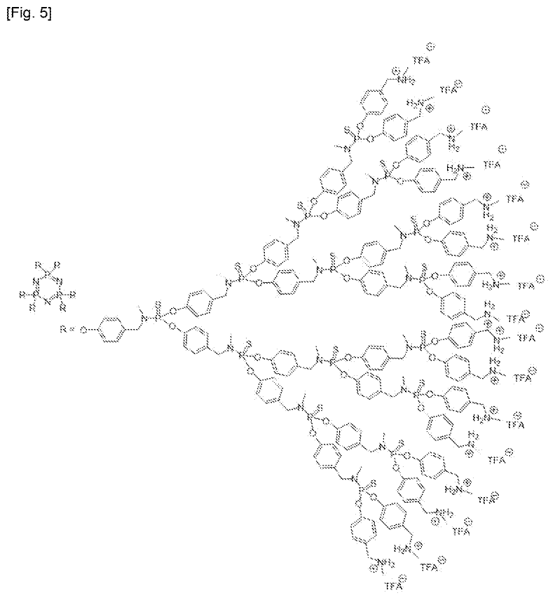

The structure of dendrimer 1-G′″₄-HTFA is represented in [FIG. 5].

### Divergent Synthesis of 1st Generation Dendrimers Having Bis-Phosphonate Terminations

Scheme 3 describes the reaction scheme for divergent synthesis of 1st generation dendrimers having bis-phosphonate terminations:

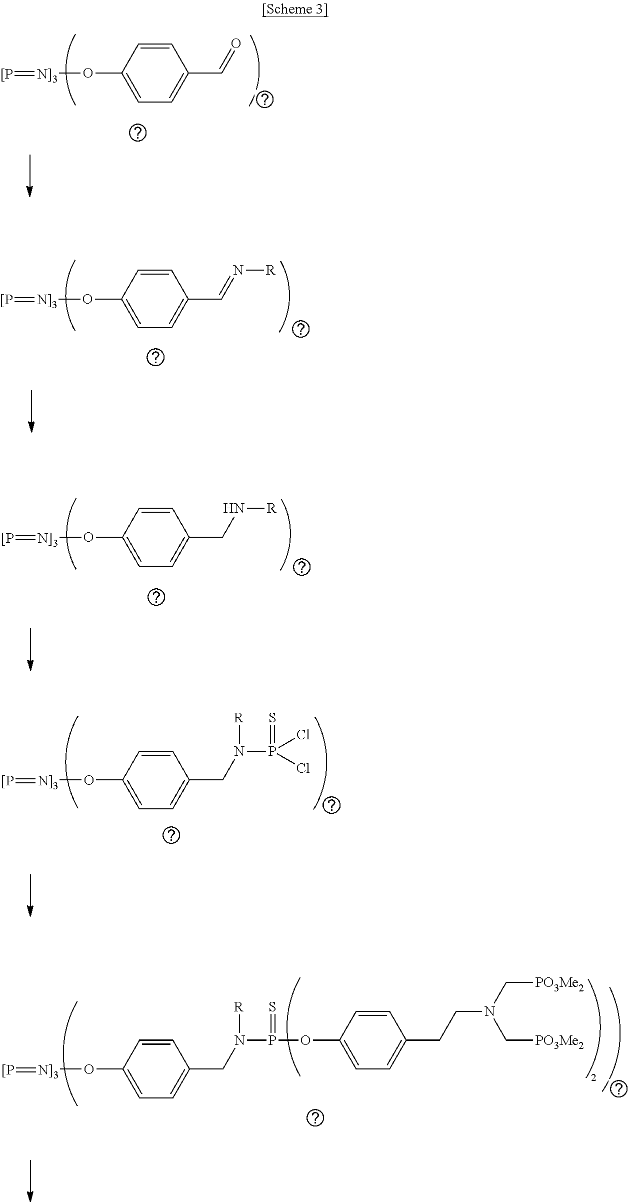

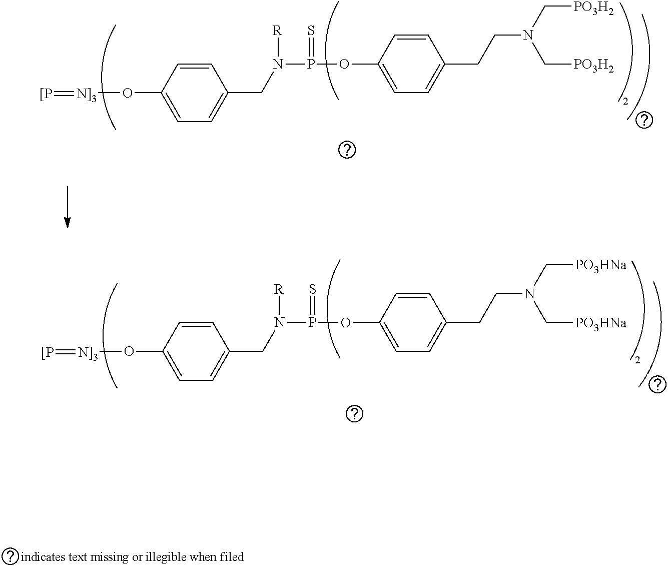

### Compound 3-G′₁ (OMe) from 1-G₁

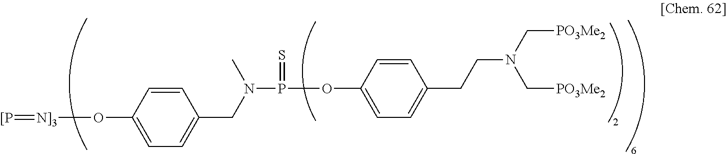

Phenol aminobismethylene phosphonate derived from tyramine (see WO 2005/052031 A1) (12 mmol) is added to a solution of compound 1-G₁ prepared previously (1 mmol) in THF (30 mL) with Cs₂CO₃ (24 mmol). After stirring overnight at ambient temperature, the mixture is centrifuged, filtered then concentrated under reduced pressure. The crude residue is diluted in minimal THF then washed in a large volume of diethyl ether. After drying under reduced pressure, dendrimer 3-G′₁(OMe) is obtained in the form of a transparent oil at a yield of greater than 95%.

³¹P-{¹H} NMR (243 MHz, CDCl₃) δ (ppm): 8.40 (s, N═P), 26.85 (s, P0), 68.32 (s, □S);

¹H NMR (600 MHz, CDCl₃) δ (ppm): 2.75 (d, ³J_{HH}=10.1 Hz, 24H, C₁⁴—CH₂), 2.77 (d, ³J_{HP}=7.6 Hz, 18H, CH₃), 3.06 (d, ³J_{HH}=7.7 Hz, 24H, C₁⁴—CH₂—CH₂), 3.19 (d, ²J_{HP}=9.4 Hz, 48H, CH₂—P), 3.73 (d, ³J_{HP}=10.6 Hz, 144H, POMe), 6.94 (d, ³J_{HH}=8.2 Hz, 12H, C₀²H), 7.07 (d, ³J_{HH}=8.1 Hz, 24H, C₁²H), 7.18 (br d, ³J_{HH}=8.2 Hz, 36H, C₀³ and C₁³H);

¹³C-{¹H} NMR (151 MHz, CDCl₃) δ (ppm): 33.05 (s, CH₂), 33.64 s, (CH₃N), 49.46 (dd, ¹J_{CP}=157.3, ³J_{CP}=7.2 Hz, CH₂—P), 53.56-51.75 (m, POCH₃), 53.47 (d, ²J_{CP}=10.3 Hz, C₁⁴—CH₂), 58.22 (t, ³J_{CP}=7.7 Hz, C₁⁴—CH₂—CH₂), 120.91 (br d, ³J_{CP}=4.4 Hz, C₀² and C₁²), 129.30 (s, C₀³), 129.87 (s, C₁³), 134.10 (br d, ²J_{CP}=4.5 Hz, C₀⁴), 136.22 (s, C₁⁴), 149.35 (d, ²J_{CP}=7.5 Hz, C₁¹), 150.01 (br s, C₀¹).

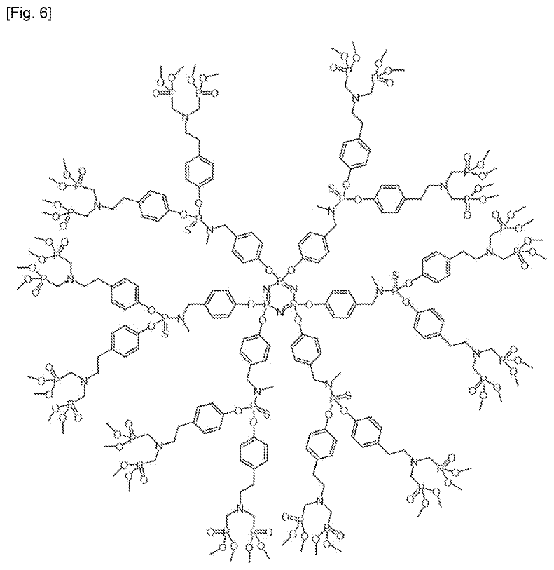

The structure of dendrimer 3-G′₁(OMe) is represented in [FIG. 6].

### Compound 3-G′₁ (OH) from 3-G′₁ (OMe)

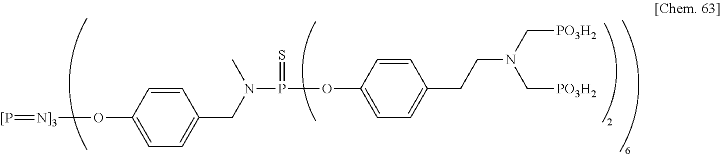

BrTMS (1.34, 10.2 mmol) is added dropwise to a solution of dendrimer 3-G′₁(OMe) (1 g, 0.17 mmol) in anhydrous CH₃CN (30 mL) at 0° C. After stirring overnight, the mixture is evaporated to dryness under reduced pressure. The residue is stirred for one hour in MeOH (10 mL), then washed 2 times with MeOH (20 mL) then once with Et₂O (20 mL). The solid obtained is dried under reduced pressure to give dendrimer 3-G′₁(OH) in the form of a white powder at a quantitative yield.

³¹P NMR (162 MHz, D₂O) δ (ppm): 7.36 (brs, POH), 8.35 (brs, POH), 68.53 (brs, PS).

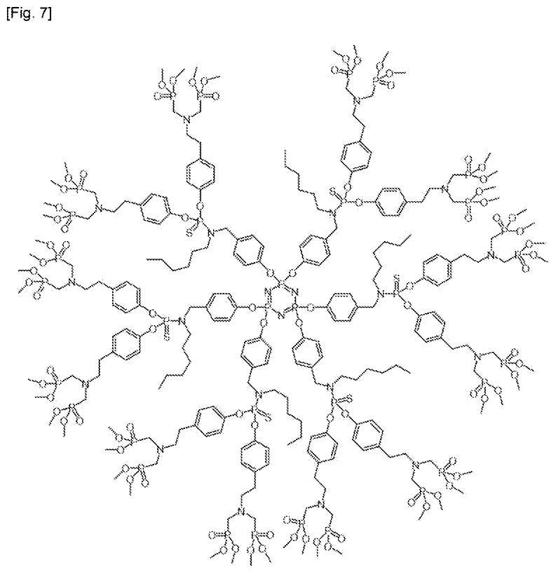

The structure of dendrimer 3-G′₁(OH) is represented in [FIG. 7].

The compound 3-G′₁(OH) is subsequently converted into 3-G′₁(Ona)

### Compound 3-G′₁(ONa) from 3-G′₁(OH)

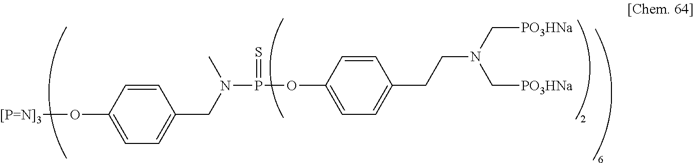

24 equivalents of a 0.1 M aqueous solution of NaOH are added to a suspension of 3-G′₁(OH) in water, with stirring. The resulting solution is filtered over a microfilter at 0.4 μM then lyophilized to give compound 3-G′₁(ONa) in the form of a white powder at a yield of 90%.

³¹P-{¹H} NMR (162 MHz, D₂O/CD₃CN) δ (ppm): 6.93 (s, PO), 9.59 (s, N═P), 68.77 (s, □S);

¹H NMR (600 MHz, D₂O/CD₃CN) δ (ppm): 2.76 (d, ³J_{HP}=10.7 Hz, 18H, CH₃), 3.13 (t, ³J_{HH}=8.8 Hz, 24H, C₁⁴—CH₂), 3.47 (d, ²J_{HP}=11.9 Hz, 48H, CH₂—P), 3.72 (t, ³J_{HH}=8.6 Hz, 24H, C₁⁴—CH₂—CH₂), 4.47 (d, ²J_{HP}=13.5 Hz, 12H, C₀⁴—CH₂), 6.91 (d, ²J_{HH}=8.1 Hz, 12H, C₀²H), 7.15 (d, ²J_{HH}=8.1 Hz, 24H, C₁²H), 7.29 (d, ²J_{HH}=8.3 Hz, 12H, C₀³H), 7.38 (d, ²J_{HH}=8.2 Hz, 24H, C₁³H);

¹³C-{¹H} NMR (151 MHz, D₂O/CD₃CN) δ (ppm): 29.07 (s, C₁⁴—CH₂), 33.58 (s, CH₃), 52.91 (d, ¹J_{CP}=127.6 Hz, CH₂—P), 52.94 (s, C₀⁴—CH₂), 57.81 (s, C₁⁴—CH₂—CH₂), 121.14 (br s, C₀²), 121.42 (d, ³J_{CP}=4.4 Hz C₁²), 129.61 (s, C₀³), 130.59 (s, C₁³), 133.95 (s, C₁⁴), 134.89 (s, C₀⁴), 149.18 (s, C₀¹), 149.60 (d, ³J_{CP}=7.6 Hz, C₁¹) ppm.

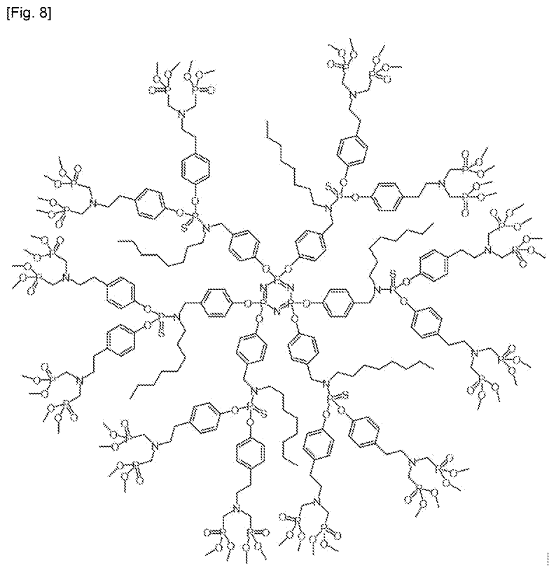

The structure of dendrimer 3-G′₁(ONa) is represented in [FIG. 8].

### Synthesis of compound 4-G″₀ from 1-G′₀

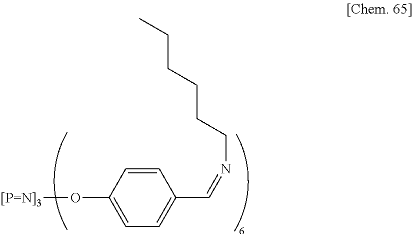

1-Aminohexane (5.5 mL, 41.8 mmol) is added to a solution of compound 1-G′₀ (2 g, 2.32 mmol) in THF (40 mL). After stirring overnight at ambient temperature, the mixture is concentrated to dryness then washed 3-4 times with MeOH to give the dendrimer 4-G″₀ in the form of a white powder at a quantitative yield.

³¹P-{¹H} NMR (121 MHz, CD₂Cl₂) δ (ppm): 8.46 (s, P═N).

¹H NMR (300 MHz, CD₂Cl₂) δ (ppm): 0.79-1.10 (m, 18H, —CH₃), 1.29-1.47 (m, 36H, —CH₂—), 1.68-1.77 (m, 12H, —CH₂—), 3.63 (td, ³J_{HH}=7.1, ⁴J_{HH}=1.3 Hz, 12H, —CH₂—N), 7.00 (d, ³J_{HH}=8.4 Hz, 12H, C₀²H), 7.58 (d, ³J_{HH}=8.7 Hz, 12H, C₀³H), 8.23 (s, 6H, CH═N).

### Compound 4-G′″₀ from 4-G″₀

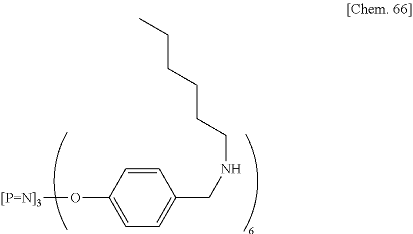

NaBH₄(672 mg, 17.75 mmol) is added to a solution of dendrimer 4-G″0 (2 g, 1.4 mmol) in a THF/MeOH (30/10) mL mixture. After stirring overnight at ambient temperature, the mixture is concentrated to dryness. The crude residue is diluted in 200 mL of DCM then washed once with 50 mL distilled water. The organic phase is recovered, dried with Na₂SO₄, filtered and concentrated under reduced pressure. The product is subsequently dissolved in MeOH (30 mL) to which 20 mL of HCl (1 M in MeOH) is added. After 2 h without stirring, the solution is filtered then concentrated under reduced pressure. The residue is subsequently washed 3 times with 25 mL of DCM to give a white powder. The product is dissolved in a DCM/THF mixture (75/25 mL), to which is added 25 mL of a solution of NaOH (2 M) then extracted 4-5 times with 200 mL of DCM. The organic phase is recovered, dried with Na₂SO₄, filtered and concentrated under vacuum to give dendrimer 4-G′″₀ in the form of a colorless oil at a yield of 78%.

³¹P-{¹H} NMR (121 MHz, CDCl₃) δ (ppm): 8.72 (s, P═N).

¹H NMR (300 MHz, CDCl₃) δ (ppm): 0.73-1.09 (m, 18H, CH₃—)), 1.13-1.42 (m, 48H, —CH₂—), 1.45-1.55 (m, 12H, —CH₂—), 2.60 (t, ³J_{HH}=7.2 Hz, 12H, —CH₂—N), 3.73 (s, 12H, C₀⁴—CH₂—), 6.90 (d, ³J_{HH}=8.4 Hz, 12H, C₀²H), 7.13 (d, ³J_{HH}=8.6 Hz, 12H, C₀³H).

### Compound 4-G₁ from 4-G′″₀

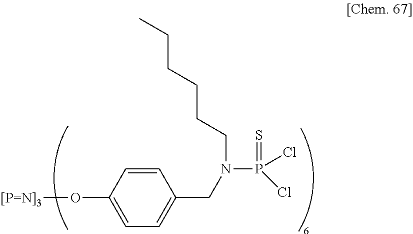

A solution of dendrimer 4-G′″₀ (1 g, 0.72 mmol) is added dropwise to a solution of PSCl₃ (4 mL, 39.36 mmol) in DCM (100 mL) at −70° C. in the presence of Et₃N (0.91 mL, 6.57 mmol) in DCM (40 mL) over a duration of 30 min. After stirring overnight, the reaction mixture is concentrated to dryness under reduced pressure. The crude residue is subsequently dissolved in 200 mL of DCM then filtered over silica gel to give dendrimer 4-G₁ in the form of a transparent oil at a yield of 35%.

³¹P-{¹H} NMR (162 MHz, CDCl₃) δ (ppm): 8.36 (s, P═N), 63.09 (s, PS).

¹H NMR (400 MHz, CDCl₃) δ (ppm): 0.89 (t, ³J_{HH}=6.9 Hz, 18H, —CH₃), 1.23-1.47 (m, 36H, —CH₂—), 1.57-1.64 (m, 12H, —CH₂—), 3.23 (dt, ³J_{HP}=18.0, ³J_{HH}=7.8 Hz, 12H, —CH₂—N), 4.63 (d, ³J_{HP}=16.9 Hz, 12H, C₀⁴—CH₂—), 6.99 (d, ³J_{HH}=8.4 Hz, 12H, C₀²H), 7.23 (d, ³J_{HH}=8.5 Hz, 12H, C₀³H).

¹³C-{¹H} NMR (101 MHz, CDCl₃) δ (ppm): 14.01 (s, CH₃—), 22.53 (s, —CH₂—), 26.33 (s, —CH₂—), 26.90 (d, ²J_{CP}=2.9 Hz, —CH₂—N), 31.36 (s, —CH₂—), 47.38 (s, —CH₂—), 50.11 (d, ²J_{CP}=4.8 Hz, C₀⁴—CH₂—), 121.22 (br s, C₀²), 129.16 (s, C₀³), 132.67 (d, ³J_{CP}=5.0 Hz, C₀⁴), 150.22 (dd, ²J_{CP}=5.1, ⁴J_{CP}=2.5 Hz, C₀¹)

### Compound 4-G′₁ (OMe) from 4-G₁

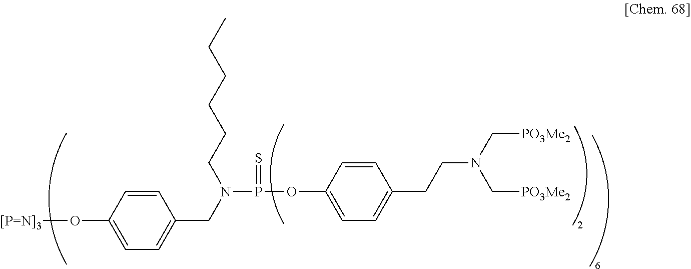

Phenol aminobismethylene phosphonate derived from tyramine (see WO 2005/052031 A1) (12 mmol) is added to a solution of compound 4-G₁ (1 mmol) in THF (30 mL) with Cs₂CO₃ (24 mmol). After stirring overnight at ambient temperature, the mixture is centrifuged, filtered then concentrated under reduced pressure. The crude residue is diluted in minimal THF then washed in a large volume of diethyl ether. After drying under reduced pressure, dendrimer 4-G′₁(OMe) is obtained in the form of a transparent oil at a quantitative yield.

³¹P-{¹H} NMR (162 MHz, CDCl₃) δ (ppm): 8.23 (s, P═N), 26.89 (s, POMe), 68.00 (s, PS).

¹H NMR (400 MHz, CDCl₃) δ (ppm): 0.78 (t, ³J_{HH}=6.6 Hz, 18H, CH₃—), 1.10-1.26 (m, 36H, —CH₂—), 1.41-1.47 (m, 12H, —CH₂—), 2.75 (t, ³J_{HH}=7.6 Hz, 24H, —CH₂-C₁⁴), 3.04 (t, ³J_{HH}=7.7 Hz, 24H, —CH₂—N), 3.09-3.15 (m, 12H, —CH₂—NP), 3.19 (d, ²J_{HP}=9.3 Hz, 18H, —CH₂—P), 3.65-3.79 (m, 144H, POMe), 4.53 (d, ³J_{HP}=13.1 Hz, 12H, —CH₂-C₀⁴), 6.95 (d, ³J_{HH}=8.1 Hz, 12H, C₀²H), 7.06 (d, ³J_{HH}=8.2 Hz, 24H, C₁²H), 7.16 (d, ³J_{HH}=8.2 Hz, 24H, C₁²H), 7.22 (d, ³J_{HH}=8.3 Hz, 24H, C₀³H).

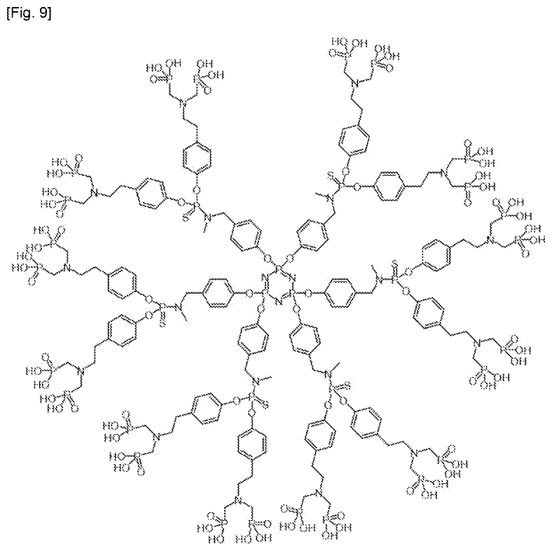

The structure of dendrimer 4-G′₁(OMe) is represented in [FIG. 9].

### Compound 4-G′₁ (OH) from 4-G′₁(OMe)

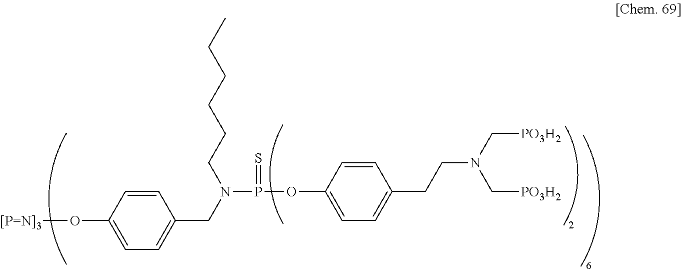

BrT□S (1.25, 9.48 mmol) is added to a solution of dendrimer 4-G₁ (OMe) (1 g, 0.16 mmol) in anhydrous CH₃CN (30 mL) at 0° C. After stirring overnight, the mixture is evaporated to dryness under reduced pressure. The residue is stirred for one hour in □eOH (10 mL), then washed 2 times with □eOH (20 mL) then once with Et₂O (20 mL). The solid obtained is dried under reduced pressure to give dendrimer 4-G₁(OH) in the form of a white powder at a quantitative yield.

³¹ □-{¹H} N□R (162 □Hz, D₂O) δ (ppm): 8.20 (brs, □OH)

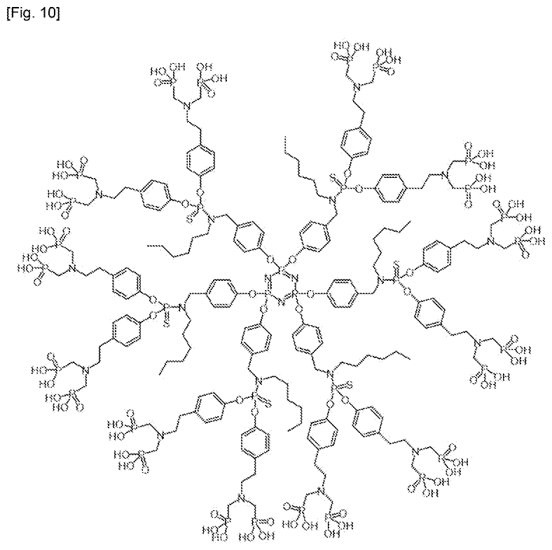

The structure of dendrimer 4-G′₁(OH) is represented in [FIG. 10].

The compound 4-G′₁(OH) is subsequently converted into 4-G′₁(ONa)

### Compound 4-G′₁ (ONa) from 4-G′₁ (OH)

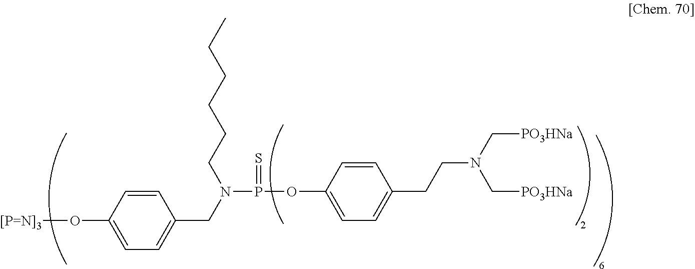

24 equivalents of a 0.1□ aqueous solution of NaOH are added to a suspension of 4-G′₁(OH) in water, with stirring. The resulting solution is filtered over a microfilter at 0.4 μM then lyophilized to give compound 4-G′₁(ONa) in the form of a white powder at a yield of 88%.

³¹□-{¹H} N□R (162 □Hz, D₂O) δ (ppm) 6.64 (s, □OHONa), 9.50 (s, □=N), 68.66 (s, □S).

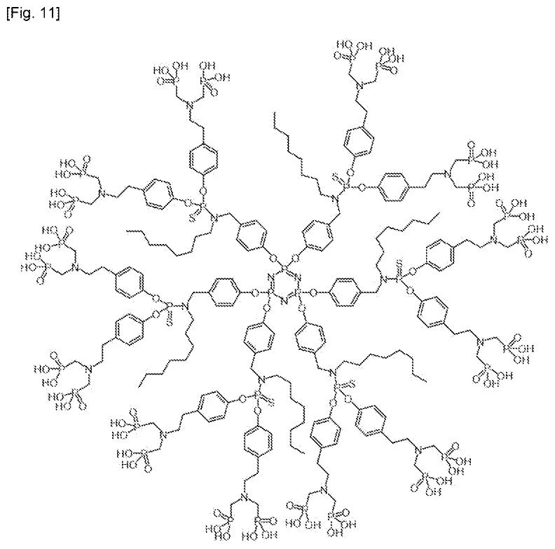

The structure of dendrimer 4-G′₁(ONa) is represented in [FIG. 11].

### Compound 5-G″₀ from 1-G′₀

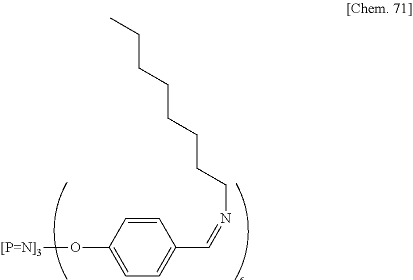

1-Aminooctane (6.8 mL, 41.8 mmol) is added to a solution of compound 1-G′₀ (2 g, 2.32 mmol) in THF (40 mL). After stirring overnight at ambient temperature, the mixture is concentrated to dryness then washed 3-4 times with □eOH to give the dendrimer 5-G″₀ in the form of a white powder at a quantitative yield.

³¹□-{¹H} N□R (121 □Hz, CD₂Cl₂) δ (ppm): 8.46 (s, □=N).

¹H N□R (300 □Hz, CD₂Cl₂) δ (ppm): 0.73-1.13 (m, 18H, CH₃—), 1.19-1.45 (m, 60H, —CH₂—), 1.78-1.66 (m, 12H, —CH₂—), 3.62 (td, ³J_{HH} 7.1, ⁴J_{HH} 1.3 Hz, 12H, CH₂N), 6.99 (d, ³J_{HH} 8.6 Hz, 12H, C₀²H), 7.58 (d, ³J_{HH} 8.7 Hz, 12H, C₀³H), 8.22 (s, 6H, CH═N).

¹³C-{¹H} NMR (75 MHz, CD₂Cl₂) δ (ppm): 13.86 (s, CH₃—), 22.66 (s, —CH₂—), 27.43 (s, —CH₂—), 29.31 (s, —CH₂—), 29.47 (s, —CH₂—), 31.04 (s, —CH₂—), 31.88 (s, —CH₂—), 61.67 (s, —CH₂N), 120.93 (d, ³J_{CP}=2.9 Hz, C₀²H), 129.19 (s, C₀³H), 133.78 (s, C₀⁴), 151.80 (d, ²J_{CP}=7.6 Hz, C₀¹), 158.93 (s, —C═N—).

### Compound 5-G′″₀ from 5-G″₀

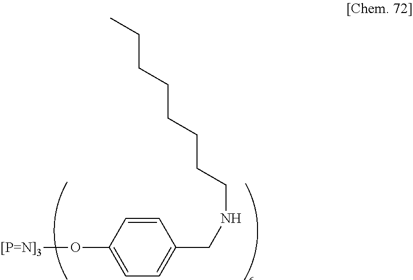

NaBH₄ (594 mg, 15.68 mmol) is added to a solution of dendrimer 5-G″₀ (2 g, 1.3 mmol) in a THF/MeOH (30/10) mL mixture. After stirring overnight at ambient temperature, the mixture is concentrated to dryness. The crude residue is diluted in 200 mL of DCM then washed once with 50 mL distilled water. The organic phase is recovered, dried with Na₂SO₄, filtered and concentrated under reduced pressure. The product is subsequently dissolved in MeOH (30 mL) to which 20 mL of HCl (1 M in MeOH) is added. After 2 h without stirring, the solution is filtered then concentrated under reduced pressure. The residue is subsequently washed 3 times with 25 mL of DCM to give a white powder. The product is dissolved in a DCM/THF mixture (75/25 mL), to which is added 25 mL of a solution of NaOH (2 M) then extracted 4-5 times with 200 mL of DCM. The organic phase is recovered, dried with Na₂SO₄, filtered and concentrated under vacuum to give dendrimer 5-G′₀ in the form of a colorless oil at a yield of 65%.

³¹P-{¹H} NMR (121 MHz, CD₂Cl₂) δ (ppm): 8.92 (s, P═N).

¹H NMR (300 MHz, CD₂Cl₂) δ (ppm): 0.70-1.13 (m, 18H, CH₃—), 1.23-1.45 (m, 60H, —CH₂—), 1.46-1.66 (m, 12H, —CH₂—), 2.64 (t, ³J_{HH}=7.1 Hz, 12H, —CH₂N), 3.77 (s, 12H, C₀⁴—CH₂—), 6.91 (d, ³J_{HH}=8.4 Hz, 12H, C₀²H), 7.22 (d, ³J_{HH}=8.5 Hz, 12H, C₀³H).

¹³C-{¹H} NMR (75 MHz, CD₂Cl₂) δ (ppm): 13.88 (s, CH₃—), 22.68 (s, —CH₂—), 27.41 (s, —CH₂—), 29.33 (s, —CH₂—), 29.60 (s, —CH₂—), 30.11 (s, —CH₂—), 31.88 (s, —CH₂—), 49.55 (s, —CH₂—), 53.31 (s, —CH₂—), 115.12-124.47 (m, C₀²), 129.03 (s, C₀³), 137.59 (s, C₀⁴), 149.37 (dd, ²J_{CP}=5.1, ⁴J_{CP}=2.5 Hz, C₀¹).

### Compound 5-G₁ from 5-G′″₀

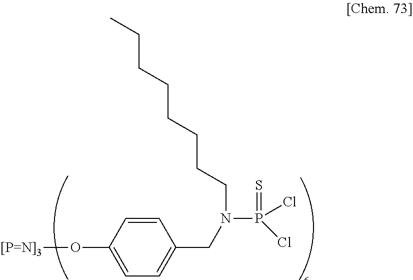

A solution of dendrimer 5-G′″₀ (1 g, 0.64 mmol) is added dropwise to a solution of PSCl₃ (4 mL, 39.36 mmol) in DCM (100 mL) brought to −70° C. in the presence of Et₃N (0.81 mL, 5.84 mmol) in DCM (40 mL) over a duration of 30 min. After stirring overnight, the reaction mixture is concentrated to dryness under reduced pressure. The crude residue is subsequently dissolved in 200 mL of DCM then filtered over silica gel to give dendrimer 5-G₁ in the form of a transparent oil at a yield of 25%.

³¹P-{¹H} NMR (162 MHz, CD₂Cl₂) δ (ppm): 8.42 (s, P═N), 63.04 (s, PS).

¹H NMR (400 MHz, CD₂Cl₂) δ (ppm): 0.91 (t, ³J_{HH}=6.7 Hz, 18H, —CH₃), 1.14-1.40 (m, 72H, —CH₂—), 1.59-1.68 (m, 24H, —CH₂—), 3.00-3.50 (m, 12H, —CH₂—N), 4.67 (d, ³J_{HP}=17.0 Hz, 12H, C₀⁴—CH₂—), 7.02 (d, ³J_{HH}=8.3 Hz, 12H, C₀²H), 7.29 (d, ³J_{HH}=8.4 Hz, 12H, C₀³H).

### Compound 5-G′₁ (OMe) from 5-G₁

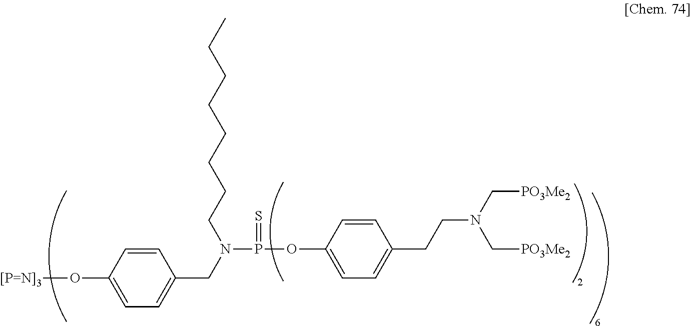

Phenol aminobismethylene phosphonate derived from tyramine (see WO 2005/052031 A1) (12 mmol) is added to a solution of compound 5-G₁ (1 mmol) in THF (30 mL) with Cs₂CO₃ (24 mmol). After stirring overnight at ambient temperature, the mixture is centrifuged, filtered then concentrated under reduced pressure. The crude residue is diluted in minimal THF then washed in a large volume of diethyl ether. After drying under reduced pressure, dendrimer 5-G′₁(OMe) is obtained in the form of a transparent oil at a quantitative yield.

³¹P-{¹H} NMR (162 MHz, CDCl₃) δ (ppm): 8.22 (s, P═N), 26.89 (s, POMe), 68.01 (s, PS).

¹H NMR (400 MHz, CDCl₃) δ (ppm): 0.83 (t, ³J_{HH}=7.0 Hz, 18H, CH₃), 1.01-1.41 (m, 60H, CH₂), 1.45 (br s, 12H, CH₂), 2.77 (t, ³J_{HH}=7.5 Hz, 24H, C₁⁴—CH₂—), 3.06 (t, ³J_{HH}=7.7 Hz, 24H, CH₂N), 3.09-3.15 (m, 12H, CH₂NPS), 3.20 (d, ²J_{HP}=9.3 Hz, 48H, CH₂P), 3.74 (d, ³J_{HP}=10.6, 1.5 Hz, 72H, POMe), 3.75 (d, ³J_{HP}=10.6, 1.5 Hz, 72H, POMe), 4.55 (d, ³J_{HP}=13.1 Hz, 12H, C₀⁴—CH₂—), 6.96 (d, ³J_{HH}=8.2 Hz, 12H, C₀²H), 7.02-7.12 (m, 24H, C₁²H), 7.18 (d, ³J_{HH}=8.5 Hz, 24H, C₁³H), 7.24 (d, ³J_{HH}=8.4 Hz, 12H, C₀³H).

¹³C-{¹H} NMR (101 MHz, CDCl₃) δ (ppm): 14.11 (s, CH₃), 22.61 (s, CH₂), 26.81 (s, CH₂), 27.62 (s, CH₂), 29.18 (s, CH₂), 31.72 (s, CH₂), 33.07 (s C₁⁴—CH₂—), 45.74 (br s, CH₂NS), 49.45 (dd, ¹J_{CP}=157.2, ³J_{CP}=7.1 Hz, CH₂P), 49.46 (br s, C₀⁴—CH₂—), 52.59-52.71 (m, POMe), 58.30 (t, ³J_{CP}=7.7 Hz, CH₂N), 120.90 (s, C₀²H), 121.02 (d, ²J_{CP}=4.7 Hz, C₀²H), 129.39 (s, C₀³H), 129.77 (s, C₁³H), 134.28 (s, C₀⁴), 136.09 (s, C₁⁴), 149.54 (d, ²J_{CP}=7.8 Hz, C₁¹), 150.00 (d, ²J_{CP}=4.5 Hz, C₀¹).

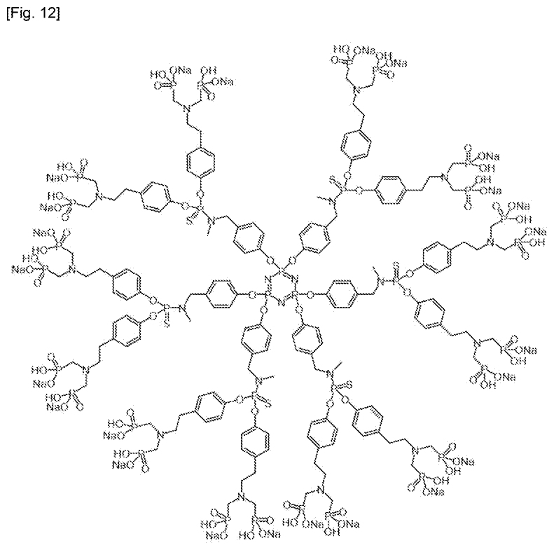

The structure of dendrimer 5-G′₁(OMe) is represented in [FIG. 12].

### Compound 5-G′₁(OH) from 5-G′₁(OMe)

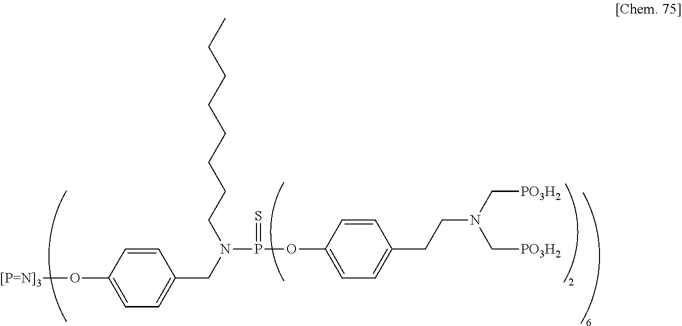

BrTMS (1.22, 9 mmol) is added to a solution of dendrimer 5-G₁(OMe) (1 g, 0.15 mmol) in anhydrous CH₃CN (30 mL) at 0° C. After stirring overnight, the mixture is evaporated to dryness under reduced pressure. The residue is stirred for one hour in MeOH (10 mL), then washed 2 times with MeOH (20 mL) then once with Et₂O (20 mL). The solid obtained is dried under reduced pressure to give dendrimer 5-G₁(OH) in the form of a white powder at a quantitative yield.

³¹P-{¹H} NMR (162 MHz, D₂O) δ (ppm): 7.19 (brs, POH).

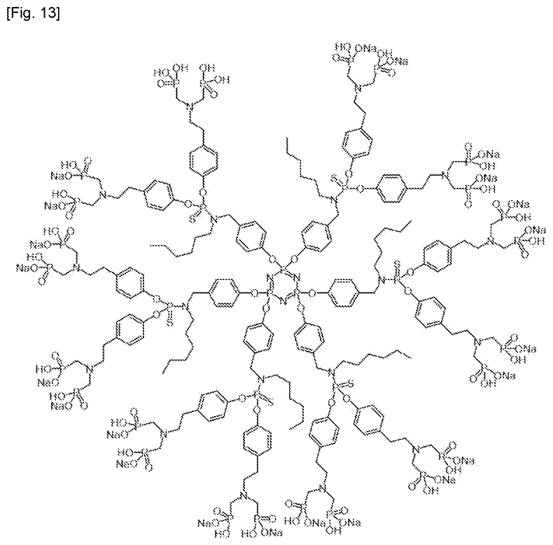

The structure of dendrimer 5-G′₁(OH) is represented in [FIG. 13].

The compound 5-G′₁(OH) is subsequently converted into 5-G′₁(ONa)

### Compound 5-G′₁ (ONa) from 5-G′₁ (OH)

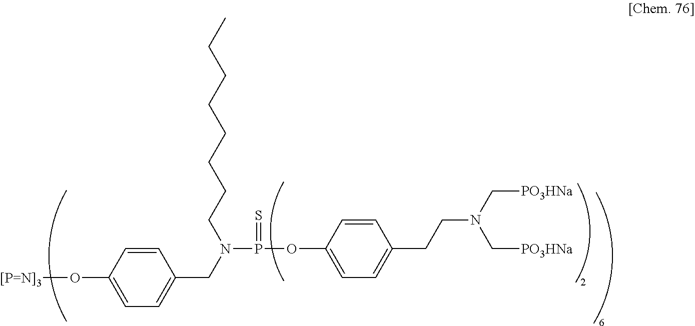

24 equivalents of a 0.1 M aqueous solution of NaOH are added to a suspension of 5-G′₁(OH) in water, with stirring. The resulting solution is filtered over a microfilter at 0.4 μM then lyophilized to give compound 5-G′₁(ONa) in the form of a white powder at a yield of 85%.

³¹P-{¹H} NMR (243 MHz, D₂O) δ (ppm): 6.64 (s, N═P), 9.50 (s, PO), 68.66 (s, PS).

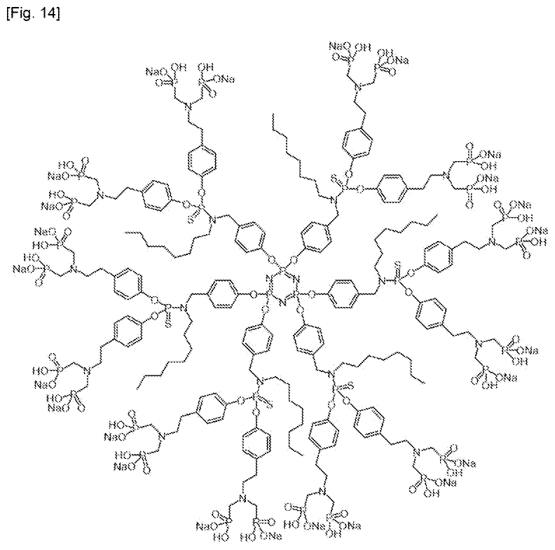

The structure of dendrimer 5-G′₁(ONa) is represented in [FIG. 14].

### Semi-Convergent Synthesis of 1st Generation Dendrimers Having Bisphosphonate Terminations

Scheme 4 describes the reaction scheme for divergent synthesis of 1st generation dendrimers having bis-phosphonate terminations:

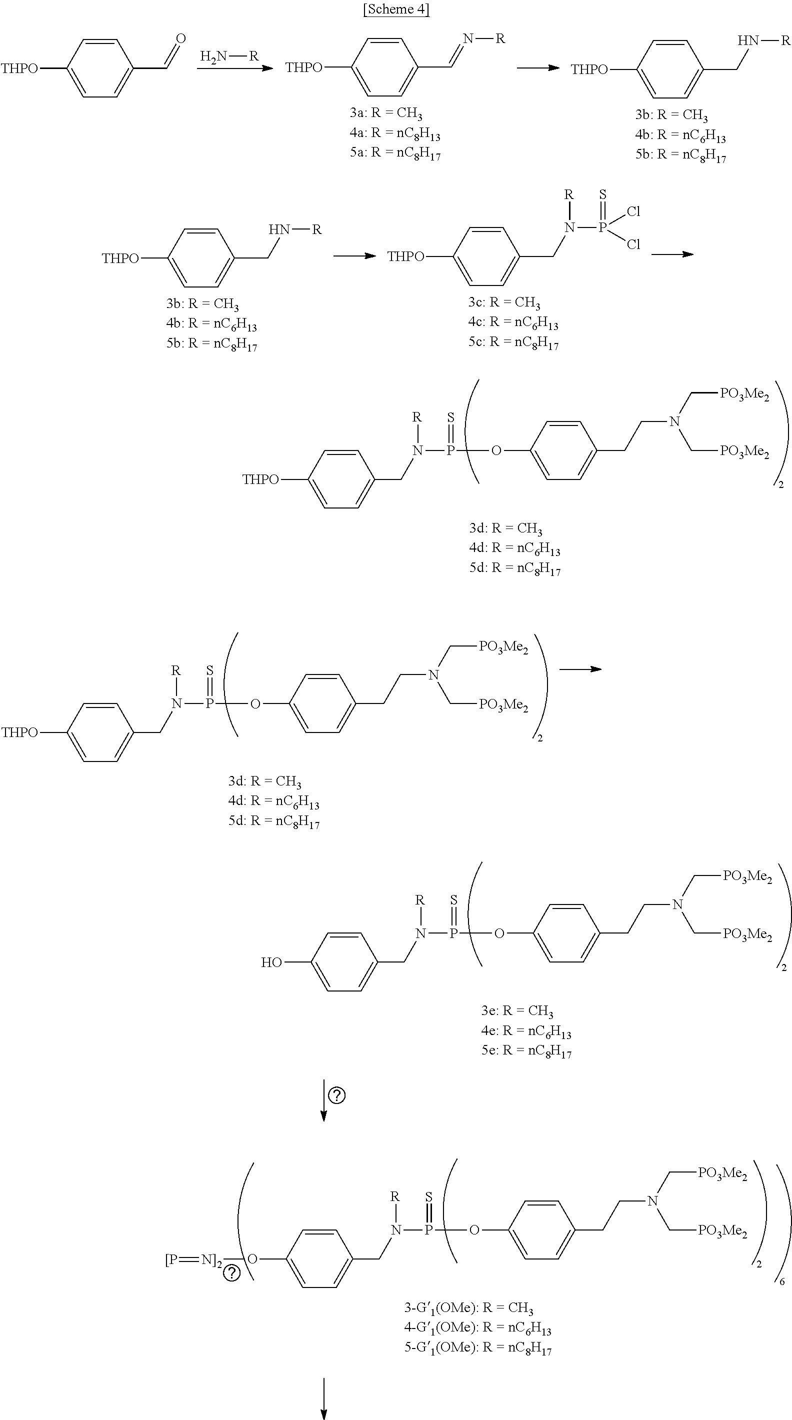

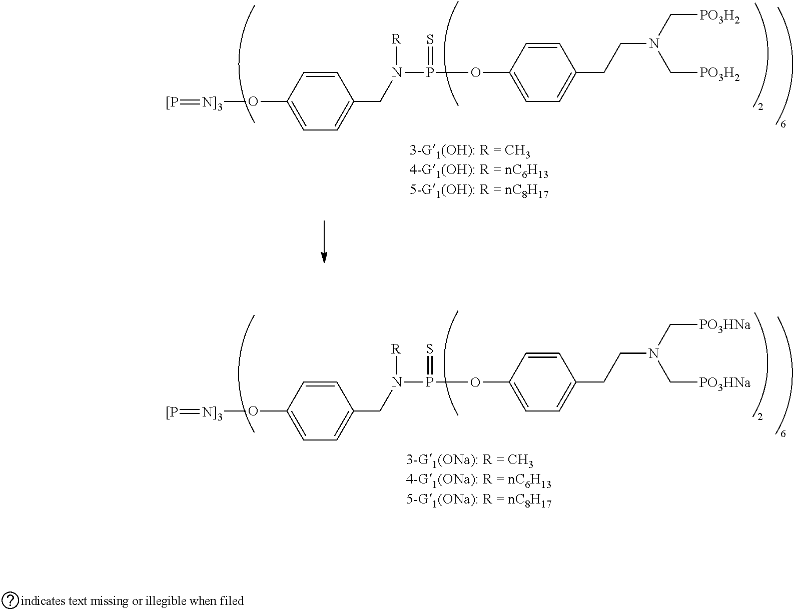

Compound 3a

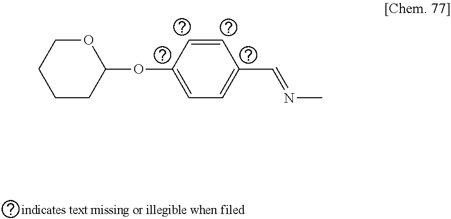 [SMILES: C/N=C/c1ccc(OC2CCCCO2)cc1]

To a solution of 4-((tetrahydro-2H-pyran-2-yl)oxy)benzaldehyde (Fu H. et al. Molecules, 2014, 16, 17715-17726) (1 g, 4.83 mmol) in THF (30 mL) a solution of methylamine (8M in EtOH) (1.8, 14.49 mmol) is added. After stirring overnight at ambient temperature, the mixture is concentrated to dryness to give the product 3a in the form of a colorless oil at a yield of 95%.

¹H NMR (300 MHz, CD₃OD) δ (ppm): 1.39-1.74 (m, 3H, —CH₂—), 1.75-1.92 (m, 2H, —CH₂—), 1.90-2.06 (m, 1H, —CH₂—O), 3.55-3.62 (m, 1H, —CH₂—O), 3.79-3.87 (m, 1H, —CH₂—), 5.46 (t, ³J_{HH}=3.1 Hz, 1H, O—CH—O), 7.08 (d, ³J_{HH}=8.8 Hz, 2H, C₀²H), 7.63 (d, ³J_{HH}=8.8 Hz, 2H, C₀³H), 8.21 (q, ⁴J_{HH}=1.6 Hz, 1H, —CH═N—).

¹³C-{¹H} NMR (75 MHz, CD₃OD) δ (ppm): 18.46 (s, —CH₂—), 24.89 (s, —CH₂—), 29.95 (s, —CH₂—), 46.32 (s, CH₃—), 61.73 (s, —CH₂—O), 96.10 (s, —CH—O), 116.17 (s, C₀²), 129.22 (s, C₀⁴), 129.30 (s, C₀³), 159.41 (s, C₀⁴), 163.46 (s, C═N).

Compounds 3b

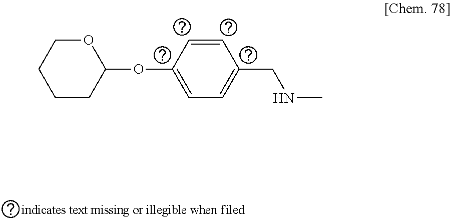 [SMILES: CNCc1ccc(OC2CCCCO2)cc1]

Pd/C at 10% (225 mg) and MeOH (40 mL) are introduced to a solution of compound 3a (1 g, 4.5 mmol) in a Fisher-Porter type tube. The tube is slowly placed under vacuum so as to evacuate the air. The H2 pressure is subsequently adjusted to 5 bar. The reaction mixture is stirred for 2 hours at ambient temperature. After slow depressurization, the reaction mixture is recovered, filtered and concentrated under reduced pressure. The residue is purified by silica gel chromatography (eluent: EtOAc) to obtain compound 3b in the form of a transparent oil at a yield of 80%.

¹H NMR (300 MHz, CDCl₃) δ (ppm): 1.33-1.72 (m, 3H, —CH₂—), 1.77-1.82 (m, 2H, —CH₂—), 1.87-2.07 (m, 1H, —CH₂—O), 2.36 (s, 3H, CH₃), 3.50-3.57 (m, 1H, —CH₂—O), 3.61 (s, 2H, —CH₂—N), 3.76-3.99 (m, 1H, —CH₂—), 5.34 (t, ³J_{HH}=3.3 Hz, 1H, O—CH—O), 6.96 (d, ³J_{HH}=8.6 Hz, 2H, C₀²H), 7.09-7.26 (m, 2H, C₀²H).

¹³C-{¹H} NMR (75 MHz, CDCl₃) δ (ppm): 18.78 (s, —CH₂—), 25.16 (s, —CH₂—), 30.33 (s, —CH₂—), 35.63 (s, —CH₃), 55.31 (s, —CH₂—N), 61.96 (s, —CH₂—O), 96.38 (s, O—CH—O), 116.36 (s, C₀²), 129.25 (s, C₀³), 132.78 (s, C₀³), 156.12 (s, C₀⁴).

Compound 4b

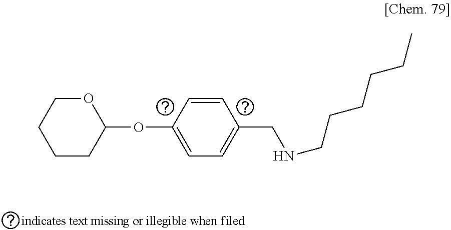 [SMILES: CCCCCCNCc1ccc(OC2CCCCO2)cc1]

1-Aminohexane (1.9 mL, 14.49 mmol) is added to a solution of 4-((tetrahydro-2H-pyran-2-yl)oxy)benzaldehyde (1 g, 4.83 mmol) in THF (30 mL). After stirring overnight at ambient temperature, the mixture is concentrated to dryness under reduced pressure. The residue obtained, Pd/C at 10% (241 mg) and MeOH (40 mL) are introduced into a Fisher-Porter-type tube. The tube is slowly placed under vacuum so as to evacuate the oxygen. The H2 pressure is subsequently adjusted to 5 bar. The reaction mixture is stirred for 2 hours at ambient temperature. After slow depressurization, the reaction mixture is recovered, filtered and concentrated under reduced pressure. The residue is purified by silica gel chromatography (eluent: EtOAc) to obtain compound 4b in the form of a transparent oil at a yield of 70%.

¹H NMR (300 MHz, CDCl₃) δ (ppm): 0.76-1.05 (m, 3H, —CH₃), 1.17-1.43 (m, 3H, —CH₂—), 1.43-1.59 (m, 3H, —CH₂—), 1.59-1.76 (m, 3H, —CH₂—), 1.75-1.95 (m, 3H, —CH₂—), 1.90-2.04 (m, 3H, —CH₂—), 2.61 (t, ³J_{HH}=7.1 Hz, 2H, —CH₂N), 3.56-3.63 (m, 1H, —CH₂—O), 3.72 (s, 2H, C₀⁴—CH₂—), 3.88-3.96 (m, 1H, —CH₂—O), 5.40 (t, ³J_{HH}=3.3 Hz, 1H, O—CH₂—O), 7.01 (d, ³J_{HH}=8.6 Hz, 2H, C₀²H), 7.23 (d, ³J_{HH}=8.6 Hz, 2H, C₀³H).

¹³C-{¹H} NMR (75 MHz, CDCl₃) δ (ppm): 14.04 (s, CH₃—), 18.82 (s, —CH₂—), 22.62 (s, —CH₂—), 25.24 (s, —CH₂—), 27.06 (s, —CH₂—), 30.09 (s, —CH₂—), 30.39 (s, —CH₂—), 31.80 (s, —CH₂—), 49.46 (s, —CH₂—), 53.54 (s, —CH₂N), 61.97 (s, —CH₂—O), 96.41 (s, O—CH—O), 116.36 (s, C₀²), 129.12 (s, C₀³), 133.72 (s, C₀⁴), 156.01 (s, C₀¹).

Compound 5b

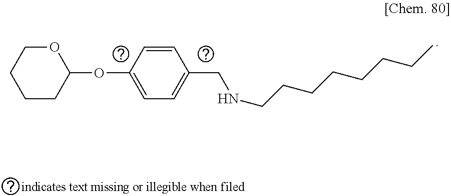 [SMILES: CCCCCCCCNCc1ccc(OC2CCCCO2)cc1]

1-Aminooctane (2.4 mL, 14.49 mmol) is added to a solution of 4-((tetrahydro-2H-pyran-2-yl)oxy)benzaldehyde (1 g, 4.83 mmol) in THF (30 mL). After stirring overnight at ambient temperature, the mixture is concentrated to dryness under reduced pressure. The residue obtained, Pd/C at 10% (241 mg) and MeOH (40 mL) are introduced into a Fisher-Porter-type tube. The tube is slowly placed under vacuum so as to evacuate the oxygen. The H2 pressure is subsequently adjusted to 5 bar. The reaction mixture is stirred for 2 hours at ambient temperature. After slow depressurization, the reaction mixture is recovered, filtered and concentrated under reduced pressure. The residue is purified by silica gel chromatography (eluent: EtOAc) to obtain compound 5b in the form of a transparent oil at a yield of 76%.

¹H NMR (400 MHz, CDCl₃) δ (ppm): 0.90 (t, ³J_{HH}=6.8 Hz, 3H, —CH₃), 1.28-1.34 (m, 10H, —CH₂—), 1.45-1.53 (m, 3H, —CH₂—), 1.54-1.79 (m, 2H, —CH₂—), 1.85-1.90 (m, 2H, —CH₂—), 1.97-2.15 (m, 1H, —CH₂—), 2.62 (t, ³J_{HH}=7.1 Hz, 2H, —CH₂N), 3.59-3.64 (m, 1H, —CH₂—O), 3.74 (s, CH₂—C₀⁴), 3.91-3.97 (m, 1H, —CH₂—O), 5.42 (t, ³J_{HH}=3.4 Hz, 1H, —CH—O), 7.02 (d, ³J_{HH}=8.6 Hz, 2H, C₀²H), 7.24 (d, ³J_{HH}=8.5 Hz, 1H, C₀³H).

¹³C-{¹H} NMR (101 MHz, CDCl₃) δ (ppm): 14.11 (s, CH₃—), 18.85 (s, —CH₂—), 22.67 (s, —CH₂—), 25.24 (s, —CH₂—), 27.40 (s, —CH₂—), 29.28 (s, —CH₂—), 29.55 (s, —CH₂—), 30.12 (s, —CH₂—), 30.41 (s, —CH₂—), 31.84 (s, —CH₂—), 49.47 (s, —CH₂—), 53.56 (s, —CH₂N), 62.04 (s, —CH₂O), 96.45 (s, O—CH—O), 116.39 (s, C₀²), 129.18 (s, C₀²), 133.70 (s, C₀⁴), 156.02 (s, C₀¹).

Compound 3c

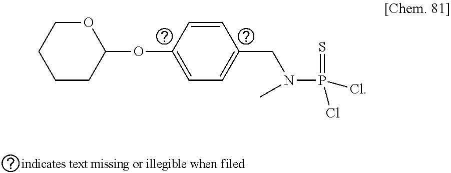 [SMILES: CN(Cc1ccc(OC2CCCCO2)cc1)P(=S)(Cl)Cl]

A solution of dendrimer 3b (1 g, 4.52 mmol) is added dropwise to a solution of PSCl₃ (2.2 mL, 22.6 mmol) in DCM (100 mL) in the presence of Et₃N (0.62 mL, 4.52 mmol) in DCM (40 mL) over a duration of 30 min. After 4 hours of stirring at ambient temperature, the reaction mixture is concentrated to dryness under reduced pressure. The crude residue is subsequently dissolved in 200 mL of DCM then filtered over silica gel to give compound 3c in the form of a transparent oil at a yield of 85%.

³¹P-{¹H} NMR (162 MHz, CDCl₃) δ (ppm): 63.18 (s, PS).

Compound 4c

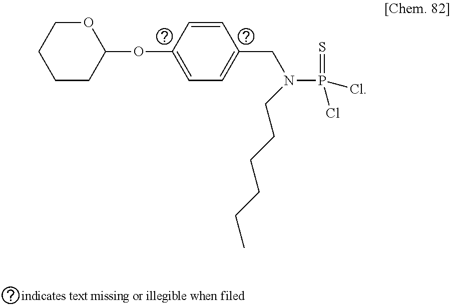 [SMILES: CCCCCCN(Cc1ccc(OC2CCCCO2)cc1)P(=S)(Cl)Cl]

A solution of dendrimer 4b (1 g, 3.43 mmol) is added dropwise to a solution of PSCl₃ (1.73 mL, 17.18 mmol) in DCM (100 mL) brought to −70° C. in the presence of Et₃N (0.47 mL, 3.43 mmol) in DCM (40 mL) over a duration of 30 min. After stirring overnight, the reaction mixture is concentrated to dryness under reduced pressure. The crude residue is subsequently dissolved in 200 mL of DCM then filtered over silica gel to give compound 4c in the form of a transparent oil at a yield of 73%.

³¹P-{¹H} NMR (162 MHz, CDCl₃) δ (ppm): 63.01 (s, PS).

¹H NMR (400 MHz, CDCl₃) δ (ppm): 0.79-0.98 (m, 3H, —CH₃), 1.15-1.33 (m, 6H, —CH₂—), 1.54-1.75 (m, 5H, —CH₂—), 1.82-1.93 (m, 2H, —CH₂—), 1.95-2.06 (m, 1H, —CH₂—), 3.20-3.36 (m, 2H, —CH₂—N), 3.56-3.68 (m, 1H, —CH₂—O), 3.88-3.94 (m, 1H, —CH₂—O), 4.57 (d, ³J_{HP}=16.1 Hz, 2H, C₀⁴—CH₂—), 5.41 (t, ³J_{HH}=3.4 Hz, 1H, O—CH—O), 7.04 (d, ³J_{HH}=8.6 Hz, 2H, C₀²H), 7.25 (d, ³J_{HH}=8.6 Hz, 1H, C₀³H).

Compound 5c

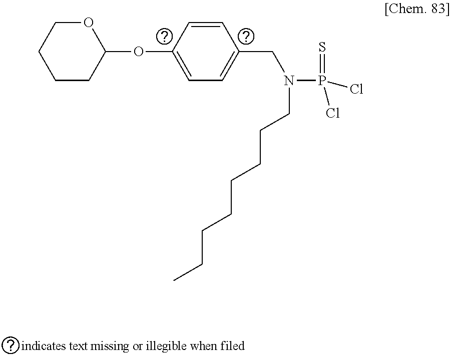 [SMILES: CCCCCCCCN(Cc1ccc(OC2CCCCO2)cc1)P(=S)(Cl)Cl]

A solution of dendrimer 5b (1 g, 3.13 mmol) is added dropwise to a solution of PSCl₃ (1.58 mL, 15.67 mmol) in DCM (100 mL) brought to −70° C. in the presence of Et₃N (0.43 mL, 3.13 mmol) in DCM (40 mL) over a duration of 30 min. After stirring overnight, the reaction mixture is concentrated to dryness under reduced pressure. The crude residue is subsequently dissolved in 200 mL of DCM then filtered over silica gel to give compound 4c in the form of a transparent oil at a yield of 70%.

³¹P-{¹H} NMR (162 MHz, CDCl₃) δ (ppm): 62.92 (s, PS).

¹H NMR (400 MHz, CDCl₃) δ (ppm): 0.90 (t, ³J_{HH}=6.9 Hz, 3H), 1.16-1.42 (m, 1 OH, —CH₂—), 1.52-1.78 (m, 5H, —CH₂—), 1.85-1.96 (m, 2H, —CH₂—), 1.99-2.09 (m, 1H, —CH₂—), 3.21-3.32 (m, 2H, —CH₂—N), 3.57-3.71 (m, 1H, —CH₂-0), 3.90-3.96 (m, 1H, —CH₂—O), 4.59 ³J_{HP}=16.1 Hz, 2H, C₀⁴—CH₂—), 5.43 (t, ³J_{HH}=3.3 Hz, 1H, O—CH₂—O), 7.06 (d, ³J_{HH}=8.6 Hz, 2H, C₀²H), 7.27 (d, ³J_{HH}=8.5 Hz, 2H, C₀²H).

Compound 3d

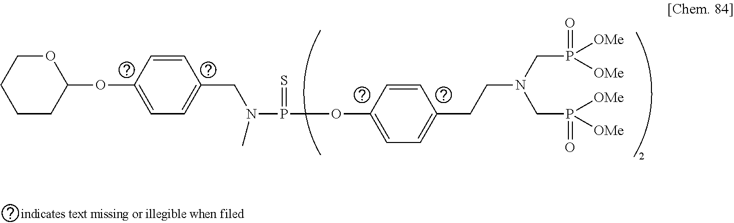

Phenol aminobismethylene phosphonate derived from tyramine (see WO 2005/052031 A1) (2 mmol) is added to a solution of compound 3c (1 mmol) in THF (30 mL) with Cs₂CO₃ (4 mmol). After stirring overnight at ambient temperature, the mixture is centrifuged, filtered then concentrated under reduced pressure. The crude residue is subsequently purified by silica gel chromatographic column with an eluent mixture (MeOH/EtOAc) to give, after elimination of the solvents under reduced pressure, compound 3d in the form of a transparent oil at a yield of 85%.

³¹P-{¹H} NMR (162 MHz, CDCl₃) δ (ppm): 26.95 (s, POMe), 68.48 (s, P═N).

¹H NMR (400 MHz, CDCl₃) δ (ppm): 1.58-1.72 (m, 3H, —CH₂), 1.81-1.94 (m, 2H, —CH₂—), 1.96-2.05 (m, 1H, —CH₂—), 2.78-2.83 (m, 7H, C₁⁴—CH₂, CH₃N—), 3.09 (t, ³J_{HH}=7.6 Hz, 4H, —CH₂N—), 3.22 (d, ³J_{HH}=8.9 Hz, 8H,), 3.55-3.67 (m, 1H, CH₂—O), 3.75 (d, ³J_{HP}=10.3 Hz, 24H, POMe), 3.89-3.95 (m, 1H, CH₂-0), 4.44 (d, ³J_{HP}=12.0 Hz, 2H), 5.41 (t, ³J_{HH}=3.3 Hz, 1H, O—CH₂—O), 6.99 (d, ³J_{HH}=8.6 Hz, 2H, C₀²H), 7.12 (d, ³J_{HH}=8.4, 4H, C₁²H), 7.16-7.25 (m, 6H, C₀³H, C₁³H).

Compound 4d

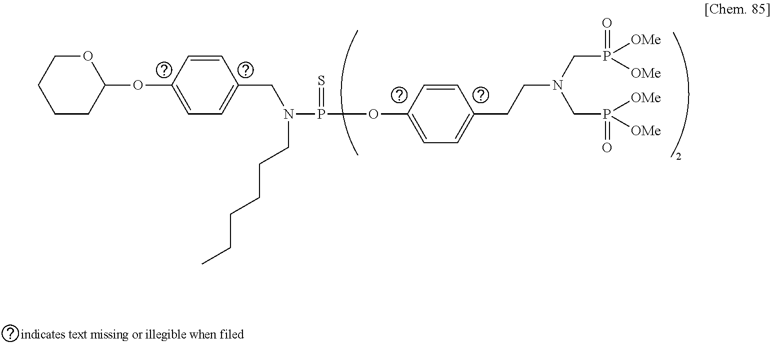

Phenol aminobismethylene phosphonate derived from tyramine (see WO 2005/052031 A1) (2 mmol) is added to a solution of compound 4c (1 mmol) in THF (30 mL) with Cs₂CO₃ (4 mmol). After stirring overnight at ambient temperature, the mixture is centrifuged, filtered then concentrated under reduced pressure. The crude residue is subsequently purified by silica gel chromatographic column with an eluent mixture (MeOH/EtOAc) to give, after elimination of the solvents under reduced pressure, compound 4d in the form of a transparent oil at a yield of 81%.

³¹P-{¹H} NMR (121 MHz, CDCl₃) δ (ppm): 26.60 (s, POMe), 67.70 (s, PS).

Compound 5d

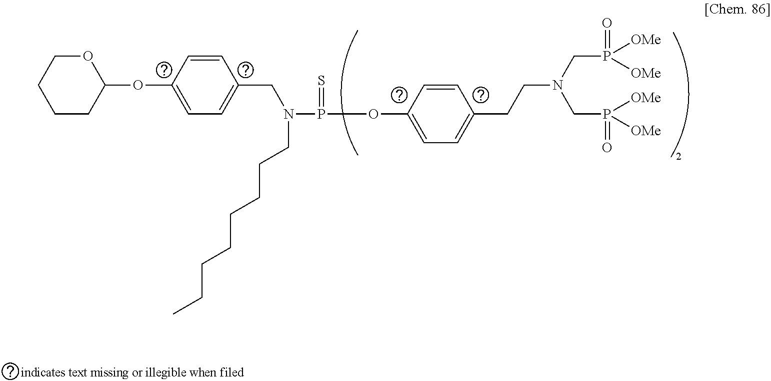

Phenol aminobismethylene phosphonate derived from tyramine (see WO 2005/052031 A1) (2 mmol) is added to a solution of compound 5c (1 mmol) in THF (30 mL) with Cs₂CO₃ (4 mmol). After stirring overnight at ambient temperature, the mixture is centrifuged, filtered then concentrated under reduced pressure. The crude residue is subsequently purified by silica gel chromatographic column with an eluent mixture (MeOH/EtOAc) to give, after elimination of the solvents under reduced pressure, compound 5d in the form of a transparent oil at a yield of 75%.

³¹P-{¹H} NMR (121 MHz, CDCl₃) δ (ppm): 26.92 (s, POMe), 68.02 (s, PS).

Compound 3e

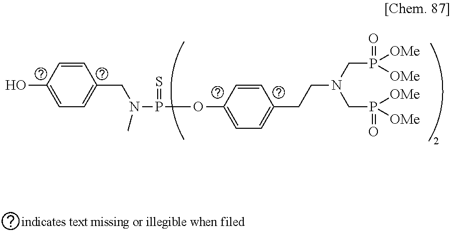

Pyridinium p-toluenesulfonate (1.5 mmol) is added to a solution of compound 3d (1 mmol) in MeOH (30 mL). The reaction mixture is stirred overnight at ambient temperature. The solvent is eliminated under reduced pressure and the residue is purified by silica gel chromatography (eluent: MeOH/AcOEt) to give, after elimination of the solvents under reduced pressure, compound 3e in the form of a transparent oil at a yield of 80%.

³¹P-{¹H} NMR (162 MHz, CDCl₃) δ (ppm): 26.76 (s, POMe), 68.38 (s, PS).

Compound 4e

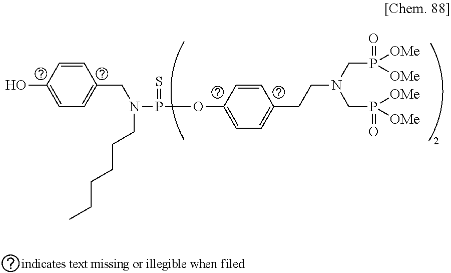

Pyridinium p-toluenesulfonate (1.5 mmol) is added to a solution of compound 4d (1 mmol) in MeOH (30 mL). The reaction mixture is stirred overnight at ambient temperature. The solvent is eliminated under reduced pressure and the residue is purified by silica gel chromatography (eluent: MeOH/EtOAc) to give, after elimination of the solvents under reduced pressure, compound 3e in the form of a transparent oil at a yield of 78%.

³¹P-{¹H} NMR (162 MHz, CDCl₃) δ (ppm): 26.70 (s, POMe), 68.2 (s, PS).

¹H NMR (300 MHz, CDCl₃) δ (ppm): 0.74-0.91 (m, 3H, CH₃—), 1.11-1.39 (m, 6H, —CH₂—), 1.58 (br s, 2H, —CH₂—), 2.75 (t, ³J_{HH}=7.8 Hz, 4H, C₁⁴—CH₂—), 2.91-3.17 (m, 6H, —CH₂N—, —CH₂N—), 3.74 (d, ³J_{HP}=10.6, 12H, POMe), 3.75 (d, ³J_{HP}=10.6, 12H, POMe), 4.38 (d, ³J_{HH}=14.1 Hz, 2H, C₀⁴—CH₂—), 6.61 (d, ³J_{HH}=8.5 Hz, 2H, C₀²H), 6.70 (d, ³J_{HH}=8.6 Hz, 2H, C₀³H), 6.91-7.21 (m, 8H, C₁²H, C₁³H), 8.80 (s, 1H, OH).

¹³C-{¹H} NMR (101 MHz, CDCl₃) δ (ppm): 13.99 (s, CH₃—), 22.57 ((s, —CH₂—), 26.57 (s, CH₂—), 27.00 (s, CH₂—), 31.50 (s, CH₂—), 33.20 (s, CH₂—), 44.66 (br s, CH₂—NP), 48.96 (d, ²J_{CP}=8.3 Hz, C₀⁴—CH₂—), 49.51 (dd, ¹J_{CP}=157.7, ²J_{CP}=7.3 Hz, —CH₂—P), 52.78 (m, POCH₃), 58.38 (t, ³J_{CP}=7.4 Hz, —CH₂—N), 114.87 (s, C₀²), 121.33 (d, ³J_{CP}=4.8 Hz, C₁²), 127.57 (d, ³J_{CP}=3.5 Hz, C₀⁴), 129.64 (s, C₁³), 129.76 (s, C₀³), 135.73 (s, C₁⁴), 149.33 (d, ²J_{HP}=7.2 Hz, C₁¹), 156.70 (s, C₀¹).

Compound 5e

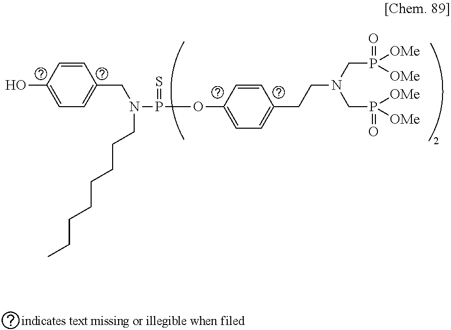

Pyridinium p-toluenesulfonate (1.5 mmol) is added to a solution of compound 5d (1 mmol) in MeOH (30 mL). The reaction mixture is stirred overnight at ambient temperature. The solvent is eliminated under reduced pressure and the residue is purified by silica gel chromatography (eluent: MeOH/EtOAc) to give, after elimination of the solvents under reduced pressure, compound 5e in the form of a transparent oil at a yield of 82%.

³¹P-{¹H} NMR (121 MHz, CDCl₃) δ (ppm): 26.72 (s, POMe), 68.41 (s, PS).

### Compounds 3-G′₁(OMe) from 3e

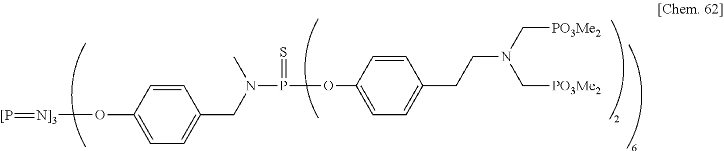

Hexachlorocyclotriphosphazene (60 mg, 0.17 mmol) is added to a solution of compound 3e (1 g, 1.04 mmol) in THF (30 mL) with Cs₂CO₃ (794 mg, 2.08 mmol). After stirring overnight at 45° C., the mixture is centrifuged, filtered then concentrated under reduced pressure. Dendrimer 3-G′₁ was obtained in the form of a transparent oil at a yield of 90%.

³¹P-{¹H} NMR (243 MHz, CDCl₃) δ (ppm): 8.40 (s, N═P), 26.85 (s, PO), 68.32 (s, PS);

¹H NMR (600 MHz, CDCl₃) δ (ppm): 2.75 (d, ³J_{HH}=10.1 Hz, 24H, C₁⁴—CH₂), 2.77 (d, ³J_{HP}=7.6 Hz, 18H, CH₃), 3.06 (d, ³J_{HH}=7.7 Hz, 24H, C₁⁴—CH₂—CH₂), 3.19 (d, ²J_{HP}=9.4 Hz, 48H, CH₂—P), 3.73 (d, ³J_{HP}=10.6 Hz, 144H, POMe), 6.94 (d, ³J_{HH}=8.2 Hz, 12H, C₀²H), 7.07 (d, ³J_{HH}=8.1 Hz, 24H, C₁²H), 7.18 (br d, ³J_{HH}=8.2 Hz, 36H, C₀³ and C₁³H);

¹³C-{¹H} NMR (151 MHz, CDCl₃) δ (ppm): 33.05 (s, CH₂), 33.64 s, (CH₃N), 49.46 (dd, ¹J_{CP}=157.3, ³J_{CP}=7.2 Hz, CH₂—P), 53.56-51.75 (m, POCH₃), 53.47 (d, ²J_{CP}=10.3 Hz, C₁⁴—CH₂), 58.22 (t, ³J_{CP}=7.7 Hz, C₁⁴—CH₂—CH₂), 120.91 (br d, ³J_{CP}=4.4 Hz, C₀² and C₁²), 129.30 (s, C₀³), 129.87 (s, C₁³), 134.10 (br d, ²J_{CP}=4.5 Hz, C₀⁴), 136.22 (s, C₁⁴), 149.35 (d, ²J_{CP}=7.5 Hz, C₁¹), 150.01 (br s, C₀¹).

### Compounds 4-G′₁(OMe) from 4e

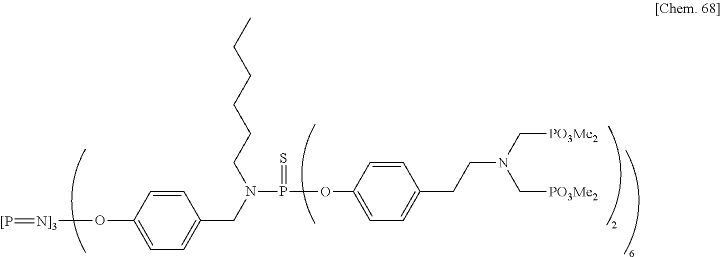

Hexachlorocyclotriphosphazene (56 mg, 0.16 mmol) is added to a solution of compound 4e (1 g, 0.97 mmol) in THF (30 mL) with Cs₂CO₃ (632 mg, 1.94 mmol). After stirring overnight at 45° C., the mixture is centrifuged, filtered then concentrated under reduced pressure. Dendrimer 4-G′₁ was obtained in the form of a transparent oil at a yield of 85%.

³¹P-{¹H} NMR (162 MHz, CDCl₃) δ (ppm): 8.23 (s, P═N), 26.89 (s, POMe), 68.00 (s, PS).

¹H NMR (400 MHz, CDCl₃) δ (ppm): 0.78 (t, ³J_{HH}=6.6 Hz, 18H, CH₃—), 1.10-1.26 (m, 36H, —CH₂—), 1.41-1.47 (m, 12H, —CH₂—), 2.75 (t, ³J_{HH}=7.6 Hz, 24H, —CH₂-C₁⁴), 3.04 (t, ³J_{HH}=7.7 Hz, 24H, —CH₂—N), 3.09-3.15 (m, 12H, —CH₂—NP), 3.19 (d, ²J_{HP}=9.3 Hz, 18H, —CH₂—P), 3.65-3.79 (m, 144H, POMe), 4.53 (d, ³J_{HP}=13.1 Hz, 12H, —CH₂-C₀⁴), 6.95 (d, ³J_{HH}=8.1 Hz, 12H, C₀²H), 7.06 (d, ³J_{HH}=8.2 Hz, 24H, C₁²H), 7.16 (d, ³J_{HH}=8.2 Hz, 24H, C₁²H), 7.22 (d, ³J_{HH}=8.3 Hz, 24H, C₀³H).

### Compounds 5-G′₁ (OMe) from 5e

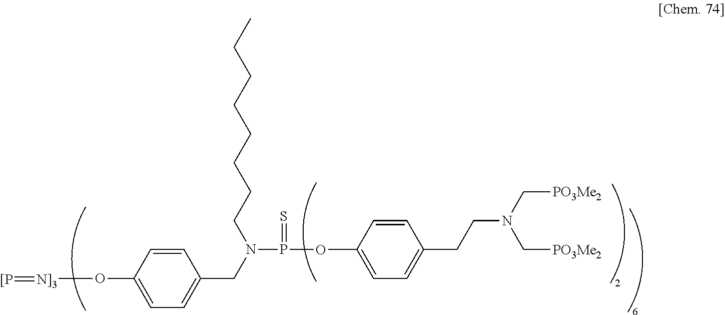

Hexachlorocyclotriphosphazene (54 mg, 0.15 mmol) is added to a solution of compound 5e (1 g, 0.94 mmol) in THF (30 mL) with Cs₂CO₃ (616 mg, 1.9 mmol). After stirring overnight at 45° C., the mixture is centrifuged, filtered then concentrated under reduced pressure. Dendrimer 5-G′₁ was obtained in the form of a transparent oil at a yield of 88%.

³¹P-{¹H} NMR (162 MHz, CDCl₃) δ (ppm): 8.22 (s, P═N), 26.89 (s, POMe), 68.01 (s, PS).

¹H NMR (400 MHz, CDCl₃) δ (ppm): 0.83 (t, ³J_{HH}=7.0 Hz, 18H, CH₃), 1.01-1.41 (m, 60H, CH₂), 1.45 (br s, 12H, CH₂), 2.77 (t, ³J_{HH}=7.5 Hz, 24H, C₁⁴—CH₂—), 3.06 (t, ³J_{HH}=7.7 Hz, 24H, CH₂N), 3.09-3.15 (m, 12H, CH₂NPS), 3.20 (d, ²J_{HP}=9.3 Hz, 48H, CH₂P), 3.74 (d, ³J_{HP}=10.6, 1.5 Hz, 72H, POMe), 3.75 (d, ³J_{HP}=10.6, 1.5 Hz, 72H, POMe), 4.55 (d, ³J_{HP}=13.1 Hz, 12H, C₀⁴—CH₂—), 6.96 (d, ³J_{HH}=8.2 Hz, 12H, C₀²H), 7.02-7.12 (m, 24H, C₁²H), 7.18 (d, ³J_{HH}=8.5 Hz, 24H, C₁³H), 7.24 (d, ³J_{HH}=8.4 Hz, 12H, C₀³H).

¹³C-{¹H} NMR (101 MHz, CDCl₃) δ (ppm): 14.11 (s, CH₃), 22.61 (s, CH₂), 26.81 (s, CH₂), 27.62 (s, CH₂), 29.18 (s, CH₂), 31.72 (s, CH₂), 33.07 (s C₁⁴—CH₂—), 45.74 (br s, CH₂NS), 49.45 (dd, ¹J_{CP}=157.2, ³J_{CP}=7.1 Hz, CH₂P), 49.46 (br s, C₀⁴—CH₂—), 52.59-52.71 (m, POMe), 58.30 (t, ³J_{CP}=7.7 Hz, CH₂N), 120.90 (s, C₀²H), 121.02 (d, ²J_{CP}=4.7 Hz, C₀²H), 129.39 (s, C₀³H), 129.77 (s, C₁³H), 134.28 (s, C₀⁴), 136.09 (s, C₁⁴), 149.54 (d, ²J_{CP}=7.8 Hz, C₁¹), 150.00 (d, ²J_{CP}=4.5 Hz, C₀¹).

Semi-Convergent Synthesis of Bifunctional 1st Generation Dendrimers

Scheme 5 describes the reaction scheme for semi-convergent synthesis of bifunctional 1 st generation dendrimers:

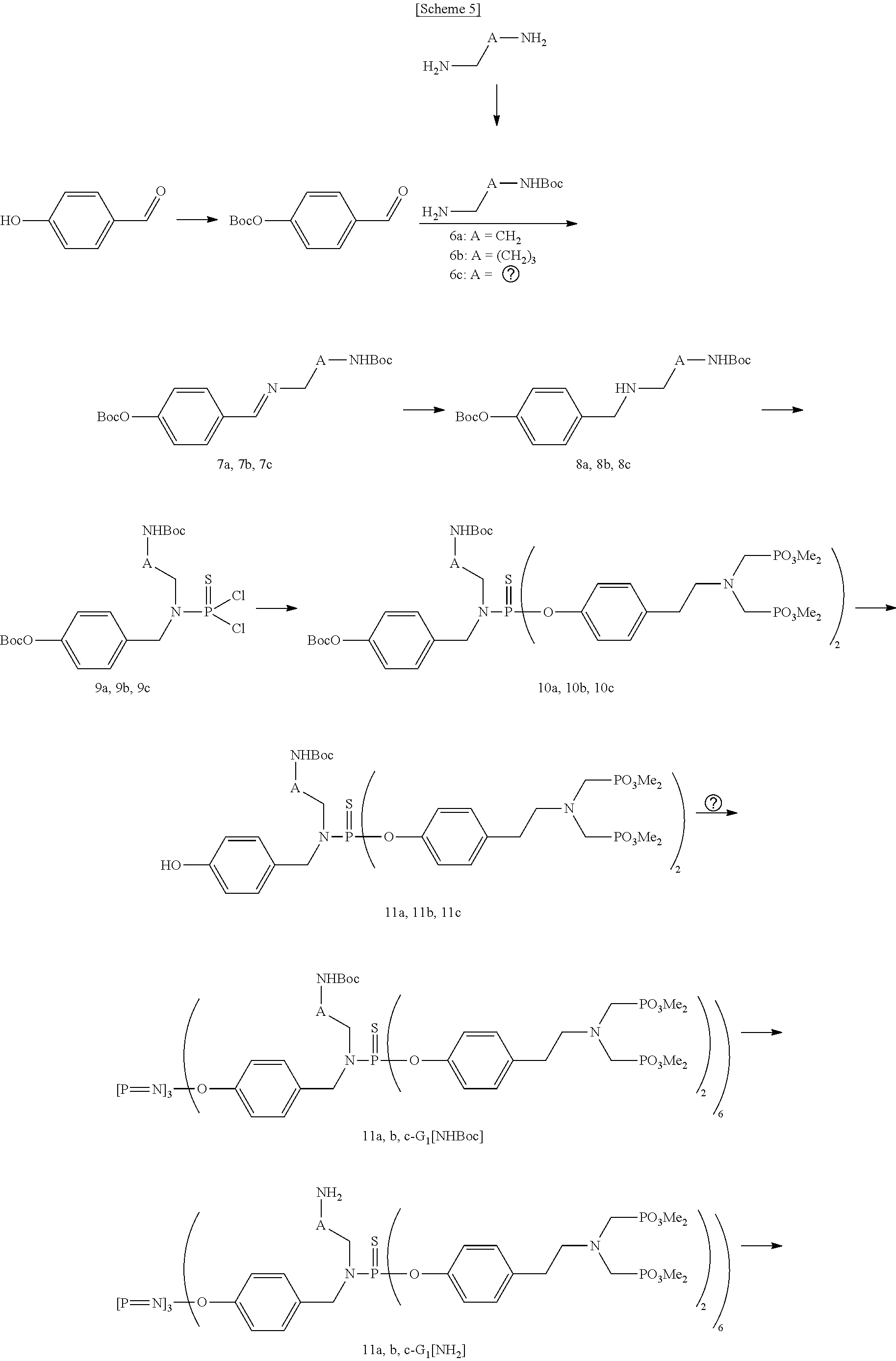

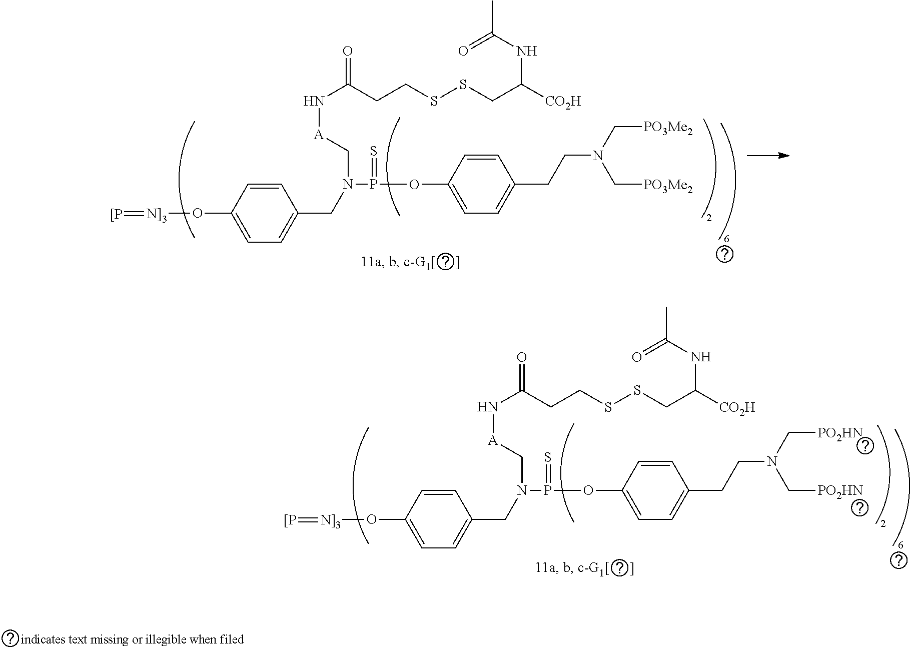

### Synthesis of Compounds 7a, 7b and 7c

Compound 6a, b or c (5.25 mmol) is added to a solution of tert-butyl (4-formylphenyl)carbonate (5 mmol) in DCM (50 mL). After stirring overnight at ambient temperature, the reaction is terminated. Compound 7a, b or c is not isolated and is directly used thereafter.

### Characterization of Compound 7a

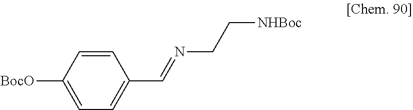

¹H RMN (400 MHz, CD₃CN) δ (ppm) 1.39 (s, 9H Boc-NH), 1.55 (s, 9H Boc-O), 3.34 (q, ³J_{HH}=6.1 Hz, 2H, CH₂NHBoc), 3.65 (m, 2H, CH═N—CH₂), 5.42 (s, NHBoc) 7.25 (d, ³J_{HH}=8.6 Hz, 2H, C₀²H), 7.80 (d, ³J_{HH}=8.6 Hz, 2H, C₀³H), 8.30 (s, 1H, CH═N).

### Characterization of Compound 7b

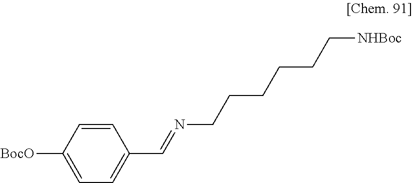

¹H RMN (400 MHz, CD₂Cl₂) δ (ppm) 1.45 (s, 9H, Boc-NH), 1.57 (s, 9H, Boc-OPh), 3.21 (q, ³J_{HH}=6.3 Hz, 2H, CH₂NHBoc), 3.47-3.75 (m, 2H, CH═N—CH₂), 7.23 (d, ³J_{HH}=8.7 Hz, 2H, C₀²H), 7.78 (d, ³J_{HH}=8.6 Hz, 2H, C₀³H), 8.32 (s, 1H, HC═N).

### Characterization of Compound 7c

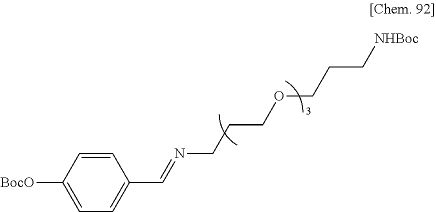

Compound 7c is used directly in the next step.

### Synthesis of Compounds 8a, 8b and 8c

Pd/C 10% (250 mg) is added to a solution of compound 7a,b,c (5 mmol) in anhydrous ethanol (50 mL) under 5 bar of hydrogen. After 1.5 hours of stirring at ambient temperature, the reaction is terminated. Compound 8a,b,c is purified over silica gel with a DCM/MeOH gradient at a yield of 70% to 80%.

### Characterization of Compound 8a

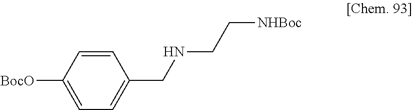

¹H RMN (400 MHz, CDCl₃) δ (ppm) 1.45 (s, 4H), 1.56 (s, 2H), 2.74 (t, J=5.8 Hz, 1H), 3.19-3.27 (m, 3H), 3.77 (s, 1H), 5.01 (s, 1H), 7.12 (d, ³J_{HH}=9.5 Hz, OH), 7.31 (d, ³J_{HH}=8.3 Hz, 1H).

¹³C-{¹H} NMR (101 MHz, CDCl₃) δ (ppm) 27.69 (s, Boc-O-Ph), 28.41 (s, Boc-NH), 48.45 (s, N₀CH₂CH₂), 52.82 (s, CH₂CH₂NHBoc), 79.33 (brs, NHCOC(CH₃)₃), 83.50 (s, OCOC(CH₃)₃), 121.21 (s, C₀²), 129.03 (s, C₀³), 137.58 (s, C₀⁴), 150.05 (s, C₀¹), 151.96 (s, OC(O)COC(CH₃)₃), 156.19 (s, NHC(O)COC(CH₃)₃).

### Characterization of Compound 8b

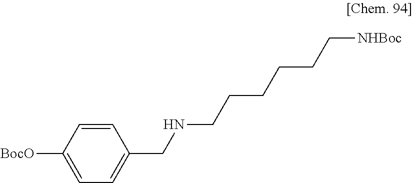

¹H NMR (300 MHz, CD₂Cl₂) δ (ppm): 1.51 (m, 26H, CH₂CH₂CH₂CH₂, Boc-O, Boc-NH), 2.63 (t, J=7.1 Hz, 2H, N₀CH₂), 3.09 (q, J=6.8 Hz, 2H, CH₂NHBoc), 3.80 (s, 2H, C₀⁴CH₂), 4.83 (s, 1H, NHBoc), 7.12 (d, ³J_{HH}=8.5 Hz, 2H, C₀²H), 7.37 (d, ³J_{HH}=8.5 Hz, 2H, C₀³H).

### Characterization of Compound 8c

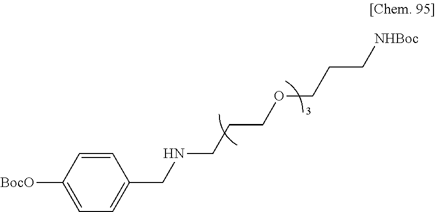

¹H NMR (300 MHz, CDCl₃) δ (ppm): 1.43 (s, 9H, NHBoc), 1.55 (s, 9H, NHBoc), 1.65-1.92 (m, 4H, N₀CH₂CH₂, CH₂CH₂NHBoc), 2.45 (s, NH), 2.73 (t, ³J_{HH}=6.8 Hz, 2H, N₀CH2), 3.21 (q, ³J_{HH}=5.3 Hz, 2H, CH₂NHBoc), 3.38-3.70 (m, 12H, CH₂O), 3.78 (s, 2H, C₀⁴CH₂), 5.13 (s, 1H, NHBoc), 7.11 (d, ³J_{HH}=8.5 Hz, 2H, C₀²H), 7.33 (d, ³J_{HH}=8.1 Hz, 2H, C₀³H).

### Synthesis of Compounds 9a, 9b and 9c

A solution of compound 8a,b,c (4 mmol) with Et₃N (6 mmol) in CH₂Cl₂ (30 mL) is added dropwise to a solution of PSCl₃ (12 mmol) in CH₂Cl₂ (100 mL) with molecular sieve over a duration of 30 min. After 2 h to 12 h of stirring at ambient temperature, the mixture is concentrated to dryness under reduced pressure. The crude residue is subsequently purified over silica gel with a 100→98/2 DCM/THF gradient, to give compound 9a,b,c in the form of a transparent oil at a yield of 70 to 85%.

### Characterization of Compound 9a

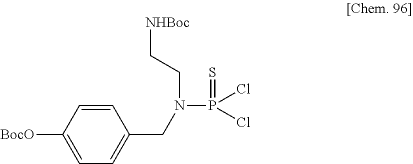

³¹P-{¹H} NMR (162 MHz, CDCl₃) δ (ppm): 64.17.

¹H NMR (400 MHz, CDCl₃) δ (ppm): 1.46 (s), 1.58 (s), 3.20-3.61 (m), 4.68 (d, J=15.3 Hz), 7.20 (d, J=8.5 Hz), 7.41 (d, J=8.1 Hz).

¹³C-{¹H} NMR (101 MHz, CDCl₃) δ (ppm): 27.69, 28.40, 37.42, 46.47, 50.11, 79.77, 83.82, 121.78, 129.35, 132.41 (d, J=5.6 Hz), 150.96, 151.74, 155.80.

### Characterization of Compound 9b

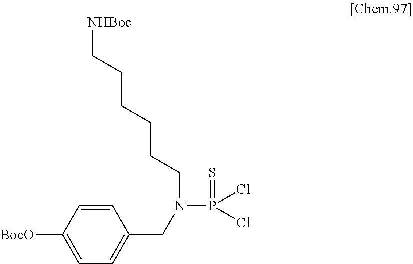

¹H NMR (400 MHz, CD₂Cl₂) δ (ppm): 1.23-1.38 (m), 1.45 (s), 1.57 (s), 1.60-1.78 (m), 3.08 (q, J=6.6 Hz), 3.20-3.43 (m), 4.68 (d, J=16.6 Hz), 7.20 (d, J=8.5 Hz), 7.41 (d, J=8.5 Hz).

³¹P-{¹H} NMR (162 MHz, CD₂Cl₁₂) δ (ppm): 63.16.

¹³C-{¹H} NMR (101 MHz, CD₂Cl₂) δ (ppm): 26.25 (d, J=4.5 Hz), 26.85 (d, J=3.1 Hz), 27.41, 28.14, 29.89, 40.32, 47.40 (d, J=2.4 Hz), 50.21 (d, J=4.5 Hz), 78.56, 83.49, 121.66, 129.05, 133.15 (d, J=5.5 Hz), 150.89, 151.69, 155.76.

### Characterization of Compound 9c

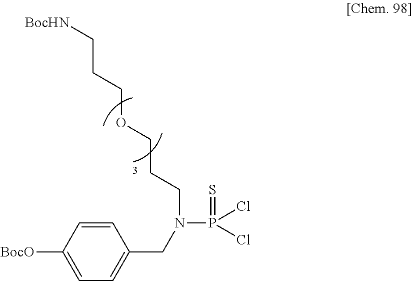

¹H NMR (300 MHz, CDCl₃) δ (ppm): 1.45 (s), 1.58 (s), 1.76 (p, J=6.2 Hz), 1.85-1.99 (m), 3.10-3.29 (m), 3.31-3.76 (m), 4.66 (d, J=16.5 Hz), 4.97 (s), 7.19 (d, J=8.6 Hz), 7.38 (d, J=8.5 Hz).

³¹P-{¹H} NMR (121 MHz, CDCl₃) δ (ppm): 63.07.

### Synthesis of Compounds 10a, 10b and 10c

Cs₂CO₃ (16.4 mmol) and phenol aminobismethylene phosphonate derived from tyramine (8.2 mmol) are added in succession to a solution of compound 9a,b,c (4 mmol) in THF (30 mL). After stirring overnight at ambient temperature, the mixture is centrifuged, filtered then concentrated under reduced pressure. The product is subsequently purified by a silica gel chromatographic column (2.5/97.5 to 5/95 MeOH/DCM eluent gradient), to give compound 10a,b,c in the form of a white powder at a yield of 60 to 80%.

### Characterization of Compound 10a

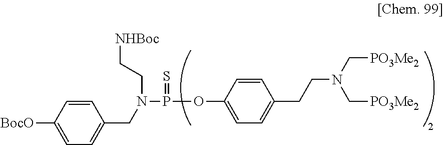

³¹P-{¹H} NMR (162 MHz, C₆D₆) δ (ppm): 26.67 (s, POMe), 68.58 (s, PS).

### Characterization of Compound 10b

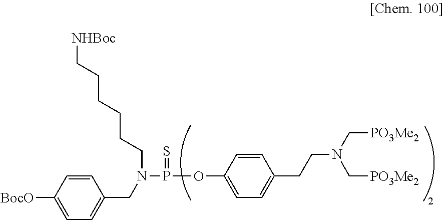

³¹P-{¹H} NMR (121 MHz, CDCl₃) δ (ppm): 26.70 (s, POMe), 68.65 (s, PS).

### Characterization of Compound 10c

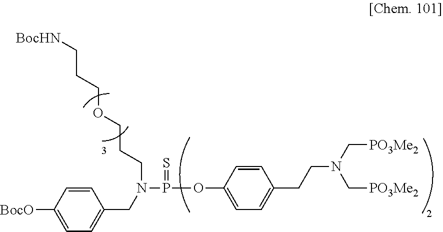

³¹P-{¹H} NMR (162 MHz, CDCl₃) δ (ppm): 26.68 (s, POMe), 68.80 (s, PS).

### Synthesis of Compounds 11a, 11b and 11c

An excess of methylamine in EtOH (8 M) is added to a solution of compound 10 (mmol) in MeOH (10 mL). After stirring overnight at ambient temperature, the mixture is centrifuged, filtered then concentrated under reduced pressure.

The product is subsequently purified by a silica gel chromatographic column (2.5/97.5 to 5/95 MeOH/DCM eluent gradient), to give compound 11 in the form of a transparent oil at a yield of greater than 80%.

### Characterization of Compound 11a

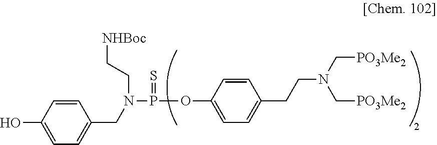

³¹P-{¹H} NMR (121 MHz, CDCl₃) δ (ppm): 26.68, 68.75.

### Characterization of Compound 11b

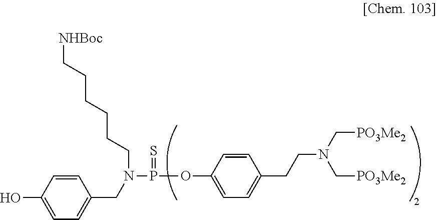

³¹P-{¹H} NMR (121 MHz, 00013) δ (ppm): 26.68, 68.75.

### Characterization of Compound 11c

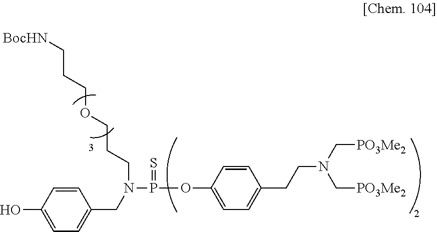

³¹P-{¹H} NMR (121 MHz, CDCl₃) δ (ppm): 26.71, 68.80.

### Synthesis of compounds 11a-G1(NHBoc), 11b-G1(NHBoc and 11c-G1(NHBoc)

Compound 11a,b,c (0.72 mmol) and cesium carbonate (1.45 mmol) are added to a solution of N₃P₃Cl₆ (0.1 mmol) in THF. After stirring overnight at a temperature of 40° C., the mixture is centrifuged, filtered and concentrated under reduced pressure. The product is subsequently purified using a silica gel chromatography column to give compound 11a,b,c-G₁(NHBoc) in the form of a transparent oil. The yield is 60 to 80%.

### Characterization of Compound 11a-G₁(NHBoc)

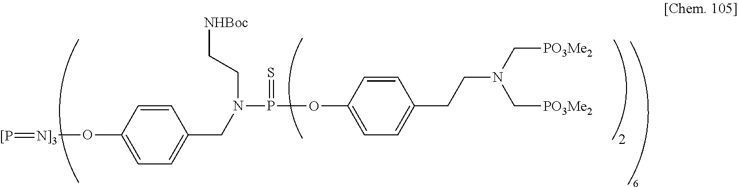

³¹P-{¹H} NMR (162 MHz, CDCl₃) δ (ppm): 8.08 (s, P₀), 26.88 (s, POMe), 68.56 (s, P₁).

¹H NMR (400 MHz, CDCl₃) δ (ppm): 1.37 (s, 54H, NHBoc), 2.66-2.88 (m, C₁⁴CH₂, 24H), 3.00-3.11 (m, C₁⁴CH₂ CH₂, 24H), 3.21 (m, 60H, CH₂POMe, CH₂NHBoc), 3.28-3.37 (m, N₀CH2), 3.75 (d, ³J_{HP}=10.6 Hz, 144H, POMe), 4.58 (d, ³J_{HP}=12.1 Hz, 12H, C₀⁴CH₂), 5.09 (s, 6H, NHBoc), 6.97 (d, ³J_{HH}=8.2 Hz, 12H, C₀²), 7.08 (d, ³J_{HH}=7.1 Hz, 24H, C₁²), 7.18 (d, ³J_{HH}=8.4 Hz, 24H, C₁³), 7.26 (d, ³J_{HH}=8.4 Hz, 12H, C₀³).

¹³C-{¹H} NMR (101 MHz, CDCl₃) δ (ppm): 28.41 (s, Boc), 33.06 (s, C₁⁴CH₂) 38,23 (s, NCH₂CH₂), 45,25 (s, NCH₂CH₂), 48.66 (dd, ¹J_{CP}=157.2, ³J_{CP}=7.1 Hz, CH₂POMe), 49.57 (brs, C₀⁴CH₂), 52.29-53.00 (m, POMe), 58.27 (t, ³J_{CP}=7.7 Hz, C₁⁴CH₂CH₂), 79.11 (s, OC(CH₃)₃), 120.96 (s, C₀²), 121.14 (d, ³J_{CP}=4.5 Hz, C₁²), 129.69 (s, C₀³), 129.83 (s, C₁³), 133.89 (s, C₀⁴), 136.31 (s, C₁⁴), 149.37 (d, ²J_{CP}=7.9 Hz, C₁¹), 150.19 (s, C₀¹), 155.86 (s, C═O).

### Characterization of Compound 11b-G₁ (NHBoc)

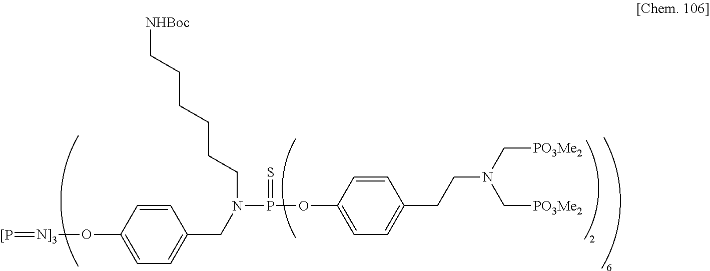

³¹P-{¹H} NMR (162 MHz, MeOD) δ (ppm): 8.92, 27.42, 68.63 ppm.

### Characterization of Compound 11c-G₁(NHBoc)

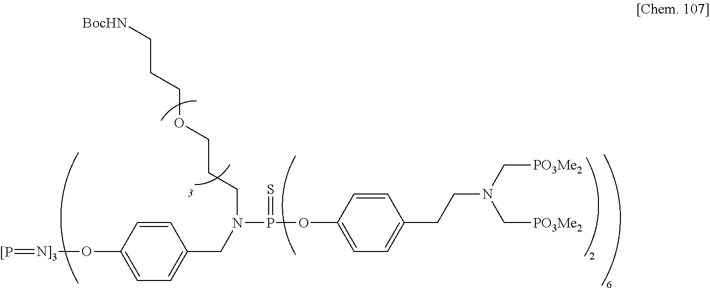

³¹P-{¹H} NMR (162 MHz, CDCl₃) δ (ppm): 8.11 (s, P₀), 26.87 (s, POMe), 68.15 (s, P₁).

¹H NMR (400 MHz, CDCl₃) δ (ppm): 1.41 (s, 54H, NHBoc), 1.59-1.82 (m, 24H, CH₂CH₂CH₂), 2.77 (m, C₁⁴CH₂, 24H), 3.05 (t, ³J_{HH}=7.7 Hz, 24H, C₁⁴CH₂ CH₂), 3.12-3.38 (m, 84H, NCH₂POMe, CH₂NHBoc, N₀CH₂), 3.41-3.64 (m, 60H, CH₂O), 3.74 (³J_{HP}=10.6 Hz, 144H, POMe), 4.55 (d, ³J_{HP}=12.1 Hz, 12H, C₀⁴CH₂), 5.15 (s, NHBoc) 6.95 (d³J_{HH}=8.2 Hz, 12H, C₀²), 7.06 (d, ³J_{HH} 7.1 Hz, 24H, C₁²), 7.17 (d, ³J_{HH}=8.4 Hz, 24H, C₁³), 7.24 (d, ³J_{HH}=8.4 Hz, 12H, C₀³).

¹³C-{¹H} NMR (101 MHz, CDCl₃) δ (ppm): 27.98 (BocHNCH₂CH₂CH₂), 28.45 (s, Boc), 29.66 (N₀CH₂CH₂), 33.03 (s, C₁⁴CH₂), 38.42 (s, N₀CH₂CH₂), 43.05 (s, CH₂NHBoc), 49.44 (¹J_{CP}=157.4, ³J_{CP}=7.1 Hz, CH₂POMe), 49.65 (brs, C₀⁴CH₂), 52.33-52.97 (m, POMe), 58.30 (t, ³J_{CP}=7.7 Hz, C₁⁴CH₂CH₂), 68.49 (s, CH₂O), 69.43 (s, CH₂O), 70.08 (s, CH₂O) 70.12 (s, CH₂O), 70.44 (s, CH₂O), 70.49 (s, CH₂O), 78.75 (s, OC(CH₃)₃), 120.88 (s, C₀²), 121.07 (d, ³J_{CP}=4.7 Hz, C₁²), 129.44 (s, C₀³), 129.80 (s, C₁³), 134.24 (s, C₀⁴), 136.16 (s, C₁⁴), 149.49 (d, ²J_{CP}=7.9 Hz, C₁¹), 150.01 (s, C₀¹), 156.04 (s, C═O).

### Synthesis of Compounds 11a-G₁(NH₂), 11b-G₁(NH₂) and 11b-G₁(NH₂)

Trifluoroacetic acid (5 mL) is added to a solution of compound 11a,b,c-G₁(NHBoc) in DCM (5 mL). After 10 min of stirring, the mixture is evaporated to dryness then co-evaporated 5 times with 3 mL of DCM to give compound 11a,b,c-G₁(NH₂) in the form of the trifluoroacetate salt, in the form of a transparent oil. The yield is greater than 90%.

### Characterization of Compound 11a-G₁(NH₂)

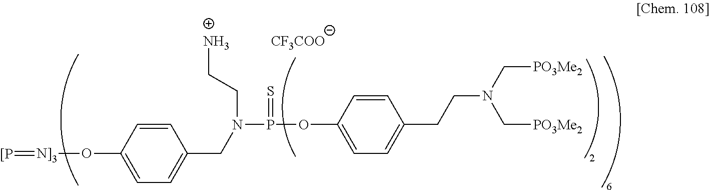

³¹P-{¹H} NMR (243 MHz, CD₃CN) δ (ppm): 8.74 (s, P₀), 26.00 (s, POMe), 68.65 (s, P₁).

¹H NMR (600 MHz, CD₃CN) δ (ppm): 2.81 (t, ³J_{HH}=7.6 Hz, 24H, C₁⁴CH₂), 2.92 (m, 12H, N₀CH2), 3.05 (t, ³J_{HH}=7.7 Hz, 24H, C₁⁴CH₂ CH₂), 3.26 (d, ²J_{HP}=10.4 Hz, 48H, CH₂POMe), 3.49-3.56 (m, 12H, PNCH₂CH₂NH₃⁺), 3.73 (dd, ²J_{HP}=10.7, J=1.5 Hz, 144H, POMe), 4.55 (d, ³J_{HP}=12.8 Hz, 12H, C₀⁴CH₂), 6.91 (d, ³J_{HH}=8.3 Hz, 12H, C₀²), 7.11 (d, ³J_{HH}=8.0 Hz, 24H, C₁²), 7.17-7.28 (m, 36H, C₁³, C₀⁰).

¹³C-{¹H} NMR (151 MHz, CD₃CN) δ (ppm): 31.38 (s, C₁⁴CH₂), 37.34 (s, NCH₂CH₂), 42.63 (s, NCH₂CH₂), 48.66 (dd, ¹J_{CP}=157.2 Hz, ³J_{CP}=7.1 Hz, CH₂POMe), 49.60 (brs, C₀⁴CH₂), 53.01 (d, ²J_{CP}=7.2 Hz, POMe), 58.10 (t, ³J_{CP}=7.7 Hz, C₁⁴CH₂CH₂), 115.13 (q, ¹J_{CF}=286.3 Hz, COOCF₃), 120.81 (s, C₀²), 121.24 (d, ³J_{CP}=4.5 Hz, C₁²), 129.74 (s, C₀³), 130.10 (s, C₁³), 134.07 (s, C₀⁴), 136.98 (s, C₁⁴), 148.98 (d, ²J_{CP}=7.9 Hz, C₁¹), 149.88 (brs, C₀¹), 157.99 (q, ²J_{CF}=40.1 Hz, COOCF₃).

### Characterization of Compound 11b-G₁(NH₂)

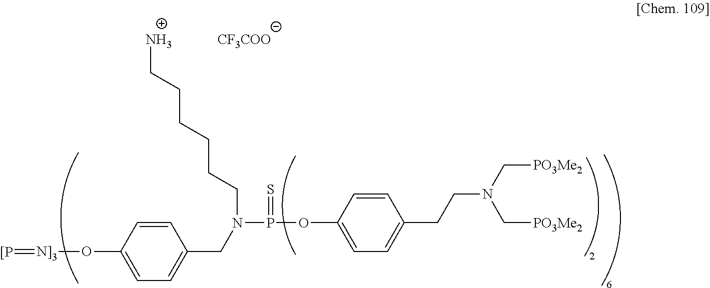

³¹P-{¹H} NMR (162 MHz, CD₃CN) δ (ppm): 8.71, 27.42, 68.21).

### Characterization of Compound 11c-G₁(NH₂)

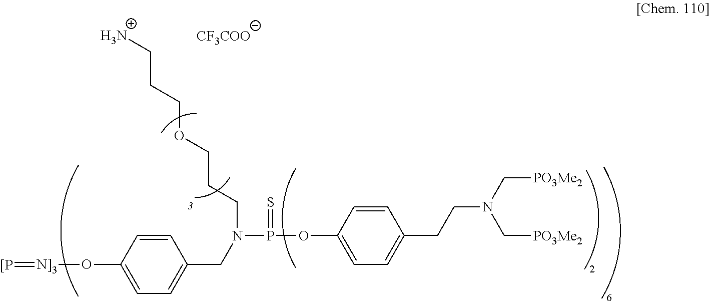

¹H NMR (400 MHz, CD₃CN) δ (ppm): 1.73 (m, 12H, PNCH₂CH₂), 1.76-1.86 (m, 12H, CH₂CH₂NHBoc), 2.87 (m, 24H, C₁⁴CH₂), 3.03 (m, 12H, N₀CHH₂H₂), 3.13-3.22 (m, 24H, C₁⁴CH₂CH₂), 3.31 (m, 24H, CH₂O), 3.38 (m, 12H, CH₂CH₂NHBoc), 3.42-3.65 (m, CH₂POMe, OCH₂CH₂O,), 3.79 (d, ²J_{HP}=10.9 Hz, 144H, POMe), 4.59 (d, ³J_{HP}=12.8 Hz, 12H, C₀⁴CH₂), 6.92 (d, ³J_{HH}=8.3 Hz, 12H, C₀²H), 7.14 (d, ³J_{HH}=7.8 Hz, 24H, C₁²H), 7.24 (d, ³J_{HH}=8.4 Hz, 24H, C₁³H), 7.27-7.36 (d, ³J_{HH}=8.3 Hz, 12H, C₀³H).

³¹P-{¹H} NMR (162 MHz, CD₃CN) δ (ppm): 8.89 (s, P₀), 24.44 (s, POMe), 68.26 (s, P₁).

### Synthesis of Compounds 11a-G₁(NH-NAC), 11b-G₁(NH-NAC) and 11c-G₁(NHNAC)

DIPEA is added to a solution of dendrimer 11-G1(NH2) (0.05 mmol) in a DCM/THF mixture until a neutral pH is obtained. Next, a solution of (N-acetyl-S-((3-((2,5-dioxopyrrolidin-1-yl)oxy)-3-oxopropyl)thio)cysteine (0.35 mmol) in THF is added. After 3 hours of stirring at ambient temperature, the mixture is concentrated to dryness. The crude residue is subsequently washed 3 times with 15 mL of EtOAc and 2 times with an EtOAc/THF 70/30 mixture to obtain dendrimer 11-G1(NH-NAC). The yield is between 75% and 95%. (N-acetyl-S-((3-((2,5-dioxopyrrolidin-1-yl)oxy)-3-oxopropyl)thio)cysteine is obtained according to a procedure described in Cleland J. L. et al., Bioeng. Transl. Med. 2018, 3, 87-101.

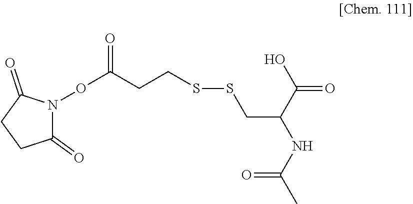 [SMILES: CC(=O)NC(CSSCCC(=O)ON1C(=O)CCC1=O)C(=O)O]

¹H NMR (400 MHz, THF) δ (ppm): 1.93 (s, 3H, CH₃), 2.79 (s, 4H, C(O)CH₂CH₂C(O)), 2.99-3.33 (m, 6H, SCH₂CH*, C(O)CH₂CH₂S), 4.77 (ddd, ³J_{HH}=8.0, 7.3, 4.8 Hz, 1H, CH*), 7.52 (d, ³J_{HH}=7.9 Hz, 1H, NHC(O)CH₃).

### Characterization of Compound 11a-G₁(NH-NAC)

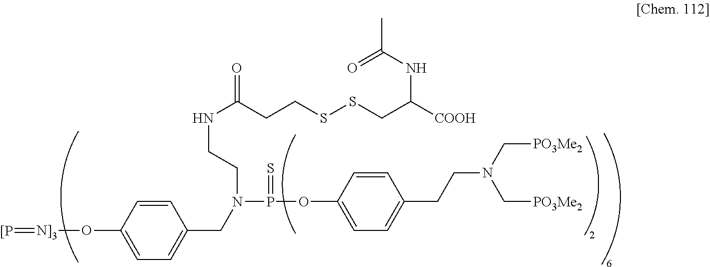

¹H NMR (600 MHz, CDCl₃) δ (ppm): 2.01 (s, 18H, NHCOCH₃), 2.37-2.42 (m, 12H, —SCH₂CH₂CONH), 2.72-2.94 (m, 36H, —SCH₂CH₂CONH, C₁⁴CH₂), 2.96-3.42 (m, 108H, C₁⁴CH₂ CH₂, NCH₂CH₂NHCO, —SCH₂H*, CH₂POMe), 3.75 (d, ²J_{HP}=10.5 Hz, 144H, POMe), 4.61 (d, ³J_{HP}=12.7 Hz, 12H, C₀⁴CH₂), 4.80 (m, 6H, CH*), 6.82-7.01 (m, 12H, C₀²H), 7.09 (d, ³J_{HH}=8.0 Hz, 24H, C₁²H), 7.20 (d, ³J_{HH}=7.9 Hz, 24H, C₁³H), 7.29 (d, ³J_{HH}=8.4 Hz, 12H, C₀³H).

³¹P-{¹H} NMR (243 MHz, CDCl₃) δ (ppm): 8.46 (s, P₀), 26.84 (s, POMe), 68.42 (s, P₁).

¹³C-{¹H} NMR (151 MHz, CDCl₃) δ (ppm): 23.01 (s, NHCOCH₃), 32.98 (s, C₁⁴CH₂), 33.97 (s, SCH2CH2NHCO), 35.81 (s, SCH₂CH₂NHCO), 37.41 (s, NCH₂CH₂NHCO), 41.52 (s, SCH₂CH*), 44.78 (NCH₂CH₂NHCO), 49.48 (dd, ¹J_{CP}=157.2, ³J_{CP}=7.1 Hz, CH₂POMe), 49.62 (brs, C₀⁴CH₂), 52.14 (s, SCH₂CH*), 52.86 (d, ²J_{CP}=7.2 Hz, POMe), 58.22 (t, ³J_{CP}=7.7 Hz, C₁⁴CH₂CH₂), 121.12 (s, C₀², C₁²), 129.84 (s, C₀³), 129.98 (s, C₁³), 133.84 (s, C₀⁴), 136.42 (s, C₁⁴), 149.29 (d, ²J_{CP}=7.9 Hz, C₁¹), 150.09 (brs, C₀¹), 170.64 (brs, NHCOCH₂CH₂S), 171.67 (brs, NHCOCH₃), 171.87 (brs, COOH).

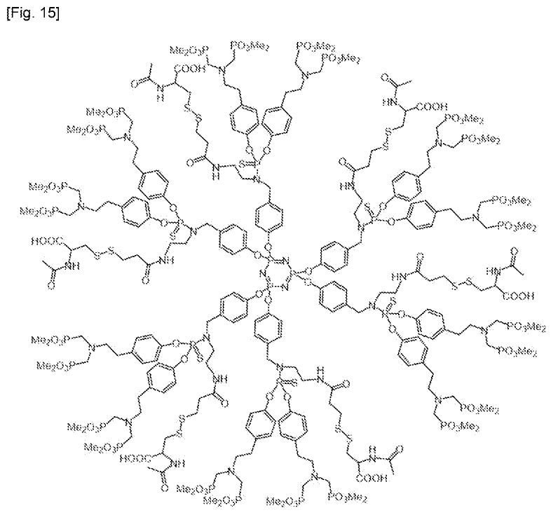

The structure of dendrimer 11a-G₁(NH-NAC) is represented in [FIG. 15].

### Characterization of Compound 11b-G₁ (NH-NAC)

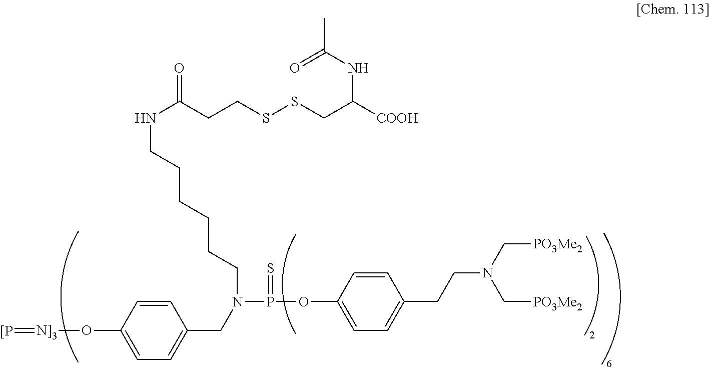

³¹P-{¹H} NMR (162 MHz, CDCl₃) δ (ppm): 8.41 (s, P₀), 26.61 (s, POMe), 68.21 (s, P₁).

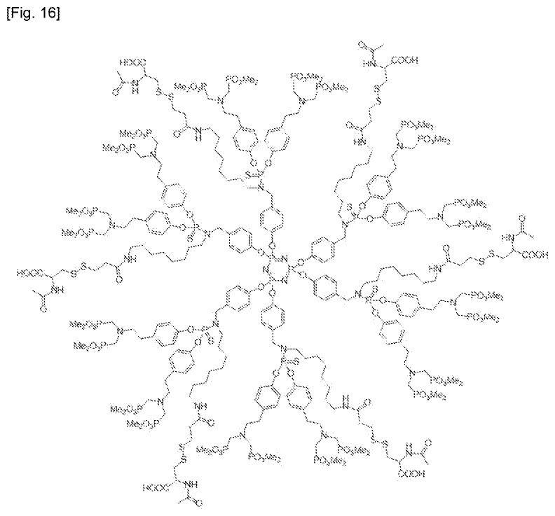

The structure of dendrimer 11 b-G₁(NH-NAC) is represented in [FIG. 16].

### Characterization of Compound 11c-G₁(NH-NAC)

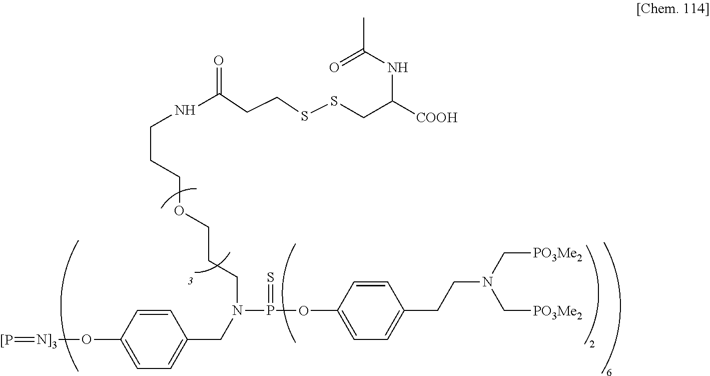

¹H NMR (400 MHz, CDCl₃) δ (ppm): 1.65-1.80 (m, 24H, CH₂CHH₂CH₂), 2.00 (s, 18H, NHCOCH₃), 2.53 (m, 12H, —SCH₂CH₂CONH), 2.69-2.79 (m, 24H, C₁⁴CH₂), 2.86-2.96 (m, 12H, —SCH₂CH₂CONH), 2.96-3.08 (m, 30H, C₁⁴CH₂ CH₂, —SCH₂CH*), 3.09-3.57 (m, N₀CH₂, C═ONHCH₂, —SCH₂H*, CH₂POMe, CH₂O), 3.72 (d, ²J_{HP}=10.5 Hz, 144H, POMe), 4.54 (d, ³J_{HP}=13.3 Hz, 12H, C₀⁴CH₂), 4.66 (m, 6H, CH*), 6.94-7.26 (m, 84H, NHCO, NHHOCH₃, C₀²H, C₁²H, C₀³H, C₁³H).

³¹P-{¹H} NMR (162 MHz, CDCl₃) δ (ppm): 8.13 (s, P₀), 26.84 (s, POMe), 68.06 (s, P₁).

¹³C-{¹H} NMR (101 MHz, CDCl₃) δ (ppm): 23.12 (s, 18H, NHCOCH₃), 27.99 (C═OHNCH₂CH₂), 29.05 (s, N₀CH₂CH₂), 32.96 (s, C₁⁴CH₂), 34.45 (s, SCH₂CH₂NHCO), 35.89 (s, SCH₂CH₂NHCO), 37.44 (s, NCH₂CH₂NHCO), 42.26 (s, SCH₂CH*), 43.12 (CH₂CH₂NHCO), 49.42 (dd, ¹J_{CP}=157.7, ³J_{CP}=7.3 Hz, CH₂POMe), 49.66 (brs, C₀⁴CH₂), 52.54-52.89 (m, POMe), 52.94 (brs, SCH₂CH*), 58.25 (t, ³J_{CP}=7.5 Hz, C₁⁴CH₂CH₂), 68.45 (s, CH₂O), 69.44 (s, CH₂O), 70.03 (s, CH₂O), 70.36 (s, CH₂O), 70.44 (s, CH₂O), 120.87 (s, C₀²), 121.04 (d, ³J_{CP}=4.6 Hz, C₁²), 129.47 (s, C₀³), 129.82 (s, C₁³), 134.28 (s, C₀⁴), 136.17 (s, C₁⁴), 149.46 (d, ²J_{CP}=7.9 Hz, C₁¹), 149.99 (s, C₀¹), 170.36 (brs, NHCOCH₂CH₂S), 171.23 (brs, NHCOCH₃), 172.75 (brs, COOH).

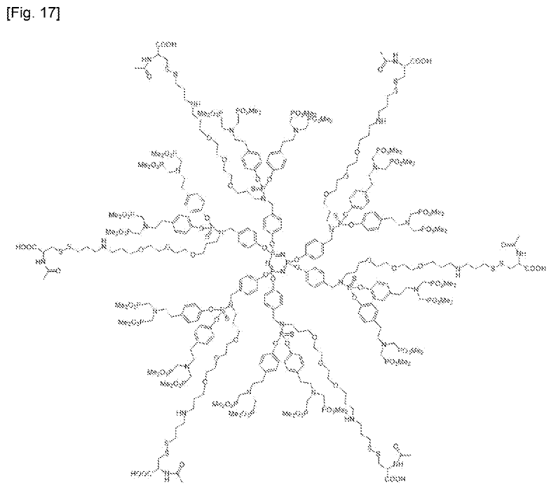

The structure of dendrimer 11c-G₁(NH-NAC) is represented in [FIG. 17].

### Synthesis of Compounds 11a-G₁ (NH-NAC/PO₃HNa), 11b-G₁ (NH-NAC/PO₃HNa) and 11c-G₁(NH-NAC/PO₃HNa)

Bromotrimethylsilane (0.7 mmol) is added dropwise to a solution of dendrimer 11-G1(NH₂-NAC) (0.01 mmol) in acetonitrile (10 mL), at a temperature of 0° C. After stirring overnight at ambient temperature, the solution is concentrated to dryness. Methanol (4 mL) is subsequently added to the dry residue. After one hour of stirring, the powder obtained is filtered then washed 2 times with 5 mL of methanol. The dendrimer is subsequently converted into its sodium salt by adding 1 equivalent of NaOH per surface phosphonic acid. The yield is greater than 95%.

### Characterization of Compound 11a-G₁(NH-NAC/PO₃HNa)

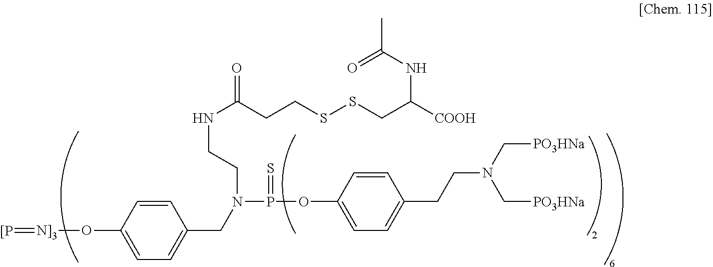

³¹P-{¹H} NMR (243 MHz, D₂O/CD₃CN) δ (ppm): 7.23 (s, P₀), 9.59 (s, POMe), 68.64 (s, P₁).

1H NMR (600 MHz, D₂O/CD₃CN) δ (ppm): 1.96 (s, 18H, NHCOCH₃), 2.32 (brs, 12H, 12H, —SCH₂CH₂CONH), 2.73 (brs, 12H, SCH₂CH₂CONH), 2.87 (m, 6H, CH₂CH*), 2.95-3.33 (m, 1H, C₁⁴CH₂CH₂, NCH₂CH₂NHCO, —SCH₂H*), 3.50-3.73 (m, 54H, C₁⁴CH₂CH₂, CH₂POH), 4.41-4.61 (m, 18H, C₀⁴CH₂, NHCHCOOH), 6.85 (s, 12H, C₀²H), 7.05 (m, 24H, C₁²H), 7.26 (brs, 36H, C₁³H, C₀³H).

¹³C-{¹H} NMR (151 MHz, D₂O/CD₃CN) δ (ppm): 22.05 (s, 18H, NHCOCH₃), 28.86 (s, C₁⁴CH₂), 33.16 (s, —SCH₂CH₂CONH), 35.13 (s, SCH₂CH₂NHCO), 37.38 (s, NCH₂CH₂NHCO), 39.62 (s, SCH₂CH*), 44.90 (NCH₂CH₂NHCO), 49.67 (brs, C₀⁴CH₂), 52.09 (s, CH₂POH), 53.02 (s, SCH₂CH*), 57.35 (brs, C₁⁴CH₂CH₂), 121.18 (s, C₀²), 121.62 (s, C₁²), 129.92 (s, C₀³), 130.48 (s, C₁³), 133.55 (s, C₁⁴), 134.42 (s, C₀⁴), 149.39 (m, C₀¹), 149.71 (m, C₁¹), 173.20 (brs, NCH₂CH₂NHCO), 173.48 (s, NHCOCH₃), 175.00 (brs, COOH).

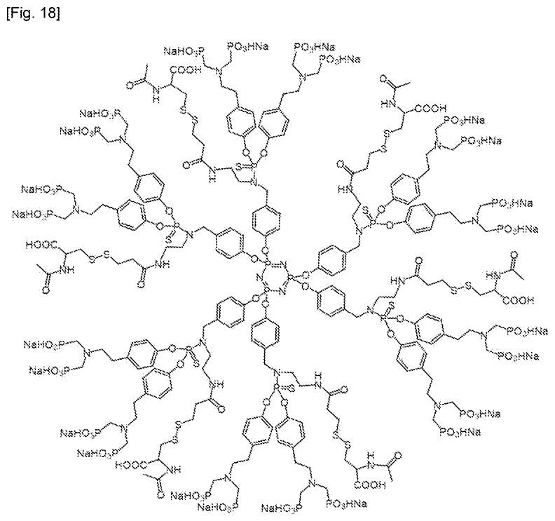

The structure of dendrimer 11a-G₁(NH-NAC/PO₃HNa) is represented in [FIG. 18].

### Characterization of Compound 11b-G₁(NH-NAC/PO₃HNa)

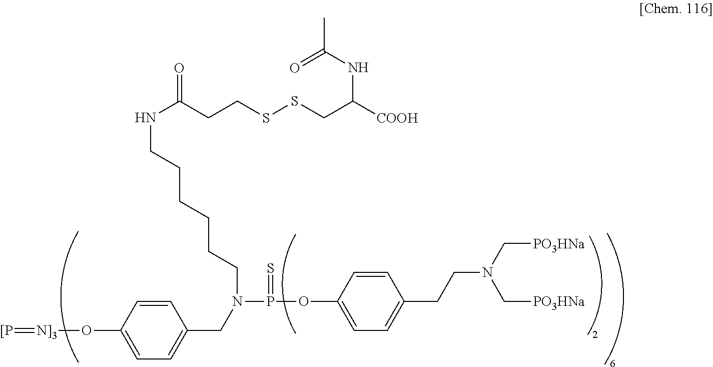

³¹P-{¹H} NMR (243 MHz, D₂O/CD₃CN) δ (ppm): 7.17 (s, P₀), 9.51 (s, POHNa), 68.69 (s, P₁).

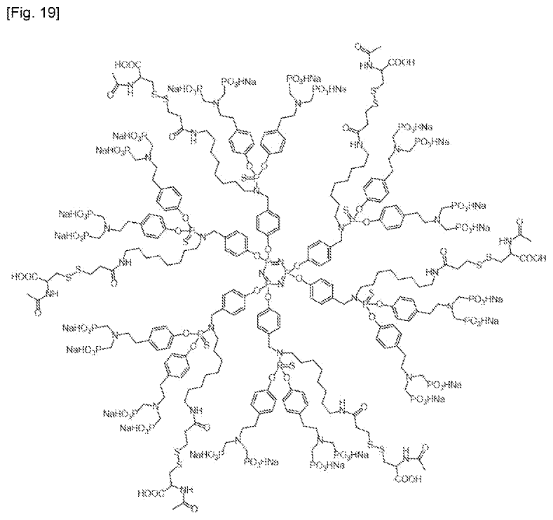

The structure of dendrimer 11b-G₁(NH-NAC/PO₃HNa) is represented in [FIG. 19].

Characterization of compound 11c-G₁(NH-NAC/PO₃HNa):

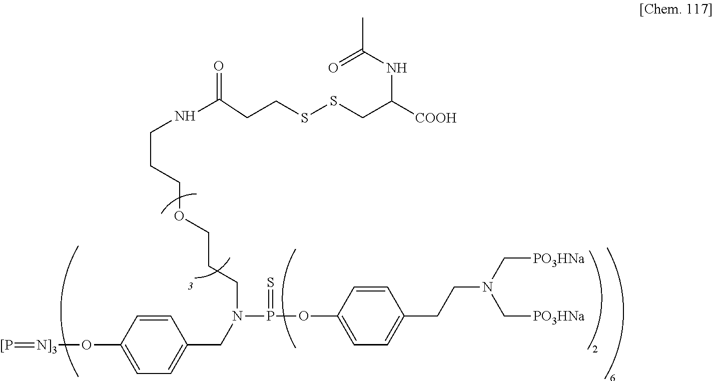

¹H NMR (400 MHz, D₂O) δ (ppm): 1.63-1.80 (m, 24H, CH₂CH₂CH₂), 2.43-2.66 (m, 12H, SCH₂CH₂CONH), 2.75-3.82 (m, 192H, CH₂), 4.48-4.62 (m, 36H, C₀⁴CH₂, CH*), 6.78-7.48 (m, 72H, CH-arom).

³¹P-{¹H} NMR (162 MHz, D₂O) δ (ppm): 7.18 (s, POHNa), 9.63 (s, P₀), 68.33 (s, P₁).

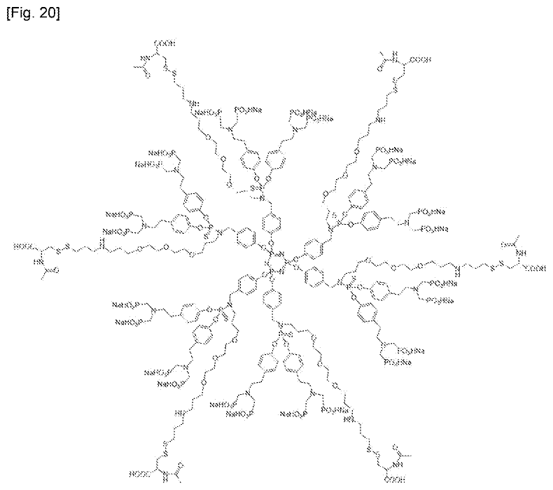

The structure of dendrimer 11c-G₁(NH-NAC/PO₃HNa) is represented in [FIG. 20].

### Synthesis of Layer-Block Dendrimers

Scheme 6 describes the reaction scheme of a layer-block dendrimer in which D′ is methyl and D″ is (CH₂—CH₂—O)₃—CH₃:

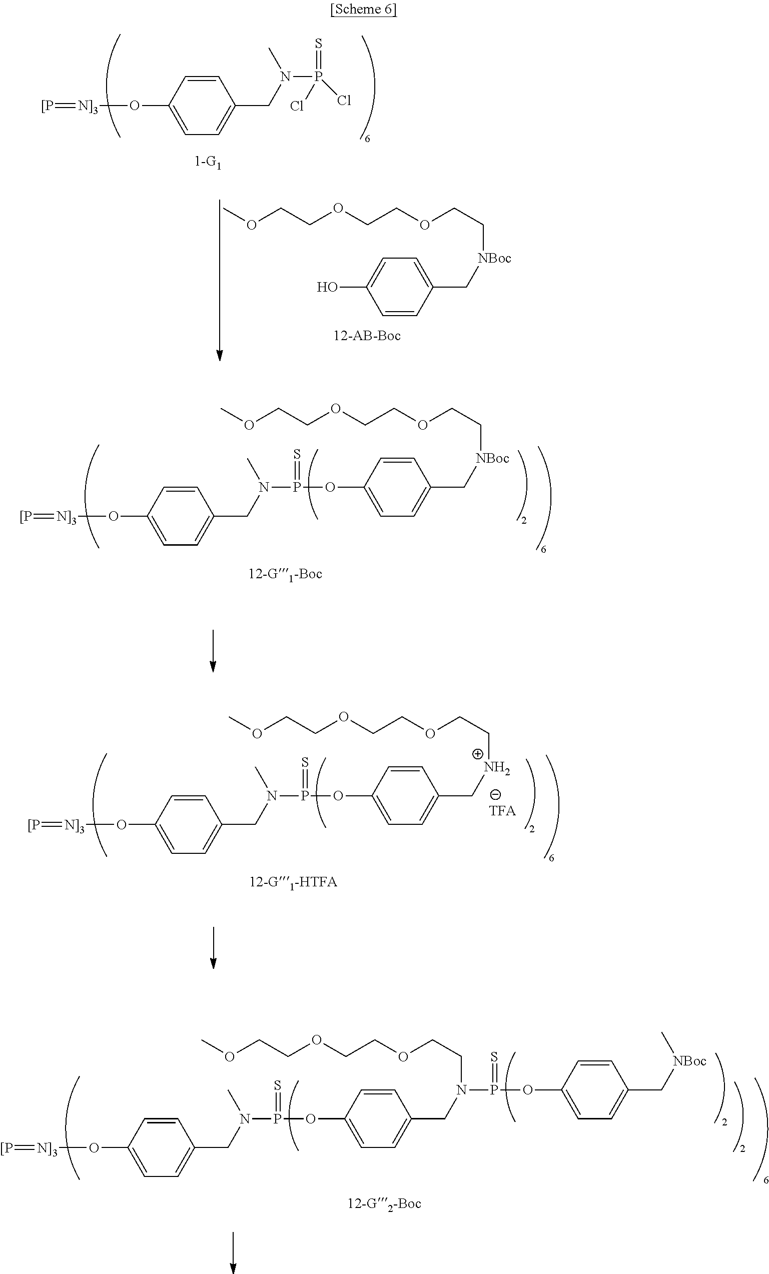

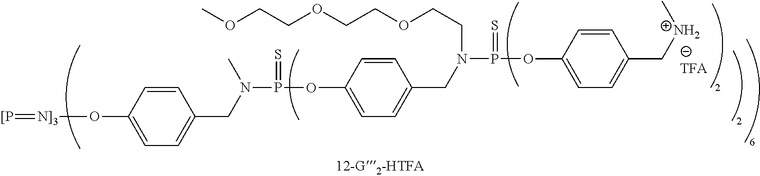

### Synthesis of Compound 12-G′″₁-Boc from 1-G₁

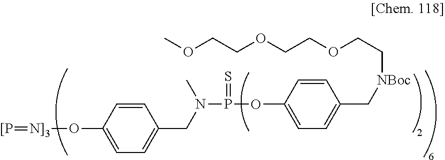

Cs₂CO₃ (693 mg, 2.130 mmol) and 12-AB-Boc (394 mg, 1.065 mmol) are added in succession to a solution of dendrimer 1-G₁ (125 mg, 0.071 mmol) in THF (20 mL). After stirring 48 hrs at ambient temperature, the mixture is centrifuged, filtered then concentrated under reduced pressure. The product is subsequently purified using silica gel chromatography to give dendrimer 12-G′″₁-Boc in the form of a transparent oil at a yield of 56%.

¹³C-{¹H} NMR (101 MHz, CDCl₃) δ (ppm): 28.44, 33.65, 46.19, 50.23, 51.06, 59.02, 69.72, 70.62, 71.94, 79.83, 120.99, 128.35, 128.91, 129.32, 134.08, 150.07, 155.78.

¹H NMR (400 MHz, CDCl₃) δ (ppm): 1.46-1.48 (m, 108H), 2.78 (d, J=11.0 Hz, 18H), 3.20-3.47 (m, 60H), 3.47-3.89 (m, 120H), 4.50 (s, 36H), 6.98 (d, J=8.6 Hz, 12H), 7.06-7.26 (m, 60H).

³¹P-{¹H} NMR (162 MHz, CDCl₃) δ (ppm): 8.28, 68.11.

### Synthesis of Compound 12-G′″₁-HTFA from 12-G′″₁-Boc

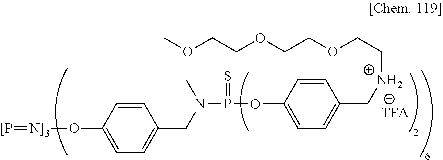

Compound 12-G′″₁-Boc (120 mg, 0.021 mmol) is dissolved in a CH₂Cl₂/trifluoroacetic acid mixture with a ratio between 50/50 and 70/30 (5 mL). After 30 min of stirring at ambient temperature, the mixture is concentrated to dryness under reduced pressure. The residue is subsequently co-evaporated 5 times with 3 mL of CH₂Cl₂ to give dendrimer 12-G′″₁-HTFA in the form of a yellow oil. The yield is greater than 90%.

¹H NMR (400 MHz, CDCl₃) δ (ppm): 2.70 (d, J=11.0 Hz, 18H), 3.15 (s, 24H), 3.30 (s, 36H), 3.56-3.67 (m, 72H), 3.77 (s, 24H), 4.10 (s, 24H), 4.37 (d, J=12.2 Hz, 12H), 6.88 (d, J=9.0 Hz, 12H), 7.15 (t, J=6.8 Hz, 36H), 7.42 (d, J=8.7 Hz, 24H).

¹³C-{¹H} NMR (101 MHz, CDCl₃) δ (ppm): 33.56, 46.19, 50.57, 58.73, 70.09 (d, J=8.5 Hz), 71.52, 114.49 (q), 121.53, 127.62, 129.17, 131.63, 133.73, 149.87, 151.74 (d, J=7.4 Hz), 160.68.

³¹P-{¹H} NMR (162 MHz, CDCl₃) δ (ppm): 8.94, 66.97.

### Synthesis of Compound 12-G′″₂-Boc from 12-G′″₁-HTFA with synthon 1-AB₂-Boc

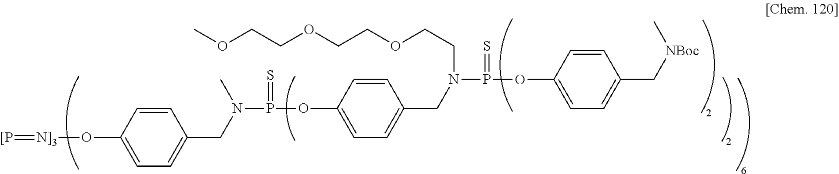

To a 120-mg solution of the trifluoroacetic acid salt of dendrimer 12-G′″₁-HTFA (0.021 mmol) in CH₂Cl₂ (20 to 50 mL) with DIPEA (0.135 mL, 0.756 mmol), compound 1-AB₂-Boc (180 mg, 0.315 mmol) is added. After 72 hours of stirring at 35° C., the mixture is concentrated to dryness under reduced pressure at ambient temperature. The crude residue is subsequently dissolved in ethyl acetate, filtered, then precipitated in an ether/pentane mixture to give compound 12-G′″₂-Boc in the form of a yellow oil at a yield of 80%.

³¹P-{¹H} NMR (121 MHz, CDCl₃) δ (ppm): 8.42, 67.78, 68.00.

### Synthesis of Compound 12-G′″₂-HTFA from 12-G′″₂-Boc

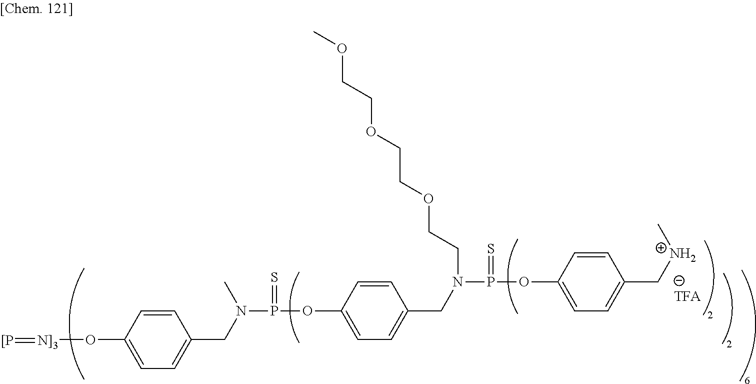

Compound 12-G′″₂-Boc (100 mg) is dissolved in a CH₂Cl₂/trifluoroacetic acid mixture with a ratio between 50/50 and 70/30 (5 mL). After 30 min of stirring at ambient temperature, the mixture is concentrated to dryness under reduced pressure. The residue is subsequently co-evaporated 5 times with 3 mL of CH₂Cl₂ to give dendrimer 12-G′″₂-HTFA in the form of a yellow oil. The yield is greater than 90%.

³¹P-{¹H} NMR (162 MHz, CDCl₃) δ (ppm): 8.91, 66.81, 67.1).

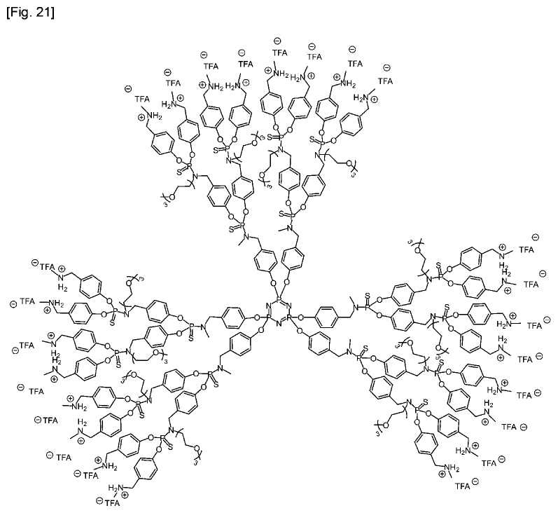

The structure of dendrimer 12-G′″₂-HTFA is represented in [FIG. 21].

Scheme 7 describes the reaction scheme of a layer-block dendrimer wherein D′ is methyl and D″ is octyl:

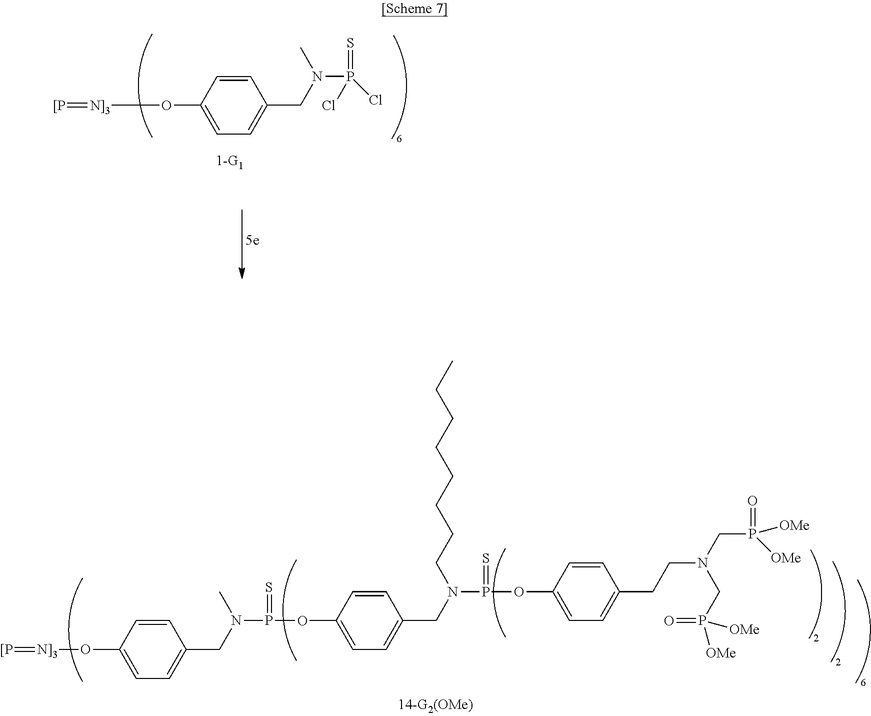

### Synthesis of Compound 13-G₂(OMe) from 1-G₁ with the synthon 5e

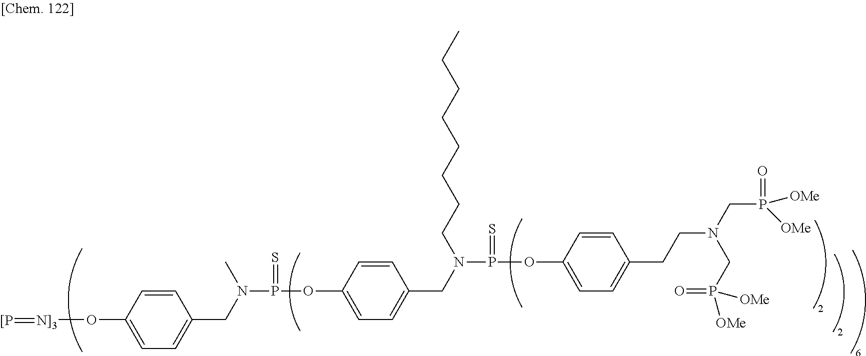

The compound 1-G₁ (41.2 mg, 0.0235 mmol) is added to a solution of compound 5e (300 mg, 0.282 mmol) in THF (15 mL) with Cs₂CO₃ (184 mg, 0.564 mmol). After stirring overnight at 40° C., the mixture is centrifuged, filtered then concentrated under reduced pressure. Dendrimer 13-G₂(OMe) was obtained in the form of a transparent oil at a yield of 90%.

¹H NMR (400 MHz, CDCl₃) δ (ppm): 0.69-0.96 (m, 36H), 1.08-1.40 (m, 120H), 2.73-2.85 (m, 72H), 3.06 (t, J=7.8 Hz, 48H), 3.20 (d, J=9.3 Hz, 114H), 3.74 (d, J=10.3 Hz, 288H), 4.37-4.74 (m, 36H), 6.89-7.39 (m, 168H).

¹³C-{¹H} NMR (101 MHz, CDCl₃) δ (ppm): 14.10 (d, J=2.6 Hz), 22.59, 26.36-27.03 (m), 27.62, 29.19 (dd, J=14.2, 6.5 Hz), 31.74 (d, J=7.1 Hz), 33.14 (d, J=18.8 Hz), 44.59, 45.86, 48.29-49.03 (m), 49.51, 50.28 (t, J=7.5 Hz), 52.26-52.97 (m), 57.83-58.79 (m), 121.01 (d, J=4.8 Hz), 121.39 (d, J=4.7 Hz), 127.63-130.62 (m), 135.73 (d, J=1.9 Hz), 136.09, 149.32 (d, J=7.2 Hz), 149.53 (d, J=7.3 Hz), 150.42 (d, J=7.7 Hz).

³¹P-{¹H} NMR (162 MHz, CDCl₃) δ (ppm): 7.97, 26.84, 68.07, 68.68.

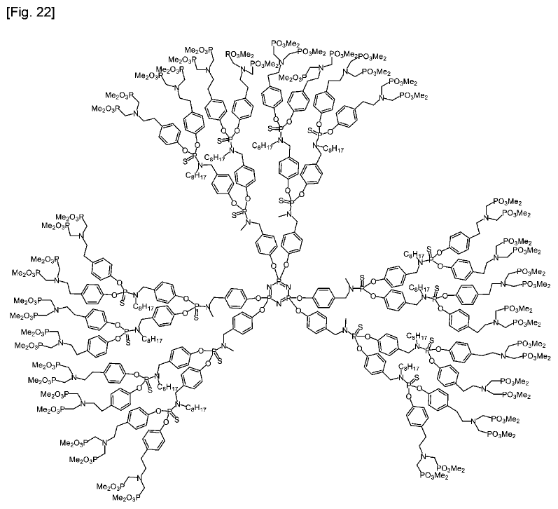

The structure of dendrimer 13-G₂(OMe) is represented in [FIG. 22].

### Evaluation of the Stability of the Dendrimers According to the Invention

The stability of the dendrimers according to the invention at physiological pH was evaluated in comparison with that of a phosphorous dendrimer of the polyphosphorhydrazone type from the prior art (WO 2005/052031 A1).

Procedure

The compounds were dissolved in water or in an H₂O/D₂O mixture (20 to 50 mg/mL). If necessary, the pH was adjusted and the solutions were stored at 25° C. away from light between each NMR spectrum recording.

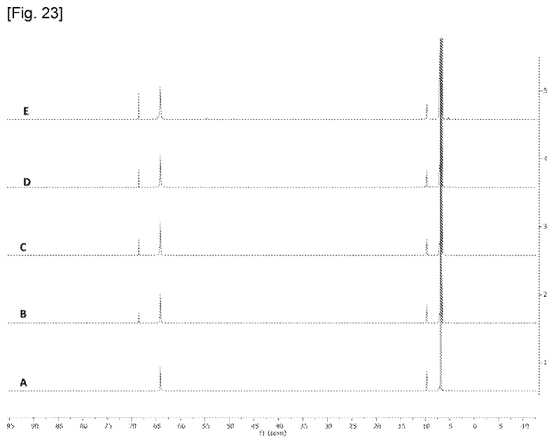

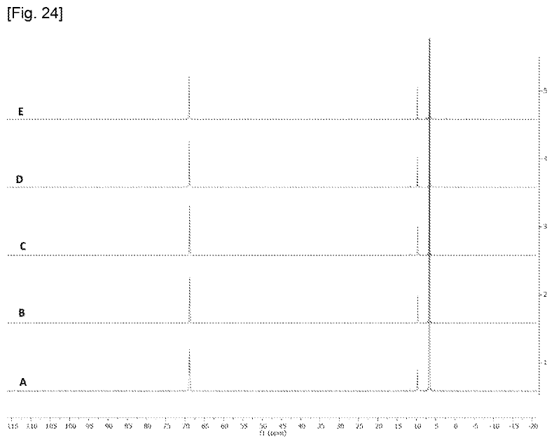

The results show that the stability of compound 3-G′1(ONa) according to the invention at pH=7.2 is far greater than that of the reference compound ABP having a polyphosphorhydrazone backbone and aminobismethylene phosphonic acid terminations (monosodium salt) derived from tyramine. ³¹p NMR spectroscopy shows a degradation of the compound ABP after 4 days at pH=7.2 at 25° C. (spectrum B of [FIG. 23], with the signal confirming hydrolysis at around 67 ppm; this signal intensifies over time, as indicated in spectra C, D and E). Under the same conditions, compound 3-G′1(ONa) remains perfectly stable and no by-product is observed even after 8 months of storage in solution (spectra A to E of [FIG. 24]).

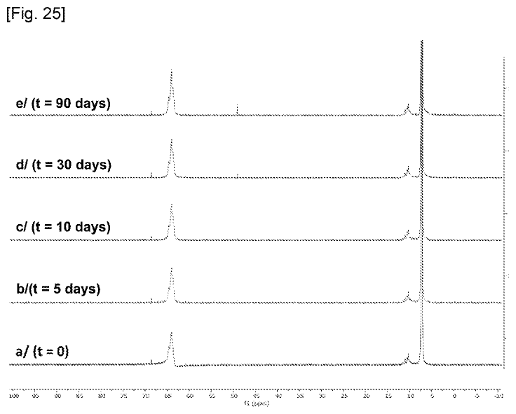

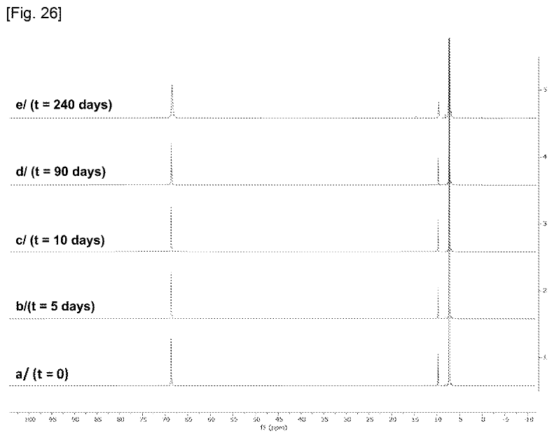

The stability of compound 3-G′1(ONa) according to the invention at pH=5 is far greater than that of the reference compound ABP having a polyphosphorhydrazone backbone and aminobismethylene phosphonic acid terminations (monosodium salt) derived from tyramine. ³¹P NMR spectroscopy shows a degradation of the compound ABP after 4 days at pH=5 at 25° C. (spectrum B of [FIG. 25], with the signal confirming hydrolysis at around 67 ppm; this signal intensifies overtime, as indicated in spectra C, D and E). From 10 days onwards, a second signal attesting to degradation is also observed at 48 ppm (spectrum C of [FIG. 25]; this signal intensifies with time as shown in spectra C, D and E). Under the same conditions, compound 3-G′1(ONa) remains perfectly stable and no by-product is observed even after 8 months of storage in solution (spectra A to E of [FIG. 26]).

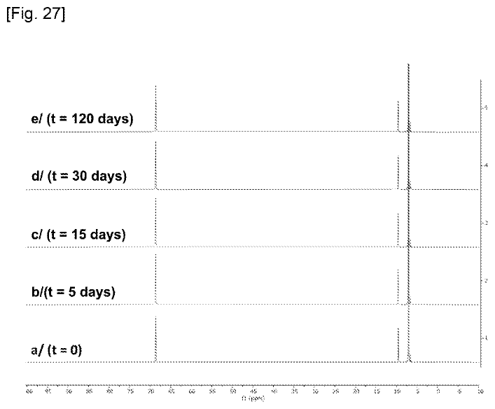

The stability of compound 4-G′1(ONa) according to the invention at pH=7 is far greater than that of the reference compound ABP having a polyphosphorhydrazone backbone and aminobismethylene phosphonic acid terminations (monosodium salt) derived from tyramine. ³¹P NMR spectroscopy shows a degradation of the compound ABP after 4 days at pH=7 at 25° C. (spectrum B of [FIG. 23], with the signal confirming hydrolysis at around 67 ppm; this signal intensifies over time, as indicated in spectra C, D and E). Under the same conditions, compound 4-G′1(ONa) remains perfectly stable and no by-product is observed even after 4 months of storage in solution (spectra A to E of [FIG. 27]).

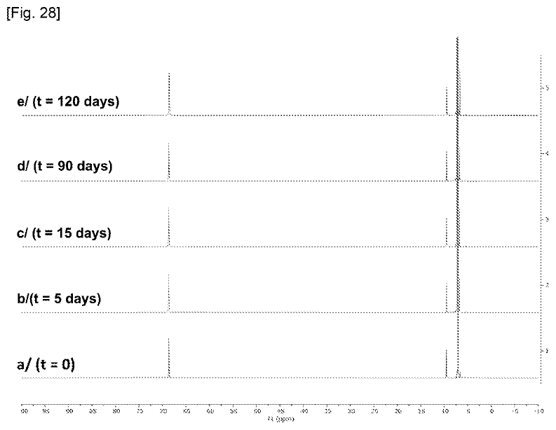

The stability of compound 5-G′1(ONa) according to the invention at pH=7 is far greater than that of the reference compound ABP having a polyphosphorhydrazone backbone and aminobismethylene phosphonic acid terminations (monosodium salt) derived from tyramine. ³¹P NMR spectroscopy shows a degradation of the compound ABP after 4 days at pH=7 at 25° C. (spectrum B of [FIG. 23], with the signal confirming hydrolysis at around 67 ppm; this signal intensifies over time, as indicated in spectra C, D and E). Under the same conditions, compound 5-G′1(ONa) remains perfectly stable and no by-product is observed even after 4 months of storage in solution (spectra A to E of [FIG. 28]).

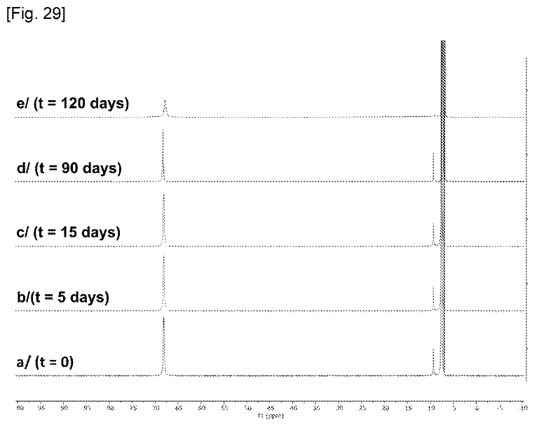

The stability of compound 5-G′1(ONa) according to the invention at pH=4 is far greater than that of the reference compound ABP having a polyphosphorhydrazone backbone and aminobismethylene phosphonic acid terminations (monosodium salt) derived from tyramine at pH=5. ³¹P NMR spectroscopy shows a degradation of the compound ABP after 4 days at pH=5 at 25° C. (spectrum B of [FIG. 25], with the signal confirming hydrolysis at around 67 ppm; this signal intensifies over time, as indicated in spectra C, D and E). At pH=4 and at 25° C., compound 5-G′1(ONa) remains perfectly stable and no by-product is observed even after 4 months of storage in solution (spectra A to E of [FIG. 29]).

### Biological Evaluation

The activation of primary human monocytes of dendrimer 3-G′1(ONa) according to the invention was evaluated.

Procedure

Human blood from healthy donors is recovered from the établissement français du sang [French Blood Service] (EFS, Toulouse, France). Peripheral blood mononuclear cells (PBMCs) are subsequently separated from the blood using a density gradient with a Pancoll solution (PANBiotech GmbH) by centrifugation at 1200 rpm for 20 min at 20° C. From the PBMCs, the monocytes are isolated by negative selected with antibodies directed against all blood cells (T cells, B cells, NK cells, dendritic cells, erythrocytes and granulocytes) except for monocytes, using the kit Dynabeads® Untouched™ Human Monocytes (Invitrogen). Monocyte purity was verified by flow cytometry to be greater than 90% for each donor with an anti-CD14-APC-Cy7 antibody (Miltenyi Biotec).

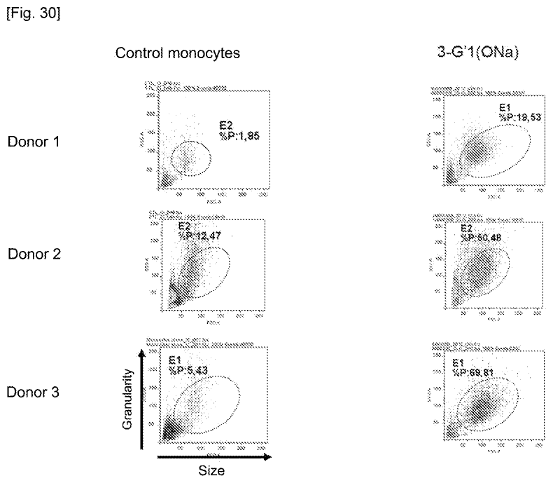

The freshly purified monocytes were resuspended in a 48-well plate at 1 million per mL in RPMI 1640+GLUTAMAX, penicillin and streptomycin at 100 U/mL and 10% fetal bovine serum (FBS). The dendrimers were added at the start of the cultures, at a concentration of 20 μM. After 5 days of culture at 37° C., the morphology of the monocytes was analyzed by flow cytometry with a MACSQUANT Q10 cytometer (Miltenyi Biotec). All the cytometry data were analyzed by the Flowlogic™ software (Miltenyi Biotec).

[FIG. 30] shows the results of the flow cytometry of the morphology (granularity and size) of the monocytes (on the graphs, each dot is a cell). 3 donors were tested, the dendrimer concentration is 20 μM. The left-hand column relates to the control, non-activated, monocytes. The right-hand column relates to the monocytes cultured for 4 days with dendrimer 3-G′1(ONa): the activated monocytes are within the ellipse.

A change in morphology (increase in granularity and size) reflects activation of the primary human monocytes. The results show that dendrimer 3-G′1(ONa) leads to an increase in granularity and in the size of the primary human monocytes. Moreover, it is noted that a higher proportion of monocytes is in the ellipse of the activated monocytes, with the proportion depending on the donor.

1. An n generation dendrimer or the pharmaceutically acceptable salts thereof, comprising:
a central core § of valence m, where m is an integer greater than or equal to 1;
branching generation chains around the core, represented by formula (CG): 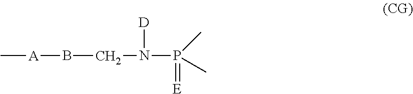
wherein
A is O or S;
B is aryl or heteroaryl, optionally substituted by C1-C6 alkyl or halo;
D is selected from C1-C12 alkyl, C1-C12 haloalkyl, aryl, heteroaryl, (CH₂—CH₂—O)ₐ—CH₃, CH₂—(CH₂—CH₂—O)ₐ—CH₃, (CH₂)_{b}—NR′R″ and CH₂—(CH₂—CH₂—O)_{c}—NR′R″, where a, b and c are, independently of one another, integers of between 1 and 12, and R′ and R″ are, independently of one another, selected from H, C1-C12 alkyl, C1-C12 haloalkyl, tert-butyloxycarbonyl, fluorenylmethoxycarbonyl, methoxycarbonyl, tert-butylcarbonyl, p-toluenesulfonyl, methylsulfonyl, trifluoromethylsulfonyl, allyl, benzyl, trityl, 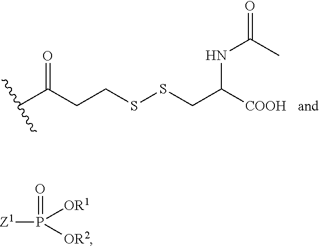
where Z¹ is a single bond or a linear or branched hydrocarbon chain having 1 to 6 chain members, one or more chain members optionally being a heteroatom, and R¹ and R² are, independently of one another, selected from H, C1-C6 alkyl and M⁺, where M⁺ is a cation;
E is O or S;
an intermediate chain at the end of each generation chain represented by the formula (CI):
-G-J-L- (CI)
wherein
G is O or S;
J is aryl or heteroaryl, optionally substituted by C1-C6 alkyl or halo;
L is a linear or branched hydrocarbon chain having 1 to 6 chain members, one or more chain members optionally being a heteroatom;
an end group at the end of each intermediate chain represented by the formula (T): 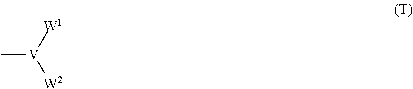
wherein
V is CH or N;
W¹ is H, C1-C12 alkyl or 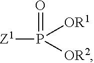
where Z¹ is a single bond or a linear or branched hydrocarbon chain having 1 to 6 chain members, one or more chain members optionally being a heteroatom, and R¹ and R² are, independently of one another, selected from H, C1-C6 alkyl and M⁺, where M⁺ is a cation;
W² is C1-C12 alkyl or 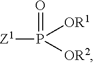
where Z¹ is a single bond or a linear or branched hydrocarbon chain having 1 to 6 chain members, one or more chain members optionally being a heteroatom, and R¹ and R² are, independently of one another, selected from H, C1-C6 alkyl and M⁺, where M⁺ is a cation;
wherein n is an integer of between 1 and 12.

2. The dendrimer according to claim 1, wherein the central core § is selected from pentoses, hexoses, and the groups of the following formulae:

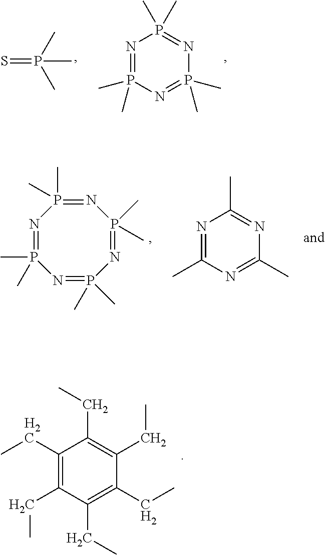

3. The dendrimer according to claim 1 wherein m is equal to 6.

4. The dendrimer according to claim 1 wherein that n is an integer between 1 and 5.

5. The dendrimer according to claim 1 wherein in formula (CG), A is O.

6. The dendrimer according to claim 1 wherein in formula (CG), B is phenyl, optionally substituted by C1-C6 alkyl or halo.

7. The dendrimer according to claim 1 wherein in formula (CG), E is S.

8. The dendrimer according to claim 1 wherein in formula (CI), G is O.

9. The dendrimer according to claim 1 wherein in formula (CI), J is phenyl, optionally substituted by C1-C6 alkyl or halo.

10. The dendrimer according to claim 1 wherein in formula (CI), L is (CH₂)_{d}, where d is an integer between 1 and 6.

11. The dendrimer according to claim 1 wherein in formula (T), V is N.

12. The dendrimer according to claim 1 wherein in formula (T), W¹ is H and W² is C1-C12 alkyl.

13. The dendrimer according to claim 1 wherein in formula (T), W¹ and W² are 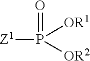
where Z¹ is a single bond or a linear or branched hydrocarbon chain having 1 to 6 chain members, and R¹ and R² are, independently of one another, selected from H, C1-C6 alkyl and M⁺, where M⁺ is a cation of an element from group IA, IIA, IIB or IIIA of the periodic table of the elements, or a cation of a nitrogenous base, preferably M⁺ is Na⁺ or K⁺.

14. The dendrimer according to claim 1 represented by the following formula (II): 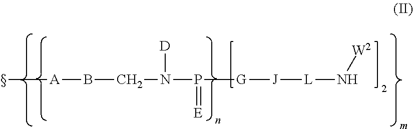
or the pharmaceutically acceptable salts thereof,
wherein
§, A, B, D, E, G, J, L, W², m and n are as defined in claim 1, and
{ }ₙ denotes the branching structure of the n generation chains of said dendrimer.

15. The dendrimer according to claim 1 represented by the following formula (III): 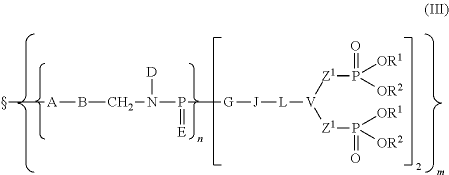
or the pharmaceutically acceptable salts thereof,
wherein
§, A, B, D, E, G, J, L, V, Z¹, R¹, R², m and n are as defined in claim 1, and
{ }ₙ denotes the branching structure of the n generation chains of said dendrimer.

16. The dendrimer according to claim 1 represented by the following formula (IV): 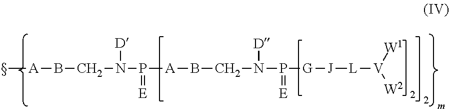
or the pharmaceutically acceptable salts thereof,
wherein
§, A, B, E, G, J, L, V, W¹, W², m are as defined previously or in formula (I), and
D′ and D″ are independently selected from C1-C12 alkyl, C1-C12 haloalkyl, aryl, heteroaryl, (CH₂—CH₂—O)ₐ—CH₃, CH₂—(CH₂—CH₂—O)ₐ—CH₃, (CH₂)_{b}—NR′R″ and CH₂—(CH₂—CH₂—O)_{c}—CH₂—CH₂—CH₂—NR′R″, where a, b and c are, independently of one another, integers of between 1 and 12, and R′ and R″ are, independently of one another, selected from H, C1-C12 alkyl, C1-C12 haloalkyl, tert-butyloxycarbonyl, fluorenylmethoxycarbonyl, methoxycarbonyl, tert-butylcarbonyl, p-toluenesulfonyl, methylsulfonyl, trifluoromethylsulfonyl, allyl, benzyl, trityl, 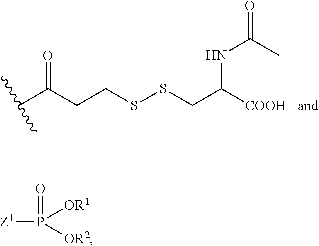
where Z¹ is a single bond or a linear or branched hydrocarbon chain having 1 to 6 chain members, one or more chain members optionally being a heteroatom, and R¹ and R² are, independently of one another, selected from H, C1-C6 alkyl and M⁺, where M⁺ is a cation;

17. The dendrimer according to claim 1, selected from:

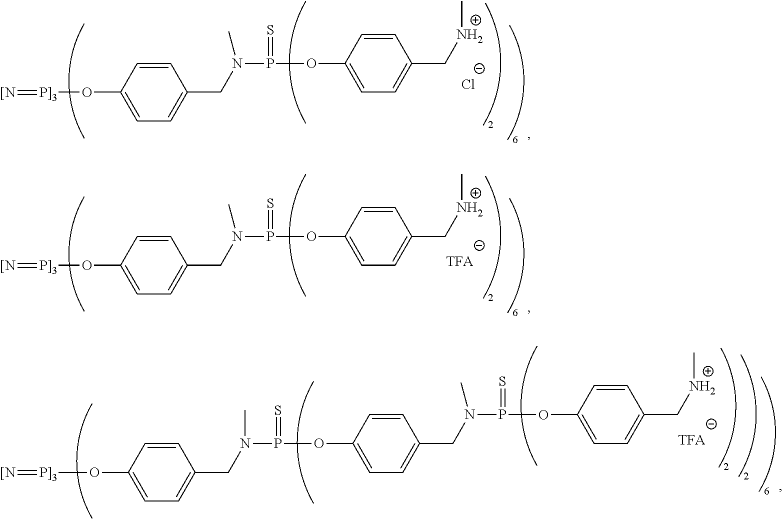

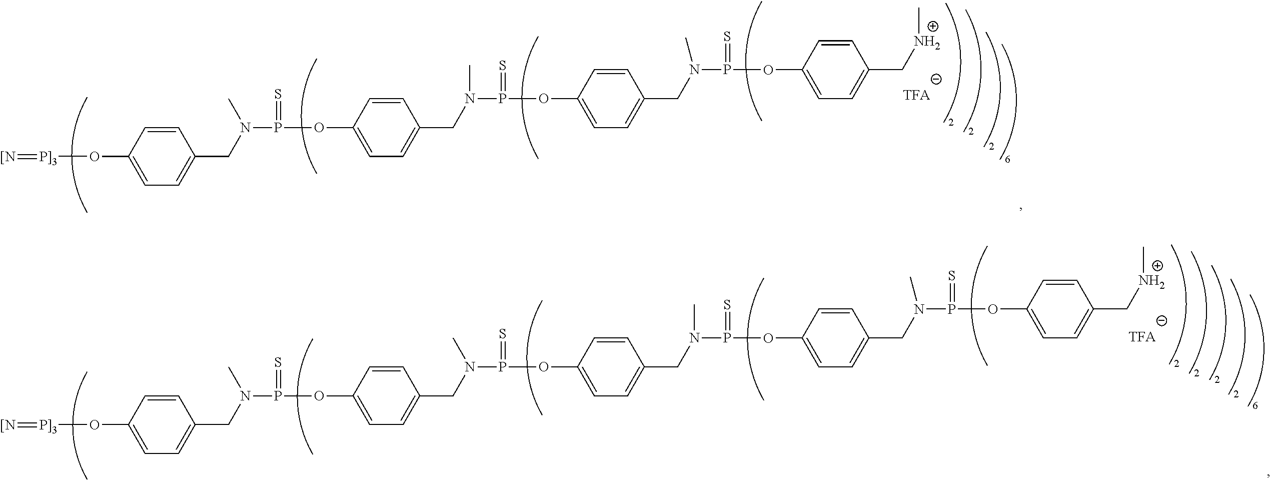

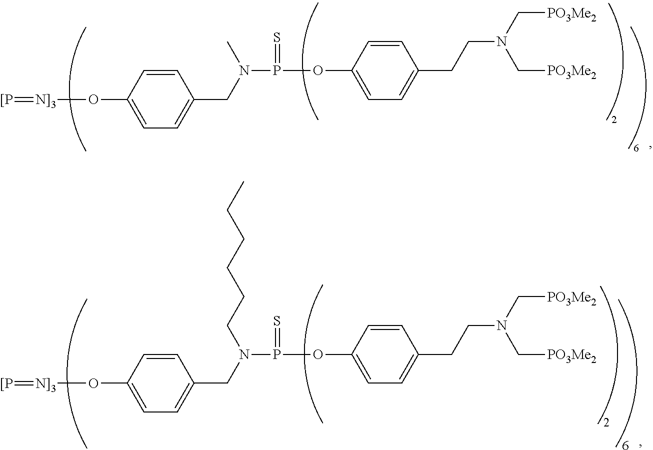

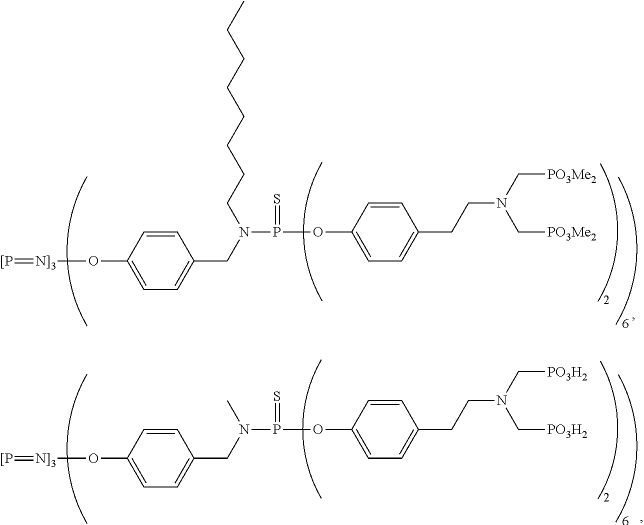

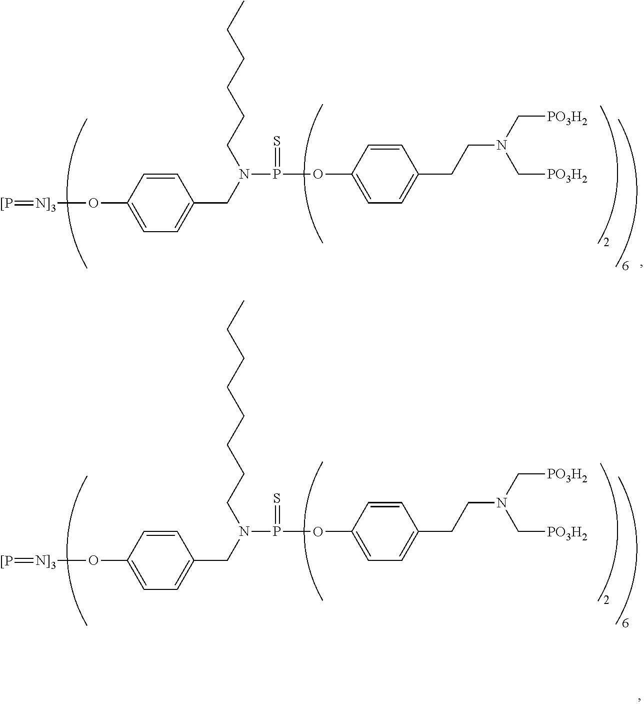

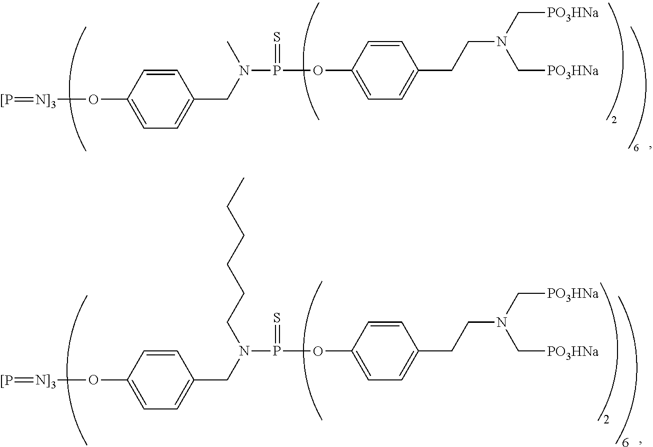

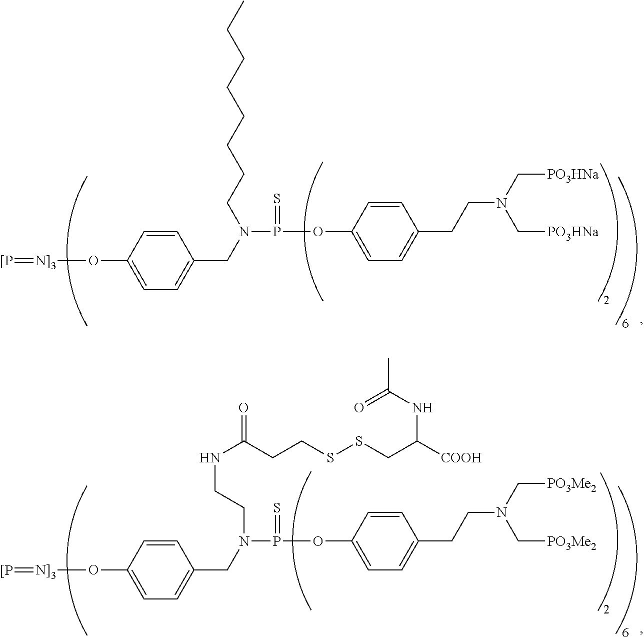

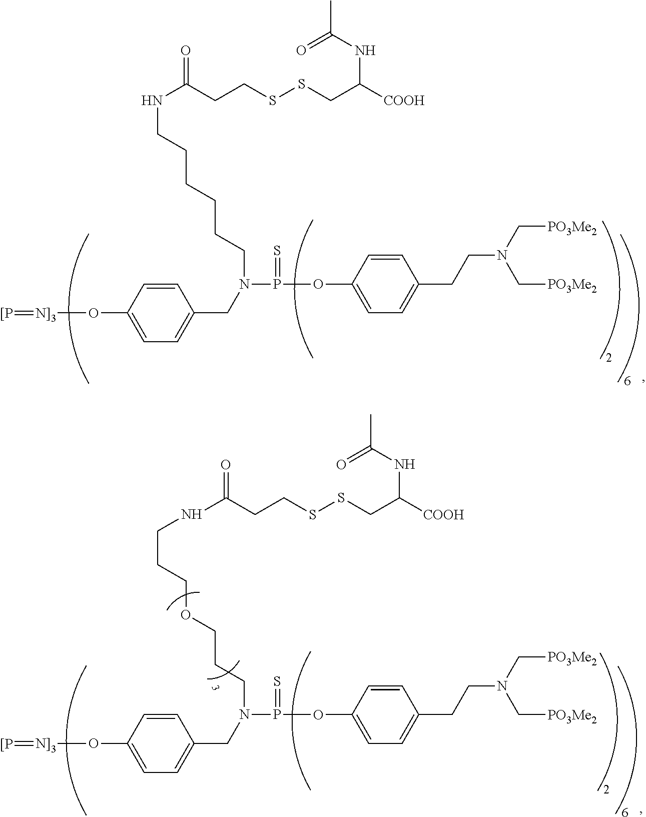

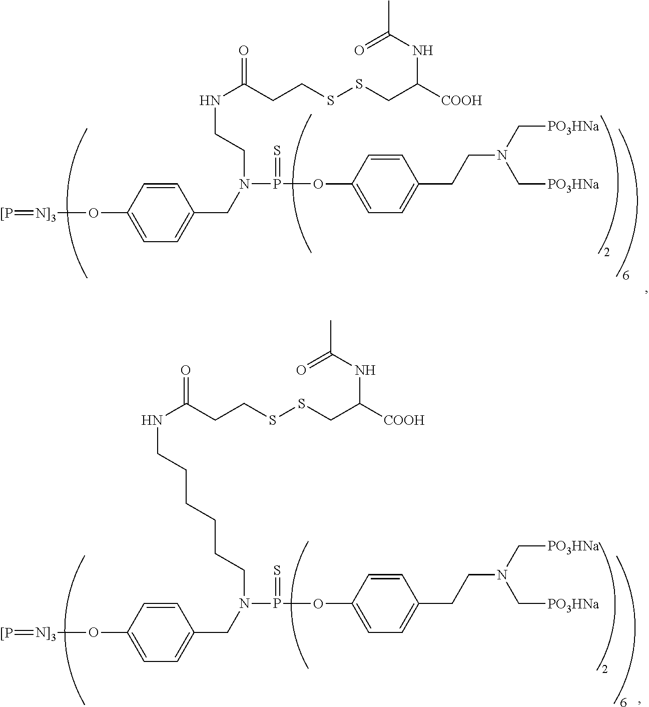

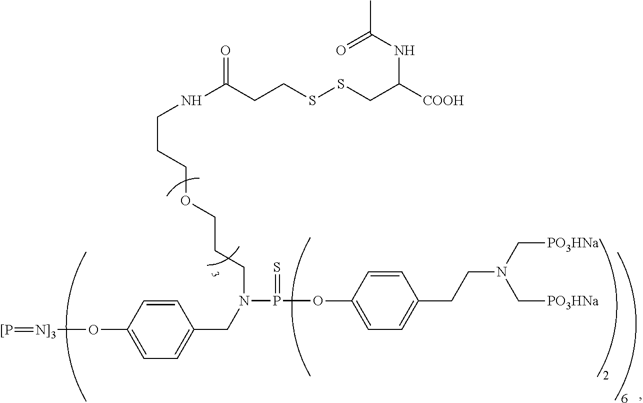

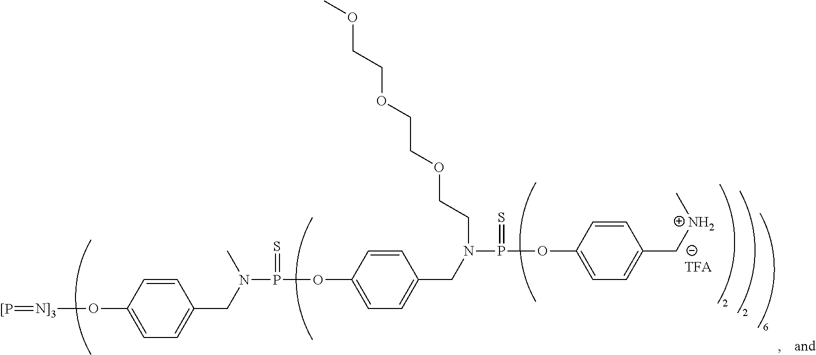

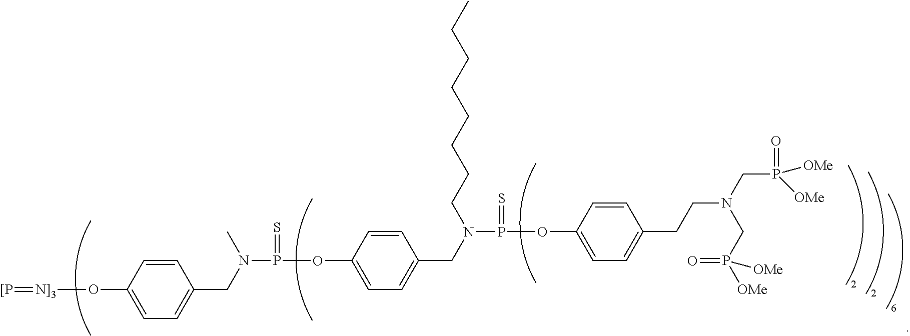

18. A method for preparing a dendrimer according to claim 1, comprising the following steps, in succession:
(a) preparing an intermediate of the following formula: 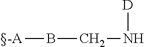
or a pharmaceutically acceptable salt thereof,
wherein
§, A, B and D are as defined in claim 1;
(b) reacting the intermediate obtained in step (a) with P(E)X₃, where E is O or S and X is a halogen;
(c) reacting the product obtained in step (b) with a fragment comprising an intermediate chain and an end group.

19. A method for preparing a dendrimer according to claim 1, comprising the following steps, in succession:
(a) preparing a fragment comprising a generation chain, an intermediate chain and an end group of the following formula: 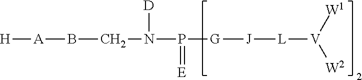
or a pharmaceutically acceptable salt thereof,
wherein
A, B, D, E, G, J, L, V, W¹ and W² are as defined in claim 1;
(b) reacting the fragment obtained in step (a) with a halogenated central core §.

20. A pharmaceutical composition comprising at least one dendrimer according to claim 1 and a pharmaceutically acceptable excipient.

21. A method of using the dendrimer according to claim 1 comprising providing the dendrimer to a subject as a medicament.

22. (canceled)

23. A method of using the pharmaceutical composition according to claim 20 comprising providing the pharmaceutical composition to a subject as a medicament.

24. A method of treating or preventing an inflammatory disease comprising administering to a subject a therapeutically effective amount of the dendrimer according to claim 1.

25. The method according to claim 24, wherein the inflammatory disease selected from chronic inflammatory diseases, autoimmune inflammatory diseases and pro-inflammatory and inflammatory conditions associated with a cancer.

26. A method of treating or preventing an inflammatory disease comprising administering to a subject a therapeutically effective amount of the pharmaceutical composition according to claim 20.

27. The method according to claim 26, wherein the inflammatory disease selected from chronic inflammatory diseases, autoimmune inflammatory diseases and pro-inflammatory and inflammatory conditions associated with a cancer.
